SEQUENCE LISTING (191 sequences)

SEQ ID NO: 1 - protein, 6 aa, synthetic construct
NIGSKS

SEQ ID NO: 2 - protein, 6 aa, synthetic construct
NIVTKS

SEQ ID NO: 3 - protein, 6 aa, synthetic construct
NIGVKS

SEQ ID NO: 4 - protein, 6 aa, synthetic construct
NIGMKS

SEQ ID NO: 5 - protein, 6 aa, synthetic construct
NIGLKS

SEQ ID NO: 6 - protein, 6 aa, synthetic construct
NIGSKM

SEQ ID NO: 7 - protein, 6 aa, synthetic construct
NIGVKA

SEQ ID NO: 8 - protein, 6 aa, synthetic construct
NIWGKS

SEQ ID NO: 9 - protein, 6 aa, synthetic construct
DIGKKS

SEQ ID NO: 10 - protein, 6 aa, synthetic construct
NIGYKS

SEQ ID NO: 11 - protein, 6 aa, synthetic construct
KLGDKW

SEQ ID NO: 12 - protein, 6 aa, synthetic construct
NIGFKS

SEQ ID NO: 13 - protein, 7 aa, synthetic construct
DDSDRPS

SEQ ID NO: 14 - protein, 7 aa, synthetic construct
DDSDRSP

SEQ ID NO: 15 - protein, 7 aa, synthetic construct
DDIDRPS

SEQ ID NO: 16 - protein, 7 aa, synthetic construct
DDIDRPA

SEQ ID NO: 17 - protein, 7 aa, synthetic construct
EDVDRPS

SEQ ID NO: 18 - protein, 9 aa, synthetic construct
QVWDSRTYV

SEQ ID NO: 19 - protein, 8 aa, synthetic construct
QVWDRGMI

SEQ ID NO: 20 - protein, 9 aa, synthetic construct
QVWGRYQMI

SEQ ID NO: 21 - protein, 11 aa, synthetic construct
QVWASTTAPSV

SEQ ID NO: 22 - protein, 9 aa, synthetic construct
QVWDSRSVV

SEQ ID NO: 23 - protein, 8 aa, synthetic construct
GFTFSSYS

SEQ ID NO: 24 - protein, 8 aa, synthetic construct
GFTFSNKA

SEQ ID NO: 25 - protein, 8 aa, synthetic construct
GFTFTSRA

SEQ ID NO: 26 - protein, 8 aa, synthetic construct
GFTFSSRS

SEQ ID NO: 27 - protein, 8 aa, synthetic construct
GFTYSSRA

SEQ ID NO: 28 - protein, 8 aa, synthetic construct
GFTFGDRA

SEQ ID NO: 29 - protein, 8 aa, synthetic construct
GFTFDNRA

SEQ ID NO: 30 - protein, 8 aa, synthetic construct
GFTFSDRA

SEQ ID NO: 31 - protein, 8 aa, synthetic construct
GFSFSSRA

SEQ ID NO: 32 - protein, 8 aa, synthetic construct
GLTFSSRA

SEQ ID NO: 33 - protein, 8 aa, synthetic construct
GFTFSTRA

SEQ ID NO: 34 - protein, 8 aa, synthetic construct
GFTFSSRA

SEQ ID NO: 35 - protein, 8 aa, synthetic construct
GFTFSRQA

SEQ ID NO: 36 - protein, 8 aa, synthetic construct
GLTFSRFA

SEQ ID NO: 37 - protein, 8 aa, synthetic construct
GFSFSTRS

SEQ ID NO: 38 - protein, 8 aa, synthetic construct
GFTFGDKA

SEQ ID NO: 39 - protein, 8 aa, synthetic construct
GFTFSRFA

SEQ ID NO: 40 - protein, 8 aa, synthetic construct
GFIFSTKA

SEQ ID NO: 41 - protein, 8 aa, synthetic construct
GLTFSIYA

SEQ ID NO: 42 - protein, 8 aa, synthetic construct
AFTFSRFA

SEQ ID NO: 43 - protein, 8 aa, synthetic construct
GLIFSNFA

SEQ ID NO: 44 - protein, 8 aa, synthetic construct
ISGSGGST

SEQ ID NO: 45 - protein, 8 aa, synthetic construct
ISGSGATT

SEQ ID NO: 46 - protein, 8 aa, synthetic construct
ISYEGRTQ

SEQ ID NO: 47 - protein, 8 aa, synthetic construct
ISGSGDTT

SEQ ID NO: 48 - protein, 8 aa, synthetic construct
ISGSGSNA

SEQ ID NO: 49 - protein, 8 aa, synthetic construct
ISGTGATT

SEQ ID NO: 50 - protein, 8 aa, synthetic construct
ISGSGSTV

SEQ ID NO: 51 - protein, 8 aa, synthetic construct
ISSSGTTS

SEQ ID NO: 52 - protein, 8 aa, synthetic construct
ISGSGLTS

SEQ ID NO: 53 - protein, 8 aa, synthetic construct
ISGTGYTT

SEQ ID NO: 54 - protein, 8 aa, synthetic construct
ISSSGSTM

SEQ ID NO: 55 - protein, 8 aa, synthetic construct
ISGSGSGT

SEQ ID NO: 56 - protein, 8 aa, synthetic construct
VSGSGSGT

SEQ ID NO: 57 - protein, 8 aa, synthetic construct
ISASGASR

SEQ ID NO: 58 - protein, 8 aa, synthetic construct
ISSSGTTT

SEQ ID NO: 59 - protein, 8 aa, synthetic construct
ISGTGDTT

SEQ ID NO: 60 - protein, 8 aa, synthetic construct
ISGSGATA

SEQ ID NO: 61 - protein, 8 aa, synthetic construct
ISGTGAST

SEQ ID NO: 62 - protein, 8 aa, synthetic construct
ISASGDTT

SEQ ID NO: 63 - protein, 8 aa, synthetic construct
ISSSASSK

SEQ ID NO: 64 - protein, 8 aa, synthetic construct
ISGTGSTR

SEQ ID NO: 65 - protein, 8 aa, synthetic construct
ISGSGATK

SEQ ID NO: 66 - protein, 8 aa, synthetic construct
ISSSGATT

SEQ ID NO: 67 - protein, 8 aa, synthetic construct
ISYEGSTT

SEQ ID NO: 68 - protein, 8 aa, synthetic construct
ISSSGSST

SEQ ID NO: 69 - protein, 8 aa, synthetic construct
ISATGAST

SEQ ID NO: 70 - protein, 8 aa, synthetic construct
IDATGRTI

SEQ ID NO: 71 - protein, 8 aa, synthetic construct
ISATGTSA

SEQ ID NO: 72 - protein, 8 aa, synthetic construct
ISGTAATT

SEQ ID NO: 73 - protein, 8 aa, synthetic construct
ISGSGSST

SEQ ID NO: 74 - protein, 8 aa, synthetic construct
ISYEGSTK

SEQ ID NO: 75 - protein, 8 aa, synthetic construct
ISSSGTTM

SEQ ID NO: 76 - protein, 8 aa, synthetic construct
ISSSATTM

SEQ ID NO: 77 - protein, 8 aa, synthetic construct
ISGSGSTM

SEQ ID NO: 78 - protein, 8 aa, synthetic construct
ISGSGFTT

SEQ ID NO: 79 - protein, 8 aa, synthetic construct
ISGRGDVT

SEQ ID NO: 80 - protein, 8 aa, synthetic construct
ISGSGDTM

SEQ ID NO: 81 - protein, 8 aa, synthetic construct
ISGSGKST

SEQ ID NO: 82 - protein, 8 aa, synthetic construct
ISGTGRST

SEQ ID NO: 83 - protein, 8 aa, synthetic construct
ISASGTTI

SEQ ID NO: 84 - protein, 8 aa, synthetic construct
ISGSGDTK

SEQ ID NO: 85 - protein, 8 aa, synthetic construct
ISSTGKST

SEQ ID NO: 86 - protein, 8 aa, synthetic construct
ISASAATT

SEQ ID NO: 87 - protein, 8 aa, synthetic construct
ISGPGMTS

SEQ ID NO: 88 - protein, 8 aa, synthetic construct
ISGSGTTK

SEQ ID NO: 89 - skipped

SEQ ID NO: 90 - protein, 10 aa, synthetic construct
ARKRWYGMDV

SEQ ID NO: 91-95 - skipped (5 entries)

SEQ ID NO: 96 - protein, 106 aa, synthetic construct
SYELTQPPSVSVAPGKTARITCGGNNIGSKSVHWYQQKPGQAPVLVVYDDSDRPSGIPERFSGSNSGNTATLTISRVEAGDEADYYCQVWDSRTYVFGTGTKLTVL

SEQ ID NO: 97 - protein, 105 aa, synthetic construct
SYELTQPPSVSVAPGKTARITCGGNNIGSKSVHWYQQKPGQAPVLVVYDDSDRPSGIPERFSGSNSGNTATLTISRVEAGDEADYYCQVWDRGMIFGTGTKLTVL

SEQ ID NO: 98 - protein, 105 aa, synthetic construct
SYELTQPPSVSVAPGKTARITCGGNNIVTKSVHWYQQKPGQAPVLVVYDDSDRPSGIPERFSGSNSGNTATLTISRVEAGDEADYYCQVWDRGMIFGTGTKLTVL

SEQ ID NO: 99 - protein, 106 aa, synthetic construct
SYELTQPPSVSVAPGKTARITCGGNNIGVKSVHWYQQKPGQAPVLVVYDDSDRPSGIPERFSGSNSGNTATLTISRVEAGDEADYYCQVWDSRTYVFGTGTKLTVL

SEQ ID NO: 100 - protein, 106 aa, synthetic construct
SYELTQPPSVSVAPGKTARITCGGNNIGVKSVHWYQQKPGQAPVLVVYDDSDRSPGIPERFSGSNSGNTATLTISRVEAGDEADYYCQVWDSRTYVFGTGTKLTVL

SEQ ID NO: 101 - protein, 105 aa, synthetic construct
SYELTQPPSVSVAPGKTARITCGGNNIGMKSVHWYQQKPGQAPVLVVYDDSDRPSGIPERFSGSNSGNTATLTISRVEAGDEADYYCQVWDRGMIFGTGTKLTVL

SEQ ID NO: 102 - protein, 105 aa, synthetic construct
SYELTQPPSVSVAPGKTARITCGGNNIGLKSVHWYQQKPGQAPVLVVYDDSDRPSGIPERFSGSNSGNTATLTISRVEAGDEADYYCQVWDRGMIFGTGTKLTVL

SEQ ID NO: 103 - protein, 105 aa, synthetic construct
SYELTQPPSVSVAPGKTARITCGGNNIGSKMVHWYQQKPGQAPVLVVYDDSDRPSGIPERFSGSNSGNTATLTISRVEAGDEADYYCQVWDRGMIFGTGTKLTVL

SEQ ID NO: 104 - protein, 105 aa, synthetic construct
SYELTQPPSVSVAPGKTARITCGGNNIGVKAVHWYQQKPGQAPVLVVYDDSDRPSGIPERFSGSNSGNTATLTISRVEAGDEADYYCQVWDRGMIFGTGTKLTVL

SEQ ID NO: 105 - protein, 106 aa, synthetic construct
SYELTQPPSVSVAPGKTARITCGGNNIGSKSVHWYQQKPGQAPVLVVYDDSDRPSGIPERFSGSNSGNTATLTISRVEAGDEADYYCQVWGRYQMIFGTGTKLTVL

SEQ ID NO: 106 - protein, 106 aa, synthetic construct
SYELTQPPSVSVAPGKTARITCGGNNIWGKSVHWYQQKPGQAPVLVVYDDIDRPSGIPERFSGSNSGNTATLTISRVEAGDEADYYCQVWDSRTYVFGTGTKLTVL

SEQ ID NO: 107 - protein, 106 aa, synthetic construct
SYELTQPPSVSVAPGKTARITCGGNDIGKKSVHWYQQKPGQAPVLVVYDDSDRPSGIPERFSGSNSGNTATLTISRVEAGDEADYYCQVWDSRTYVFGTGTKLTVL

SEQ ID NO: 108 - protein, 105 aa, synthetic construct
SYELTQPPSVSVAPGKTARITCGGNNIGVKSVHWYQQKPGQAPVLVVYDDSDRSPGIPERFSGSNSGNTATLTISRVEAGDEADYYCQVWDRGMIFGTGTKLTVL

SEQ ID NO: 109 - protein, 105 aa, synthetic construct
SYELTQPPSVSVAPGKTARITCGGNNIGSKSVHWYQQKPGQAPVLVVYDDIDRPAGIPERFSGSNSGNTATLTISRVEAGDEADYYCQVWDRGMIFGTGTKLTVL

SEQ ID NO: 110 - protein, 105 aa, synthetic construct
SYELTQPPSVSVAPGKTARITCGGNNIWGKSVHWYQQKPGQAPVLVVYDDIDRPSGIPERFSGSNSGNTATLTISRVEAGDEADYYCQVWDRGMIFGTGTKLTVL

SEQ ID NO: 111 - protein, 108 aa, synthetic construct
SYELTQPPSVSVAPGKTARITCGGNNIGSKSVHWYQQKPGQAPVLVVYDDSDRPSGIPERFSGSNSGNTATLTISRVEAGDEADYYCQVWASTTAPSVFGTGTKLTVL

SEQ ID NO: 112 - protein, 106 aa, synthetic construct
SYELTQPPSVSVAPGKTARITCGGNNIVTKSVHWYQQKPGQAPVLVVYDDIDRPSGIPERFSGSNSGNTATLTISRVEAGDEADYYCQVWDSRTYVFGTGTKLTVL

SEQ ID NO: 113 - protein, 105 aa, synthetic construct
SYELTQPPSVSVAPGKTARITCGGNDIGKKSVHWYQQKPGQAPVLVVYDDSDRPSGIPERFSGSNSGNTATLTISRVEAGDEADYYCQVWDRGMIFGTGTKLTVL

SEQ ID NO: 114 - protein, 106 aa, synthetic construct
SYELTQPPSVSVAPGKTARITCGGNNIGSKSVHWYQQKPGQAPVLVVYDDSDRPSGIPERFSGSNSGNTATLTISRVEAGDEADYYCQVWDSRSVVFGTGTKLTVL

SEQ ID NO: 115 - protein, 105 aa, synthetic construct
SYELTQPPSVSVAPGKTARITCGGNNIGYKSVHWYQQKPGQAPVLVVYDDSDRPSGIPERFSGSNSGNTATLTISRVEAGDEADYYCQVWDRGMIFGTGTKLTVL

SEQ ID NO: 116 - protein, 106 aa, synthetic construct
SYELTQPPSVSVAPGKTARITCGGNKLGDKWVHWYQQKPGQAPVLVVYEDVDRPSGIPERFSGSNSGNTATLTISRVEAGDEADYYCQVWDSRTYVFGTGTKLTVL

SEQ ID NO: 117 - protein, 106 aa, synthetic construct
SYELTQPPSVSVAPGKTARITCGGNNIGYKSVHWYQQKPGQAPVLVVYDDSDRPSGIPERFSGSNSGNTATLTISRVEAGDEADYYCQVWDSRTYVFGTGTKLTVL

SEQ ID NO: 118 - protein, 105 aa, synthetic construct
SYELTQPPSVSVAPGKTARITCGGNNIGFKSVHWYQQKPGQAPVLVVYDDSDRPSGIPERFSGSNSGNTATLTISRVEAGDEADYYCQVWDRGMIFGTGTKLTVL

SEQ ID NO: 119 - protein, 117 aa, synthetic construct
EVQLVESGGGVVQPGRSLRLSCAASGFTFSSYSMNWVRQAPGKGLEWVSAISGSGGSTYYADSVKGRFTISRDNSKNTLYLQMNSLRAEDTAVYYCARKRWYGMDVWGQGTTVTVSS

SEQ ID NO: 120 - protein, 117 aa, synthetic construct
EVQLVESGGGVVQPGRSLRLSCAASGFTFSNKAMNWVRQAPGKGLEWVSAISGSGATTYYADSVKGRFTISRDNSKNTLYLQMNSLRAEDTAVYYCARKRWYGMDVWGQGTTVTVSS

SEQ ID NO: 121 - protein, 117 aa, synthetic construct
EVQLVESGGGVVQPGRSLRLSCAASGFTFTSRAMNWVRQAPGKGLEWVSAISYEGRTQYYADSVKGRFTISRDNSKNTLYLQMNSLRAEDTAVYYCARKRWYGMDVWGQGTTVTVSS

SEQ ID NO: 122 - protein, 117 aa, synthetic construct
EVQLVESGGGVVQPGRSLRLSCAASGFTFSSRSMNWVRQAPGKGLEWVSAISGSGDTTYYADSVKGRFTISRDNSKNTLYLQMNSLRAEDTAVYYCARKRWYGMDVWGQGTTVTVSS

SEQ ID NO: 123 - protein, 117 aa, synthetic construct
EVQLVESGGGVVQPGRSLRLSCAASGFTYSSRAMNWVRQAPGKGLEWVSAISGSGSNAYYADSVKGRFTISRDNSKNTLYLQMNSLRAEDTAVYYCARKRWYGMDVWGQGTTVTVSS

SEQ ID NO: 124 - protein, 117 aa, synthetic construct
EVQLVESGGGVVQPGRSLRLSCAASGFTFGDRAMNWVRQAPGKGLEWVSAISGTGATTYYADSVKGRFTISRDNSKNTLYLQMNSLRAEDTAVYYCARKRWYGMDVWGQGTTVTVSS

SEQ ID NO: 125 - protein, 117 aa, synthetic construct
EVQLVESGGGVVQPGRSLRLSCAASGFTFSSRSMNWVRQAPGKGLEWVSAISGSGSTVYYADSVKGRFTISRDNSKNTLYLQMNSLRAEDTAVYYCARKRWYGMDVWGQGTTVTVSS

SEQ ID NO: 126 - protein, 117 aa, synthetic construct
EVQLVESGGGVVQPGRSLRLSCAASGFTFGDRAMNWVRQAPGKGLEWVSAISSSGTTSYYADSVKGRFTISRDNSKNTLYLQMNSLRAEDTAVYYCARKRWYGMDVWGQGTTVTVSS

SEQ ID NO: 127 - protein, 117 aa, synthetic construct
EVQLVESGGGVVQPGRSLRLSCAASGFTFSSRSMNWVRQAPGKGLEWVSAISGSGLTSYYADSVKGRFTISRDNSKNTLYLQMNSLRAEDTAVYYCARKRWYGMDVWGQGTTVTVSS

SEQ ID NO: 128 - protein, 117 aa, synthetic construct
EVQLVESGGGVVQPGRSLRLSCAASGFTFSSRSMNWVRQAPGKGLEWVSAISGTGYTTYYADSVKGRFTISRDNSKNTLYLQMNSLRAEDTAVYYCARKRWYGMDVWGQGTTVTVSS

SEQ ID NO: 129 - protein, 117 aa, synthetic construct
EVQLVESGGGVVQPGRSLRLSCAASGFTFDNRAMNWVRQAPGKGLEWVSAISSSGSTMYYADSVKGRFTISRDNSKNTLYLQMNSLRAEDTAVYYCARKRWYGMDVWGQGTTVTVSS

SEQ ID NO: 130 - protein, 117 aa, synthetic construct
EVQLVESGGGVVQPGRSLRLSCAASGFTFSDRAMNWVRQAPGKGLEWVSAISYEGRTQYYADSVKGRFTISRDNSKNTLYLQMNSLRAEDTAVYYCARKRWYGMDVWGQGTTVTVSS

SEQ ID NO: 131 - protein, 117 aa, synthetic construct
EVQLVESGGGVVQPGRSLRLSCAASGFTFGDRAMNWVRQAPGKGLEWVSAISGSGSGTYYADSVKGRFTISRDNSKNTLYLQMNSLRAEDTAVYYCARKRWYGMDVWGQGTTVTVSS

SEQ ID NO: 132 - protein, 117 aa, synthetic construct
EVQLVESGGGVVQPGRSLRLSCAASGFSFSSRAMNWVRQAPGKGLEWVSAVSGSGSGTYYADSVKGRFTISRDNSKNTLYLQMNSLRAEDTAVYYCARKRWYGMDVWGQGTTVTVSS

SEQ ID NO: 133 - protein, 117 aa, synthetic construct
EVQLVESGGGVVQPGRSLRLSCAASGLTFSSRAMNWVRQAPGKGLEWVSAISGTGATTYYADSVKGRFTISRDNSKNTLYLQMNSLRAEDTAVYYCARKRWYGMDVWGQGTTVTVSS

SEQ ID NO: 134 - protein, 117 aa, synthetic construct
EVQLVESGGGVVQPGRSLRLSCAASGFTFGDRAMNWVRQAPGKGLEWVSAISGSGATTYYADSVKGRFTISRDNSKNTLYLQMNSLRAEDTAVYYCARKRWYGMDVWGQGTTVTVSS

SEQ ID NO: 135 - protein, 117 aa, synthetic construct
EVQLVESGGGVVQPGRSLRLSCAASGFTFSDRAMNWVRQAPGKGLEWVSAISASGASRYYADSVKGRFTISRDNSKNTLYLQMNSLRAEDTAVYYCARKRWYGMDVWGQGTTVTVSS

SEQ ID NO: 136 - protein, 117 aa, synthetic construct
EVQLVESGGGVVQPGRSLRLSCAASGFTYSSRAMNWVRQAPGKGLEWVSAISSSGTTTYYADSVKGRFTISRDNSKNTLYLQMNSLRAEDTAVYYCARKRWYGMDVWGQGTTVTVSS

SEQ ID NO: 137 - protein, 117 aa, synthetic construct
EVQLVESGGGVVQPGRSLRLSCAASGFTFSTRAMNWVRQAPGKGLEWVSAISGTGDTTYYADSVKGRFTISRDNSKNTLYLQMNSLRAEDTAVYYCARKRWYGMDVWGQGTTVTVSS

SEQ ID NO: 138 - protein, 117 aa, synthetic construct
EVQLVESGGGVVQPGRSLRLSCAASGFTFTSRAMNWVRQAPGKGLEWVSAISGSGATAYYADSVKGRFTISRDNSKNTLYLQMNSLRAEDTAVYYCARKRWYGMDVWGQGTTVTVSS

SEQ ID NO: 139 - protein, 117 aa, synthetic construct
EVQLVESGGGVVQPGRSLRLSCAASGFTFSDRAMNWVRQAPGKGLEWVSAISGTGASTYYADSVKGRFTISRDNSKNTLYLQMNSLRAEDTAVYYCARKRWYGMDVWGQGTTVTVSS

SEQ ID NO: 140 - protein, 117 aa, synthetic construct
EVQLVESGGGVVQPGRSLRLSCAASGFTFSSRSMNWVRQAPGKGLEWVSAISGSGATKYYADSVKGRFTISRDNSKNTLYLQMNSLRAEDTAVYYCARKRWYGMDVWGQGTTVTVSS

SEQ ID NO: 141 - protein, 117 aa, synthetic construct
EVQLVESGGGVVQPGRSLRLSCAASGFSFSSRAMNWVRQAPGKGLEWVSAISSSGATTYYADSVKGRFTISRDNSKNTLYLQMNSLRAEDTAVYYCARKRWYGMDVWGQGTTVTVSS

SEQ ID NO: 142 - protein, 117 aa, synthetic construct
EVQLVESGGGVVQPGRSLRLSCAASGFTFSRQAMNWVRQAPGKGLEWVSAISYEGRTQYYADSVKGRFTISRDNSKNTLYLQMNSLRAEDTAVYYCARKRWYGMDVWGQGTTVTVSS

SEQ ID NO: 143 - protein, 117 aa, synthetic construct
EVQLVESGGGVVQPGRSLRLSCAASGLTFSRFAMNWVRQAPGKGLEWVSAISSSGTTSYYADSVKGRFTISRDNSKNTLYLQMNSLRAEDTAVYYCARKRWYGMDVWGQGTTVTVSS

SEQ ID NO: 144 - protein, 117 aa, synthetic construct
EVQLVESGGGVVQPGRSLRLSCAASGFTFSSRAMNWVRQAPGKGLEWVSAISYEGSTTYYADSVKGRFTISRDNSKNTLYLQMNSLRAEDTAVYYCARKRWYGMDVWGQGTTVTVSS

SEQ ID NO: 145 - protein, 117 aa, synthetic construct
EVQLVESGGGVVQPGRSLRLSCAASGFTFSSRAMNWVRQAPGKGLEWVSAISASGDTTYYADSVKGRFTISRDNSKNTLYLQMNSLRAEDTAVYYCARKRWYGMDVWGQGTTVTVSS

SEQ ID NO: 146 - protein, 117 aa, synthetic construct
EVQLVESGGGVVQPGRSLRLSCAASGFTFSDRAMNWVRQAPGKGLEWVSAISSSASSKYYADSVKGRFTISRDNSKNTLYLQMNSLRAEDTAVYYCARKRWYGMDVWGQGTTVTVSS

SEQ ID NO: 147 - protein, 117 aa, synthetic construct
EVQLVESGGGVVQPGRSLRLSCAASGFTFGDRAMNWVRQAPGKGLEWVSAISGTGSTRYYADSVKGRFTISRDNSKNTLYLQMNSLRAEDTAVYYCARKRWYGMDVWGQGTTVTVSS

SEQ ID NO: 148 - protein, 117 aa, synthetic construct
EVQLVESGGGVVQPGRSLRLSCAASGFTFSSRAMNWVRQAPGKGLEWVSAISSSGTTSYYADSVKGRFTISRDNSKNTLYLQMNSLRAEDTAVYYCARKRWYGMDVWGQGTTVTVSS

SEQ ID NO: 149 - protein, 117 aa, synthetic construct
EVQLVESGGGVVQPGRSLRLSCAASGFSFSTRSMNWVRQAPGKGLEWVSAISGSGATAYYADSVKGRFTISRDNSKNTLYLQMNSLRAEDTAVYYCARKRWYGMDVWGQGTTVTVSS

SEQ ID NO: 150 - protein, 117 aa, synthetic construct
EVQLVESGGGVVQPGRSLRLSCAASGFTFSDRAMNWVRQAPGKGLEWVSAISATGASTYYADSVKGRFTISRDNSKNTLYLQMNSLRAEDTAVYYCARKRWYGMDVWGQGTTVTVSS

SEQ ID NO: 151 - protein, 117 aa, synthetic construct
EVQLVESGGGVVQPGRSLRLSCAASGFSFSSRAMNWVRQAPGKGLEWVSAIDATGRTIYYADSVKGRFTISRDNSKNTLYLQMNSLRAEDTAVYYCARKRWYGMDVWGQGTTVTVSS

SEQ ID NO: 152 - protein, 117 aa, synthetic construct
EVQLVESGGGVVQPGRSLRLSCAASGFTFSSRSMNWVRQAPGKGLEWVSAISGTGSTRYYADSVKGRFTISRDNSKNTLYLQMNSLRAEDTAVYYCARKRWYGMDVWGQGTTVTVSS

SEQ ID NO: 153 - protein, 117 aa, synthetic construct
EVQLVESGGGVVQPGRSLRLSCAASGFTYSSRAMNWVRQAPGKGLEWVSAISATGTSAYYADSVKGRFTISRDNSKNTLYLQMNSLRAEDTAVYYCARKRWYGMDVWGQGTTVTVSS

SEQ ID NO: 154 - protein, 117 aa, synthetic construct
EVQLVESGGGVVQPGRSLRLSCAASGFTFSTRAMNWVRQAPGKGLEWVSAISGSGSNAYYADSVKGRFTISRDNSKNTLYLQMNSLRAEDTAVYYCARKRWYGMDVWGQGTTVTVSS

SEQ ID NO: 155 - protein, 117 aa, synthetic construct
EVQLVESGGGVVQPGRSLRLSCAASGFTFSDRAMNWVRQAPGKGLEWVSAISGTAATTYYADSVKGRFTISRDNSKNTLYLQMNSLRAEDTAVYYCARKRWYGMDVWGQGTTVTVSS

SEQ ID NO: 156 - protein, 117 aa, synthetic construct
EVQLVESGGGVVQPGRSLRLSCAASGFTFSTRAMNWVRQAPGKGLEWVSAISSSGSSTYYADSVKGRFTISRDNSKNTLYLQMNSLRAEDTAVYYCARKRWYGMDVWGQGTTVTVSS

SEQ ID NO: 157 - protein, 117 aa, synthetic construct
EVQLVESGGGVVQPGRSLRLSCAASGFTYSSRAMNWVRQAPGKGLEWVSAISGSGATKYYADSVKGRFTISRDNSKNTLYLQMNSLRAEDTAVYYCARKRWYGMDVWGQGTTVTVSS

SEQ ID NO: 158 - protein, 117 aa, synthetic construct
EVQLVESGGGVVQPGRSLRLSCAASGFTFSSRSMNWVRQAPGKGLEWVSAISGSGSSTYYADSVKGRFTISRDNSKNTLYLQMNSLRAEDTAVYYCARKRWYGMDVWGQGTTVTVSS

SEQ ID NO: 159 - protein, 117 aa, synthetic construct
EVQLVESGGGVVQPGRSLRLSCAASGFTFSDRAMNWVRQAPGKGLEWVSAISSSATTMYYADSVKGRFTISRDNSKNTLYLQMNSLRAEDTAVYYCARKRWYGMDVWGQGTTVTVSS

SEQ ID NO: 160 - protein, 117 aa, synthetic construct
EVQLVESGGGVVQPGRSLRLSCAASGFTFGDKAMNWVRQAPGKGLEWVSAISSSGTTMYYADSVKGRFTISRDNSKNTLYLQMNSLRAEDTAVYYCARKRWYGMDVWGQGTTVTVSS

SEQ ID NO: 161 - protein, 117 aa, synthetic construct
EVQLVESGGGVVQPGRSLRLSCAASGFTFSRFAMNWVRQAPGKGLEWVSAISSSGSTAYYADSVKGRFTISRDNSKNTLYLQMNSLRAEDTAVYYCARKRWYGMDVWGQGTTVTVSS

SEQ ID NO: 162 - protein, 117 aa, synthetic construct
EVQLVESGGGVVQPGRSLRLSCAASGFIFSTKAMNWVRQAPGKGLEWVSAISSSGATTYYADSVKGRFTISRDNSKNTLYLQMNSLRAEDTAVYYCARKRWYGMDVWGQGTTVTVSS

SEQ ID NO: 163 - protein, 117 aa, synthetic construct
EVQLVESGGGVVQPGRSLRLSCAASGFTFSNKAMNWVRQAPGKGLEWVSAISYEGSTKYYADSVKGRFTISRDNSKNTLYLQMNSLRAEDTAVYYCARKRWYGMDVWGQGTTVTVSS

SEQ ID NO: 164 - protein, 117 aa, synthetic construct
EVQLVESGGGVVQPGRSLRLSCAASGFTFSSRSMNWVRQAPGKGLEWVSAISSSGTTMYYADSVKGRFTISRDNSKNTLYLQMNSLRAEDTAVYYCARKRWYGMDVWGQGTTVTVSS

SEQ ID NO: 165 - protein, 117 aa, synthetic construct
EVQLVESGGGVVQPGRSLRLSCAASGFTFGDRAMNWVRQAPGKGLEWVSAISSSGATTYYADSVKGRFTISRDNSKNTLYLQMNSLRAEDTAVYYCARKRWYGMDVWGQGTTVTVSS

SEQ ID NO: 166 - protein, 117 aa, synthetic construct
EVQLVESGGGVVQPGRSLRLSCAASGFTFSSRAMNWVRQAPGKGLEWVSAISGSGSTMYYADSVKGRFTISRDNSKNTLYLQMNSLRAEDTAVYYCARKRWYGMDVWGQGTTVTVSS

SEQ ID NO: 167 - protein, 117 aa, synthetic construct
EVQLVESGGGVVQPGRSLRLSCAASGFTFSSRSMNWVRQAPGKGLEWVSAISGRGDVTYYADSVKGRFTISRDNSKNTLYLQMNSLRAEDTAVYYCARKRWYGMDVWGQGTTVTVSS

SEQ ID NO: 168 - protein, 117 aa, synthetic construct
EVQLVESGGGVVQPGRSLRLSCAASGFTFDNRAMNWVRQAPGKGLEWVSAISYEGRTQYYADSVKGRFTISRDNSKNTLYLQMNSLRAEDTAVYYCARKRWYGMDVWGQGTTVTVSS

SEQ ID NO: 169 - protein, 117 aa, synthetic construct
EVQLVESGGGVVQPGRSLRLSCAASGFTFSSRSMNWVRQAPGKGLEWVSAISGSGDTMYYADSVKGRFTISRDNSKNTLYLQMNSLRAEDTAVYYCARKRWYGMDVWGQGTTVTVSS

SEQ ID NO: 170 - protein, 117 aa, synthetic construct
EVQLVESGGGVVQPGRSLRLSCAASGLTFSIYAMNWVRQAPGKGLEWVSAISGSGATAYYADSVKGRFTISRDNSKNTLYLQMNSLRAEDTAVYYCARKRWYGMDVWGQGTTVTVSS

SEQ ID NO: 171 - protein, 117 aa, synthetic construct
EVQLVESGGGVVQPGRSLRLSCAASGLTFSSRAMNWVRQAPGKGLEWVSAISSSGTTTYYADSVKGRFTISRDNSKNTLYLQMNSLRAEDTAVYYCARKRWYGMDVWGQGTTVTVSS

SEQ ID NO: 172 - protein, 117 aa, synthetic construct
EVQLVESGGGVVQPGRSLRLSCAASGFTFSSRAMNWVRQAPGKGLEWVSAISSSGSTAYYADSVKGRFTISRDNSKNTLYLQMNSLRAEDTAVYYCARKRWYGMDVWGQGTTVTVSS

SEQ ID NO: 173 - protein, 117 aa, synthetic construct
EVQLVESGGGVVQPGRSLRLSCAASGFTFSDRAMNWVRQAPGKGLEWVSAISGSGKSTYYADSVKGRFTISRDNSKNTLYLQMNSLRAEDTAVYYCARKRWYGMDVWGQGTTVTVSS

SEQ ID NO: 174 - protein, 117 aa, synthetic construct
EVQLVESGGGVVQPGRSLRLSCAASGFTFSSRAMNWVRQAPGKGLEWVSAISGSGATAYYADSVKGRFTISRDNSKNTLYLQMNSLRAEDTAVYYCARKRWYGMDVWGQGTTVTVSS

SEQ ID NO: 175 - protein, 117 aa, synthetic construct
EVQLVESGGGVVQPGRSLRLSCAASGFTFSDRAMNWVRQAPGKGLEWVSAISGSGFTTYYADSVKGRFTISRDNSKNTLYLQMNSLRAEDTAVYYCARKRWYGMDVWGQGTTVTVSS

SEQ ID NO: 176 - protein, 117 aa, synthetic construct
EVQLVESGGGVVQPGRSLRLSCAASGFIFSTKAMNWVRQAPGKGLEWVSAISGSGATAYYADSVKGRFTISRDNSKNTLYLQMNSLRAEDTAVYYCARKRWYGMDVWGQGTTVTVSS

SEQ ID NO: 177 - protein, 117 aa, synthetic construct
EVQLVESGGGVVQPGRSLRLSCAASGFTFDNRAMNWVRQAPGKGLEWVSAISGSGDTKYYADSVKGRFTISRDNSKNTLYLQMNSLRAEDTAVYYCARKRWYGMDVWGQGTTVTVSS

SEQ ID NO: 178 - protein, 117 aa, synthetic construct
EVQLVESGGGVVQPGRSLRLSCAASGFTFTSRAMNWVRQAPGKGLEWVSAISASGDTTYYADSVKGRFTISRDNSKNTLYLQMNSLRAEDTAVYYCARKRWYGMDVWGQGTTVTVSS

SEQ ID NO: 179 - protein, 117 aa, synthetic construct
EVQLVESGGGVVQPGRSLRLSCAASAFTFSRFAMNWVRQAPGKGLEWVSAISSSGSTAYYADSVKGRFTISRDNSKNTLYLQMNSLRAEDTAVYYCARKRWYGMDVWGQGTTVTVSS

SEQ ID NO: 180 - protein, 117 aa, synthetic construct
EVQLVESGGGVVQPGRSLRLSCAASGFTFDNRAMNWVRQAPGKGLEWVSAISSTGKSTYYADSVKGRFTISRDNSKNTLYLQMNSLRAEDTAVYYCARKRWYGMDVWGQGTTVTVSS

SEQ ID NO: 181 - protein, 117 aa, synthetic construct
EVQLVESGGGVVQPGRSLRLSCAASGFIFSTKAMNWVRQAPGKGLEWVSAISYEGRTQYYADSVKGRFTISRDNSKNTLYLQMNSLRAEDTAVYYCARKRWYGMDVWGQGTTVTVSS

SEQ ID NO: 182 - protein, 117 aa, synthetic construct
EVQLVESGGGVVQPGRSLRLSCAASGFTYSSRAMNWVRQAPGKGLEWVSAISASGDTTYYADSVKGRFTISRDNSKNTLYLQMNSLRAEDTAVYYCARKRWYGMDVWGQGTTVTVSS

SEQ ID NO: 183 - protein, 117 aa, synthetic construct
EVQLVESGGGVVQPGRSLRLSCAASGFTFSDRAMNWVRQAPGKGLEWVSAISASAATTYYADSVKGRFTISRDNSKNTLYLQMNSLRAEDTAVYYCARKRWYGMDVWGQGTTVTVSS

SEQ ID NO: 184 - protein, 117 aa, synthetic construct
EVQLVESGGGVVQPGRSLRLSCAASGFTFGDRAMNWVRQAPGKGLEWVSAISGTGRSTYYADSVKGRFTISRDNSKNTLYLQMNSLRAEDTAVYYCARKRWYGMDVWGQGTTVTVSS

SEQ ID NO: 185 - protein, 117 aa, synthetic construct
EVQLVESGGGVVQPGRSLRLSCAASGFTFSSRAMNWVRQAPGKGLEWVSAISSSGTTMYYADSVKGRFTISRDNSKNTLYLQMNSLRAEDTAVYYCARKRWYGMDVWGQGTTVTVSS

SEQ ID NO: 186 - protein, 117 aa, synthetic construct
EVQLVESGGGVVQPGRSLRLSCAASGFTFSSRSMNWVRQAPGKGLEWVSAISASGTTIYYADSVKGRFTISRDNSKNTLYLQMNSLRAEDTAVYYCARKRWYGMDVWGQGTTVTVSS

SEQ ID NO: 187 - protein, 117 aa, synthetic construct
EVQLVESGGGVVQPGRSLRLSCAASGFTFSSRAMNWVRQAPGKGLEWVSAISSSGTTTYYADSVKGRFTISRDNSKNTLYLQMNSLRAEDTAVYYCARKRWYGMDVWGQGTTVTVSS

SEQ ID NO: 188 - protein, 117 aa, synthetic construct
EVQLVESGGGVVQPGRSLRLSCAASGLIFSNFAMNWVRQAPGKGLEWVSAISSSGTTMYYADSVKGRFTISRDNSKNTLYLQMNSLRAEDTAVYYCARKRWYGMDVWGQGTTVTVSS

SEQ ID NO: 189 - protein, 117 aa, synthetic construct
EVQLVESGGGVVQPGRSLRLSCAASGFTFSSRAMNWVRQAPGKGLEWVSAISGPGMTSYYADSVKGRFTISRDNSKNTLYLQMNSLRAEDTAVYYCARKRWYGMDVWGQGTTVTVSS

SEQ ID NO: 190 - protein, 117 aa, synthetic construct
EVQLVESGGGVVQPGRSLRLSCAASGFTFSDRAMNWVRQAPGKGLEWVSAISGSGTTKYYADSVKGRFTISRDNSKNTLYLQMNSLRAEDTAVYYCARKRWYGMDVWGQGTTVTVSS

SEQ ID NO: 191 - protein, 282 aa, synthetic construct
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

(19) United States
(12) Patent Application Publication
(10) Pub. No.: US 2024/0309095 A1
(43) Pub. Date: Sep. 19, 2024

(54) ANTI-B7-H4 ANTIBODIES AND USES THEREOF

(71) Applicants: Incyte Corporation, Wilmington, DE (US); SPECIFICA INC., Santa Fe, NM (US)

(72) Inventors: Fortunato Ferrara, Sante Fe, NM (US); Michael Frank Erasmus, Santa Fe, NM (US); Andre Azevedo Reis Teixeira, Sante Fe, NM (US); Andrew Raymon Morton Bradbury, Santa Fe, NM (US)

(21) Appl. No.: 18/576,936

(22) PCT Filed: Jul. 7, 2022
(86) PCT No.: PCT/US2022/036371

§ 371 Date: Jan. 5, 2024

Related U.S. Application Data
(60) Provisional application No. 63/323,632, filed on Mar. 25, 2022.
(60) Provisional application No. 63/218,919, filed on Jul. 7, 2021.

(51) Int. Cl. C07K 16/28; A61K 47/68

(52) U.S. Cl. CPC C07K 16/2827; A61K 47/6849; C07K 2317/565; C07K 2317/92

(57) ABSTRACT

Anti-B7-H4 antibodies are disclosed. Also disclosed are related nucleic acids, vectors, cells, kits, and pharmaceutical compositions. Methods of treating cancer with the anti-B7-H4 antibodies are also disclosed.

### CROSS-REFERENCE TO RELATED APPLICATIONS

This application claims the right of priority to U.S. Provisional Appl. No. 63/218,919, filed Jul. 7, 2021, and U.S. Provisional Appl. No. 63/323,632, filed Mar. 25, 2022. The contents of the prior applications are incorporated by reference herein in their entirety.

### BACKGROUND

B7-H4 (also known as B7x, B7S1, or VTCN1) is a highly evolutionarily conserved transmembrane protein. B7-H4 mRNA is widely expressed, but the presence of B7-H4 protein on the surface of normal cells is limited. Unlike normal tissues, high levels of B7-H4 protein expression have been reported on a variety of human tumors including ovarian cancer, breast cancer, endometrial cancer, bladder cancer, esophageal cancer, oral squamous cell carcinoma, brain cancer, prostate cancer, pancreatic cancer, cervical cancer, skin cancer, lung cancer, gastric cancer, and renal cell carcinoma. Moreover, B7-H4 expression correlates with various pathological features including increased tumor size, decreased patient survival, and decreased number of tumor-infiltrating T cells. Thus, there is a need for therapeutics targeting B7-H4 in cancer.

### SUMMARY

The present disclosure is based, at least in part, on the development of antibodies having binding affinity and specificity to B7-H4.

Accordingly, aspects of the present disclosure provide an antibody that binds to human B7-H4, wherein the antibody comprises a light chain variable region (VL) comprising a VL CDR1, a VL CDR2, and a VL CDR3, wherein the VL CDR1 comprises the amino acid sequence of any one of SEQ ID NOs:1-12; the VL CDR2 comprises the amino acid sequence of any one of SEQ ID NOs:13-17; the VL CDR3 comprises the amino acid sequence of any one of SEQ ID NOs:18-22; the VH CDR1 comprises the amino acid sequence of any one of SEQ ID NOs:23-43; the VH CDR2 comprises the amino acid sequence of any one of SEQ ID NOs:44-89; and the VH CDR3 comprises the amino acid sequence of SEQ ID NO:90.

In some embodiments, the VL CDR1, the VL CDR2, and the VL CDR3 each correspond to the VL CDRs set forth in Table 4 for a single VL clone, and wherein the VH CDR1, the VH CDR2, and the VH CDR3 each correspond to the VH CDRs set forth in Table 4 for a single VH clone.

In some embodiments, the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:23; the VH CDR2 comprises the amino acid sequence of SEQ ID NO:44; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:24; the VH CDR2 comprises the amino acid sequence of SEQ ID NO:45; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:25; the VH CDR2 comprises the amino acid sequence of SEQ ID NO:46; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:47; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:27; the VH CDR2 comprises the amino acid sequence of SEQ ID NO:48; and the VH CDR3 comprises the amino acid sequence of SEQ ID NO:90;
the VL CDR1 comprises the amino acid sequence of SEQ ID NO:2; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:28; the VH CDR2 comprises the amino acid sequence of SEQ ID NO:49; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:50; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:28; the VH CDR2 comprises the amino acid sequence of SEQ ID NO:51; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:52; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:53; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:50; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:29; the VH CDR2 comprises the amino acid sequence of SEQ ID NO:54; and the VH CDR3 comprises the amino acid sequence of SEQ ID NO:90;
the VL CDR1 comprises the amino acid sequence of SEQ ID NO:3; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:30; the VH CDR2 comprises the amino acid sequence of SEQ ID NO:46; and the VH CDR3 comprises the amino acid sequence of SEQ ID NO:90;
the VL CDR1 comprises the amino acid sequence of SEQ ID NO:3; the VL CDR2 comprises the amino acid sequence of SEQ ID NO:14; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:50; and the VH CDR3 comprises the amino acid sequence of SEQ ID NO:90;
the VL CDR1 comprises the amino acid sequence of SEQ ID NO:4; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:28; the VH CDR2 comprises the amino acid sequence of SEQ ID NO:55; and the VH CDR3 comprises the amino acid sequence of SEQ ID NO:90;
the VL CDR1 comprises the amino acid sequence of SEQ ID NO:5; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:31; the VH CDR2 comprises the amino acid sequence of SEQ ID NO:56; and the VH CDR3 comprises the amino acid sequence of SEQ ID NO:90;
the VL CDR1 comprises the amino acid sequence of SEQ ID NO:6; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:32; the VH CDR2 comprises the amino acid sequence of SEQ ID NO:49; and the VH CDR3 comprises the amino acid sequence of SEQ ID NO:90;
the VL CDR1 comprises the amino acid sequence of SEQ ID NO:7; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:24; the VH CDR2 comprises the amino acid sequence of SEQ ID NO:45; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:28; the VH CDR2 comprises the amino acid sequence of SEQ ID NO:45; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:57; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:27; the VH CDR2 comprises the amino acid sequence of SEQ ID NO:58; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:20; the VH CDR1 comprises the amino acid sequence of SEQ ID NO:33; the VH CDR2 comprises the amino acid sequence of SEQ ID NO:59; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:25; the VH CDR2 comprises the amino acid sequence of SEQ ID NO:60; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:61; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:29; the VH CDR2 comprises the amino acid sequence of SEQ ID NO:54; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:62; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:63; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:28; the VH CDR2 comprises the amino acid sequence of SEQ ID NO:64; and the VH CDR3 comprises the amino acid sequence of SEQ ID NO:90;
the VL CDR1 comprises the amino acid sequence of SEQ ID NO:8; the VL CDR2 comprises the amino acid sequence of SEQ ID NO:15; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:50; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:65; and the VH CDR3 comprises the amino acid sequence of SEQ ID NO:90;
the VL CDR1 comprises the amino acid sequence of SEQ ID NO:9; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:31; the VH CDR2 comprises the amino acid sequence of SEQ ID NO:66; and the VH CDR3 comprises the amino acid sequence of SEQ ID NO:90;
the VL CDR1 comprises the amino acid sequence of SEQ ID NO:3; the VL CDR2 comprises the amino acid sequence of SEQ ID NO:14; the VL CDR3 comprises the amino acid sequence of SEQ ID NO:19; the VH CDR1 comprises the amino acid sequence of SEQ ID NO:35; the VH CDR2 comprises the amino acid sequence of SEQ ID NO:46; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:36; the VH CDR2 comprises the amino acid sequence of SEQ ID NO:51; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:67; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:51; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:33; the VH CDR2 comprises the amino acid sequence of SEQ ID NO:68; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:21; the VH CDR1 comprises the amino acid sequence of SEQ ID NO:27; the VH CDR2 comprises the amino acid sequence of SEQ ID NO:65; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:37; the VH CDR2 comprises the amino acid sequence of SEQ ID NO:60; and the VH CDR3 comprises the amino acid sequence of SEQ ID NO:90;
the VL CDR1 comprises the amino acid sequence of SEQ ID NO:4; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:69; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:16; the VL CDR3 comprises the amino acid sequence of SEQ ID NO:19; the VH CDR1 comprises the amino acid sequence of SEQ ID NO:31; the VH CDR2 comprises the amino acid sequence of SEQ ID NO:70; and the VH CDR3 comprises the amino acid sequence of SEQ ID NO:90;
the VL CDR1 comprises the amino acid sequence of SEQ ID NO:8; the VL CDR2 comprises the amino acid sequence of SEQ ID NO:15; the VL CDR3 comprises the amino acid sequence of SEQ ID NO:19; the VH CDR1 comprises the amino acid sequence of SEQ ID NO:33; the VH CDR2 comprises the amino acid sequence of SEQ ID NO:59; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:64; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:27; the VH CDR2 comprises the amino acid sequence of SEQ ID NO:71; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:33; the VH CDR2 comprises the amino acid sequence of SEQ ID NO:48; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:72; and the VH CDR3 comprises the amino acid sequence of SEQ ID NO:90;
the VL CDR1 comprises the amino acid sequence of SEQ ID NO:2; the VL CDR2 comprises the amino acid sequence of SEQ ID NO:15; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:73; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:24; the VH CDR2 comprises the amino acid sequence of SEQ ID NO:74; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:75; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:28; the VH CDR2 comprises the amino acid sequence of SEQ ID NO:66; and the VH CDR3 comprises the amino acid sequence of SEQ ID NO:90;
the VL CDR1 comprises the amino acid sequence of SEQ ID NO:9; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:76; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:38; the VH CDR2 comprises the amino acid sequence of SEQ ID NO:75; and the VH CDR3 comprises the amino acid sequence of SEQ ID NO:90;
the VL CDR1 comprises the amino acid sequence of SEQ ID NO:8; the VL CDR2 comprises the amino acid sequence of SEQ ID NO:15; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:65; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:39; the VH CDR2 comprises the amino acid sequence of SEQ ID NO:76; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:40; the VH CDR2 comprises the amino acid sequence of SEQ ID NO:66; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:77; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:60; and the VH CDR3 comprises the amino acid sequence of SEQ ID NO:90;
the VL CDR1 comprises the amino acid sequence of SEQ ID NO.1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:78; and the VH CDR3 comprises the amino acid sequence of SEQ ID NO:90;
the VL CDR1 comprises the amino acid sequence of SEQ ID NO:8; the VL CDR2 comprises the amino acid sequence of SEQ ID NO:15; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:79; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:29; the VH CDR2 comprises the amino acid sequence of SEQ ID NO:46; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:80; and the VH CDR3 comprises the amino acid sequence of SEQ ID NO:90;
the VL CDR1 comprises the amino acid sequence of SEQ ID NO:5; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:41; the VH CDR2 comprises the amino acid sequence of SEQ ID NO:60; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:32; the VH CDR2 comprises the amino acid sequence of SEQ ID NO:58; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:22; the VH CDR1 comprises the amino acid sequence of SEQ ID NO:34; the VH CDR2 comprises the amino acid sequence of SEQ ID NO:76; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:81; and the VH CDR3 comprises the amino acid sequence of SEQ ID NO:90;
the VL CDR1 comprises the amino acid sequence of SEQ ID NO:4; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:40; the VH CDR2 comprises the amino acid sequence of SEQ ID NO:60; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:28; the VH CDR2 comprises the amino acid sequence of SEQ ID NO:82; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:75; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:83; and the VH CDR3 comprises the amino acid sequence of SEQ ID NO:90;
the VL CDR1 comprises the amino acid sequence of SEQ ID NO:4; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:29; the VH CDR2 comprises the amino acid sequence of SEQ ID NO:84; and the VH CDR3 comprises the amino acid sequence of SEQ ID NO:90;
the VL CDR1 comprises the amino acid sequence of SEQ ID NO:10; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:25; the VH CDR2 comprises the amino acid sequence of SEQ ID NO:62; and the VH CDR3 comprises the amino acid sequence of SEQ ID NO:90;
the VL CDR1 comprises the amino acid sequence of SEQ ID NO:2; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:42; the VH CDR2 comprises the amino acid sequence of SEQ ID NO:76; and the VH CDR3 comprises the amino acid sequence of SEQ ID NO:90;
the VL CDR1 comprises the amino acid sequence of SEQ ID NO: 11; the VL CDR2 comprises the amino acid sequence of SEQ ID NO:17; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:50; and the VH CDR3 comprises the amino acid sequence of SEQ ID NO:90;
the VL CDR1 comprises the amino acid sequence of SEQ ID NO:5; the VL CDR2 comprises the amino acid sequence of SEQ ID NO:13; the VL CDR3 comprises the amino acid sequence of SEQ ID NO: 19; the VH CDR1 comprises the amino acid sequence of SEQ ID NO:29; the VH CDR2 comprises the amino acid sequence of SEQ ID NO:85; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:40; the VH CDR2 comprises the amino acid sequence of SEQ ID NO:46; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:27; the VH CDR2 comprises the amino acid sequence of SEQ ID NO:62; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:86; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:25; the VH CDR2 comprises the amino acid sequence of SEQ ID NO:62; and the VH CDR3 comprises the amino acid sequence of SEQ ID NO:90;
the VL CDR1 comprises the amino acid sequence of SEQ ID NO:10; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:62; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:58; and the VH CDR3 comprises the amino acid sequence of SEQ ID NO:90;
the VL CDR1 comprises the amino acid sequence of SEQ ID NO:12; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO43; the VH CDR2 comprises the amino acid sequence of SEQ ID NO:75; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:87; and the VH CDR3 comprises the amino acid sequence of SEQ ID NO:90; or
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:88; and the VH CDR3 comprises the amino acid sequence of SEQ ID NO:90.

In some embodiments, the VL is at least 80% identical to the amino acid sequence of any one of SEQ ID NOs:96-118; and the VH is at least 80% identical to the amino acid sequence of any one of SEQ ID NOs:119-190.

In some embodiments, the VL comprises the amino acid sequence of any one of SEQ ID NOs:96-118; and the VH comprises the amino acid sequence of any one of SEQ ID NOs:119-190.

In some embodiments, the VL comprises the amino acid sequence of SEQ ID NO:96 and the VH comprises the amino acid sequence of SEQ ID NO:119;
the VL comprises the amino acid sequence of SEQ ID NO:97 and the VH comprises the amino acid sequence of SEQ ID NO:120;
the VL comprises the amino acid sequence of SEQ ID NO:97 and the VH comprises the amino acid sequence of SEQ ID NO:121;
the VL comprises the amino acid sequence of SEQ ID NO:97 and the VH comprises the amino acid sequence of SEQ ID NO:122;
the VL comprises the amino acid sequence of SEQ ID NO:97 and the VH comprises the amino acid sequence of SEQ ID NO:123;
the VL comprises the amino acid sequence of SEQ ID NO:98 and the VH comprises the amino acid sequence of SEQ ID NO:124;
the VL comprises the amino acid sequence of SEQ ID NO:96 and the VH comprises the amino acid sequence of SEQ ID NO:125;
the VL comprises the amino acid sequence of SEQ ID NO:97 and the VH comprises the amino acid sequence of SEQ ID NO:126;
the VL comprises the amino acid sequence of SEQ ID NO:97 and the VH comprises the amino acid sequence of SEQ ID NO:127;
the VL comprises the amino acid sequence of SEQ ID NO:97 and the VH comprises the amino acid sequence of SEQ ID NO:128;
the VL comprises the amino acid sequence of SEQ ID NO:97 and the VH comprises the amino acid sequence of SEQ ID NO:125;
the VL comprises the amino acid sequence of SEQ ID NO:96 and the VH comprises the amino acid sequence of SEQ ID NO:129;
the VL comprises the amino acid sequence of SEQ ID NO:99 and the VH comprises the amino acid sequence of SEQ ID NO:130;
the VL comprises the amino acid sequence of SEQ ID NO: 100 and the VH comprises the amino acid sequence of SEQ ID NO:125;
the VL comprises the amino acid sequence of SEQ ID NO:101 and the VH comprises the amino acid sequence of SEQ ID NO:131;
the VL comprises the amino acid sequence of SEQ ID NO:102 and the VH comprises the amino acid sequence of SEQ ID NO:132;
the VL comprises the amino acid sequence of SEQ ID NO:103 and the VH comprises the amino acid sequence of SEQ ID NO:133;
the VL comprises the amino acid sequence of SEQ ID NO:104 and the VH comprises the amino acid sequence of SEQ ID NO:120;
the VL comprises the amino acid sequence of SEQ ID NO:97 and the VH comprises the amino acid sequence of SEQ ID NO:134;
the VL comprises the amino acid sequence of SEQ ID NO:97 and the VH comprises the amino acid sequence of SEQ ID NO:135;
the VL comprises the amino acid sequence of SEQ ID NO:97 and the VH comprises the amino acid sequence of SEQ ID NO:136;
the VL comprises the amino acid sequence of SEQ ID NO:105 and the VH comprises the amino acid sequence of SEQ ID NO:137;
the VL comprises the amino acid sequence of SEQ ID NO:97 and the VH comprises the amino acid sequence of SEQ ID NO:138;
the VL comprises the amino acid sequence of SEQ ID NO:97 and the VH comprises the amino acid sequence of SEQ ID NO:139;
the VL comprises the amino acid sequence of SEQ ID NO:97 and the VH comprises the amino acid sequence of SEQ ID NO:129;
the VL comprises the amino acid sequence of SEQ ID NO:106 and the VH comprises the amino acid sequence of SEQ ID NO:125;
the VL comprises the amino acid sequence of SEQ ID NO:96 and the VH comprises the amino acid sequence of SEQ ID NO:140;
the VL comprises the amino acid sequence of SEQ ID NO:107 and the VH comprises the amino acid sequence of SEQ ID NO:141;
the VL comprises the amino acid sequence of SEQ ID NO:108 and the VH comprises the amino acid sequence of SEQ ID NO:142;
the VL comprises the amino acid sequence of SEQ ID NO:97 and the VH comprises the amino acid sequence of SEQ ID NO:143;
the VL comprises the amino acid sequence of SEQ ID NO:97 and the VH comprises the amino acid sequence of SEQ ID NO:144;
the VL comprises the amino acid sequence of SEQ ID NO:97 and the VH comprises the amino acid sequence of SEQ ID NO:145;
the VL comprises the amino acid sequence of SEQ ID NO:97 and the VH comprises the amino acid sequence of SEQ ID NO:146;
the VL comprises the amino acid sequence of SEQ ID NO:97 and the VH comprises the amino acid sequence of SEQ ID NO:147;
the VL comprises the amino acid sequence of SEQ ID NO:97 and the VH comprises the amino acid sequence of SEQ ID NO:148;
the VL comprises the amino acid sequence of SEQ ID NO:96 and the VH comprises the amino acid sequence of SEQ ID NO:149;
the VL comprises the amino acid sequence of SEQ ID NO:101 and the VH comprises the amino acid sequence of SEQ ID NO:150;
the VL comprises the amino acid sequence of SEQ ID NO: 109 and the VH comprises the amino acid sequence of SEQ ID NO:151;
the VL comprises the amino acid sequence of SEQ ID NO:110 and the VH comprises the amino acid sequence of SEQ ID NO:137;
the VL comprises the amino acid sequence of SEQ ID NO:96 and the VH comprises the amino acid sequence of SEQ ID NO:152;
the VL comprises the amino acid sequence of SEQ ID NO:97 and the VH comprises the amino acid sequence of SEQ ID NO:153;
the VL comprises the amino acid sequence of SEQ ID NO:97 and the VH comprises the amino acid sequence of SEQ ID NO:154;
the VL comprises the amino acid sequence of SEQ ID NO:97 and the VH comprises the amino acid sequence of SEQ ID NO:155;
the VL comprises the amino acid sequence of SEQ ID NO:97 and the VH comprises the amino acid sequence of SEQ ID NO:156;
the VL comprises the amino acid sequence of SEQ ID NO:111 and the VH comprises the amino acid sequence of SEQ ID NO:157;
the VL comprises the amino acid sequence of SEQ ID NO:112 and the VH comprises the amino acid sequence of SEQ ID NO:158;
the VL comprises the amino acid sequence of SEQ ID NO:113 and the VH comprises the amino acid sequence of SEQ ID NO:159;
the VL comprises the amino acid sequence of SEQ ID NO:97 and the VH comprises the amino acid sequence of SEQ ID NO:160;
the VL comprises the amino acid sequence of SEQ ID NO:110 and the VH comprises the amino acid sequence of SEQ ID NO:140;
the VL comprises the amino acid sequence of SEQ ID NO:97 and the VH comprises the amino acid sequence of SEQ ID NO:161;
the VL comprises the amino acid sequence of SEQ ID NO:97 and the VH comprises the amino acid sequence of SEQ ID NO:162;
the VL comprises the amino acid sequence of SEQ ID NO:97 and the VH comprises the amino acid sequence of SEQ ID NO:163;
the VL comprises the amino acid sequence of SEQ ID NO:97 and the VH comprises the amino acid sequence of SEQ ID NO:164;
the VL comprises the amino acid sequence of SEQ ID NO:97 and the VH comprises the amino acid sequence of SEQ ID NO:165;
the VL comprises the amino acid sequence of SEQ ID NO:97 and the VH comprises the amino acid sequence of SEQ ID NO:166;
the VL comprises the amino acid sequence of SEQ ID NO:106 and the VH comprises the amino acid sequence of SEQ ID NO:167;
the VL comprises the amino acid sequence of SEQ ID NO:97 and the VH comprises the amino acid sequence of SEQ ID NO:168;
the VL comprises the amino acid sequence of SEQ ID NO:97 and the VH comprises the amino acid sequence of SEQ ID NO:169;
the VL comprises the amino acid sequence of SEQ ID NO: 102 and the VH comprises the amino acid sequence of SEQ ID NO:170;
the VL comprises the amino acid sequence of SEQ ID NO:97 and the VH comprises the amino acid sequence of SEQ ID NO:171;
the VL comprises the amino acid sequence of SEQ ID NO:114 and the VH comprises the amino acid sequence of SEQ ID NO:172;
the VL comprises the amino acid sequence of SEQ ID NO:97 and the VH comprises the amino acid sequence of SEQ ID NO:173;
the VL comprises the amino acid sequence of SEQ ID NO:97 and the VH comprises the amino acid sequence of SEQ ID NO:174;
the VL comprises the amino acid sequence of SEQ ID NO:97 and the VH comprises the amino acid sequence of SEQ ID NO:175;
the VL comprises the amino acid sequence of SEQ ID NO:101 and the VH comprises the amino acid sequence of SEQ ID NO:176;
the VL comprises the amino acid sequence of SEQ ID NO:101 and the VH comprises the amino acid sequence of SEQ ID NO:177;
the VL comprises the amino acid sequence of SEQ ID NO:115 and the VH comprises the amino acid sequence of SEQ ID NO:178;
the VL comprises the amino acid sequence of SEQ ID NO:98 and the VH comprises the amino acid sequence of SEQ ID NO:179;
the VL comprises the amino acid sequence of SEQ ID NO:116 and the VH comprises the amino acid sequence of SEQ ID NO:125;
the VL comprises the amino acid sequence of SEQ ID NO:102 and the VH comprises the amino acid sequence of SEQ ID NO:180;
the VL comprises the amino acid sequence of SEQ ID NO:97 and the VH comprises the amino acid sequence of SEQ ID NO:181;
the VL comprises the amino acid sequence of SEQ ID NO:97 and the VH comprises the amino acid sequence of SEQ ID NO:182;
the VL comprises the amino acid sequence of SEQ ID NO:97 and the VH comprises the amino acid sequence of SEQ ID NO:183;
the VL comprises the amino acid sequence of SEQ ID NO:97 and the VH comprises the amino acid sequence of SEQ ID NO:184;
the VL comprises the amino acid sequence of SEQ ID NO:97 and the VH comprises the amino acid sequence of SEQ ID NO:185;
the VL comprises the amino acid sequence of SEQ ID NO:97 and the VH comprises the amino acid sequence of SEQ ID NO:186;
the VL comprises the amino acid sequence of SEQ ID NO:117 and the VH comprises the amino acid sequence of SEQ ID NO:145;
the VL comprises the amino acid sequence of SEQ ID NO:97 and the VH comprises the amino acid sequence of SEQ ID NO:187;
the VL comprises the amino acid sequence of SEQ ID NO:118 and the VH comprises the amino acid sequence of SEQ ID NO:188;
the VL comprises the amino acid sequence of SEQ ID NO:97 and the VH comprises the amino acid sequence of SEQ ID NO:189;
the VL comprises the amino acid sequence of SEQ ID NO:97 and the VH comprises the amino acid sequence of SEQ ID NO:190; or the VL comprises the amino acid sequence of SEQ ID NO:97 and the VH comprises the amino acid sequence of SEQ ID NO:178.

In some embodiments, the antibody is a full-length antibody. In some embodiments, the antibody is a bispecific antibody, a single chain antibody (scFv), an Fab fragment, an F(ab′)2 fragment, an Fab′ fragment, an Fv fragment, an sc(Fv)₂, or a diabody.

In some embodiments, the antibody is conjugated to a toxic substance. In some embodiments, the toxic substance is a radioisotope or a cytotoxic agent. In some embodiments, the antibody is conjugated to a detectable label.

Aspects of the present disclosure provide a nucleic acid or a set of nucleic acids, which collectively encodes any one of the anti-B7-H4 antibodies described herein.

Aspects of the present disclosure provide an expression vector or a set of expression vectors comprising the nucleic acid or the set of nucleic acids encoding any one of the anti-B7-H4 antibodies described herein operably linked to a promoter.

Aspects of the present disclosure provide an isolated cell comprising the nucleic acid or the set of nucleic acids encoding any one of the anti-B7-H4 antibodies described herein or the expression vector or the set of expression vectors encoding any one of the anti-B7-H4 antibodies described herein.

Aspects of the present disclosure provide a method of making an antibody described herein comprising culturing an isolated cell comprising the nucleic acid or the set of nucleic acids encoding any one of the anti-B7-H4 antibodies described herein or the expression vector or the set of expression vectors encoding any one of the anti-B7-H4 antibodies described herein; and isolating the antibody.

Aspects of the present disclosure provide a pharmaceutical composition comprising an antibody, a nucleic acid or the set of nucleic acids encoding the antibody, an expression vector or the set of expression vectors encoding the antibody, or the isolated cell comprising the nucleic acid or the set of nucleic acids or the expression vector or the set of expression vectors, and a pharmaceutically acceptable carrier.

Aspects of the present disclosure provide a method of treating a cancer in a human subject in need thereof, the method comprising administering to the human subject an effective amount of an antibody or a pharmaceutical composition comprising the antibody.

In some embodiments, the cancer is ovarian cancer, breast cancer, endometrial cancer, bladder cancer, esophageal cancer, oral squamous cell carcinoma, brain cancer, prostate cancer, pancreatic cancer, cervical cancer, skin cancer, lung cancer, gastric cancer, or renal cell carcinoma.

In some embodiments, methods described herein further comprise administering to the human subject an additional therapy selected from the group consisting of a Janus tyrosine kinase (JAK) inhibitor, a phosphoinositide 3-kinase (PI3K) inhibitor, a standard of care therapy, or a combination thereof.

In some embodiments, the JAK inhibitor is ruxolitinib and itaticinib. In some embodiments, the PI3K inhibitor is parsaclisib. In some embodiments, the standard of care therapy is selected from the group consisting of a chemotherapy, a radiation therapy, a surgical therapy, an immunotherapy, or a combination thereof.

Aspects of the present disclosure provide a method for detecting human B7-H4, the method comprising contacting any one of the anti-B7-H4 antibodies described herein with a sample suspected of containing B7-H4, and detecting binding of the antibody to B7-H4.

In some embodiments, B7-H4 is expressed on a cell surface. In some embodiments, the contacting step is performed by administering the antibody to a subject.

Aspects of the present disclosure provide a kit comprising any of the anti-B7-H4 antibodies described herein, the nucleic acid or the set of nucleic acids encoding the antibody, the expression vector or the set of expression vectors encoding the antibody, or the isolated cell encoding the nucleic acid or the set of nucleic acids or the expression vector or the set of expression vectors, and instructions for use in treating a cancer in a human subject in need thereof, optionally with instructions for use in combination with an additional therapy.

### DETAILED DESCRIPTION

Provided herein are anti-B7-H4 antibodies and related nucleic acids, expression vectors, cells, and pharmaceutical compositions. The anti-B7-H4 antibodies described herein are useful in the treatment, prevention or diagnosis of cancer (e.g., ovarian cancer, breast cancer, endometrial cancer, bladder cancer, esophageal cancer, oral squamous cell carcinoma, brain cancer, prostate cancer, pancreatic cancer, cervical cancer, skin cancer, lung cancer, gastric cancer, and renal cell carcinoma).

### B7-H4 and Anti-B7-H4 Antibodies

B7-H4 (also known as B7x, B7S1, or VTCN1) is a transmembrane protein that binds to an unknown receptor on activated T cells resulting in inhibition of T-cell effector function via cell cycle arrest, decreased proliferation, and reduced IL-2 production. Various studies have demonstrated that B7-H4 plays a role in diminishing both CD4⁺ and CD8⁺ T-cell functionality. B7-H4 protein can be membrane bound or expressed in the cytoplasm.

The amino acid sequence of the mature human B7-H4 protein (amino acids 1-282 of GenBank Accession No. NP 078902.2) is;

(SEQ ID NO: 191)
MASLGQILFWSIISIIIILAGAIALIIGFGISGRHSITVTTVASAGNIG
EDGILSCTFEPDIKLSDIVIQWLKEGVLGLVHEFKEGKDELSEQDEMFR
GRTAVFADQVIVGNASLRLKNVQLTDAGTYKCYIITSKGKGNANLEYKT
GAFSMPEVNVDYNASSETLRCEAPRWFPQPTVVWASQVDQGANFSEVSN
TSFELNSENVTMKVVSVLYNVTINNTYSCMIENDIAKATGDIKVTESEI
KRRSHLQLLNSKASLCVSSFFAISWALLPLSPYLMLK

This disclosure provides anti-human B7-H4 antibodies that are useful in treating cancer.

In some embodiments, the anti-B7-H4 antibody is an anti-B7-H4 antibody that comprises one, two, three, four, five, and/or six CDRs of any one of the antibodies described herein. In some embodiments, an anti-B7-H4 antibody comprises (i) one, two, and/or three heavy chain CDRs of any one of the clones presented in Table 4, and/or (ii) one, two, and/or three light chain CDRs from any one of the clones presented in Table 4.

In some embodiments, an anti-B7-H4 antibody comprises (i) three heavy chain CDRs from any one of the clones presented in Table 4, and (ii) three light chain CDRs from any one of the clones presented in Table 4.

In some embodiments, an anti-B7-H4 antibody comprises a heavy chain CDR1, CDR2, and CDR3 and/or a light chain variable region CDR1, CDR2, and CDR3 from an antibody described herein. In some embodiments, an anti-B7-H4 antibody comprises a heavy chain CDR1. CDR2, and CDR3 and a light chain CDR1. CDR2, and CDR3 from an antibody described herein.

In some embodiments, an anti-B7-H4 antibody comprises a heavy chain CDR1, CDR2, and CDR3 and/or a light chain variable region CDR1, CDR2, and CDR3 from any clone disclosed herein. In some embodiments, an anti-B7-H4 antibody comprises a heavy chain CDR1, a heavy chain variable region CDR2, and a heavy chain variable region CDR3 from any clone disclosed herein. In other embodiments, an anti-B7-H4 antibody comprises a light chain variable region CDR1, a light chain variable region CDR2, and a light chain variable region CDR3 from any clone disclosed herein. In certain embodiments, an anti-B7-H4 antibody comprises a heavy chain CDR1, a heavy chain variable region CDR2, a heavy chain variable region CDR3, a light chain variable region CDR1, a light chain variable region CDR2, and a light chain variable region CDR3 from antibody any clone disclosed herein. In some embodiments, an anti-B7-H4 antibody is a variant of any clone disclosed herein.

In some embodiments, the anti-B7-H4 antibody is a variant of an anti-B7-H4 antibody described herein which comprises one to thirty conservative amino acid substitution(s), e.g., one to twenty-five, one to twenty, one to fifteen, one to ten, one to five, or one to three conservative amino acid substitution(s). In some embodiments, the conservative amino acid substitution(s) is in a CDR of the antibody. In some embodiments, the conservative amino acid substitution(s) is not in a CDR of the antibody. In some embodiments, the conservative amino acid substitution(s) is in a framework region of the antibody.

In some embodiments, the anti-B7-H4 antibody comprises: (a) a heavy chain variable region CDR1 comprising GFTFSSYS (SEQ ID NO:23), a heavy chain variable region CDR2 comprising ISGSGGST (SEQ ID NO:44), and a heavy chain variable region CDR3 comprising ARKRWYGMDV (SEQ ID NO:90); and/or (b) a light chain variable region CDR1 comprising NIGSKS (SEQ ID NO:1); a light chain variable region CDR2 comprising DDSDRPS (SEQ ID NO:13), and a light chain variable region CDR3 comprising QVWDSRTYV (SEQ ID NO:18). In some embodiments, an anti-B7-H4 antibody comprises a heavy chain variable region CDR1 comprising GFTFSSYS (SEQ ID NO:23), a heavy chain variable region CDR2 comprising ISGSGGST (SEQ ID NO:44), and a heavy chain variable region CDR3 comprising ARKRWYGMDV (SEQ ID NO:90). In some embodiments, an anti-B7-H4 antibody comprises a light chain variable region CDR1 comprising NIGSKS (SEQ ID NO:1), a light chain variable region CDR2 comprising DDSDRPS (SEQ ID NO:13), and a light chain variable region CDR3 comprising QVWDSRTYV (SEQ ID NO:18). In some embodiments, an anti-B7-H4 antibody comprises (a) a heavy chain variable region CDR1 comprising GFTFSSYS (SEQ ID NO:23), a heavy chain variable region CDR2 comprising ISGSGGST (SEQ ID NO:44), and a heavy chain variable region CDR3 comprising ARKRWYGMDV (SEQ ID NO:90), and (b) a light chain variable region CDR1 comprising NIGSKS (SEQ ID NO:1), a light chain variable region CDR2 comprising DDSDRPS (SEQ ID NO:13), and a light chain variable region CDR3 comprising QVWDSRTYV (SEQ ID NO:18). In some embodiments, an anti-B7-H4 antibody comprises (a) a heavy chain variable region comprising a heavy chain CDR1 comprising GFTFSSYS (SEQ ID NO:23), a heavy chain CDR2 comprising ISGSGGST (SEQ ID NO:44), and a heavy chain CDR3 comprising ARKRWYGMDV (SEQ ID NO:90), or (b) a light chain variable region comprising light chain CDR1 comprising NIGSKS (SEQ ID NO:1), a light chain CDR2 comprising DDSDRPS (SEQ ID NO:13), and a light chain CDR3 comprising QVWDSRTYV (SEQ ID NO:18).

In some embodiments, an anti-B7-H4 antibody comprises: a heavy chain variable region CDR1 comprising GFTFSSYS (SEQ ID NO:23), or a variant thereof comprising 1, 2, 3, or 4 amino acid substitutions; a heavy chain variable region CDR2 comprising ISGSGGST (SEQ ID NO:44), or a variant thereof comprising 1, 2, 3, or 4 amino acid substitutions; a heavy chain variable region CDR3 comprising ARKRWYGMDV (SEQ ID NO: 90), or a variant thereof comprising 1, 2, 3, or 4 amino acid substitutions: a light chain variable region CDR1 comprising NIGSKS (SEQ ID NO:1), or a variant thereof comprising 1, 2, 3, or 4 amino acid substitutions; a light chain variable region CDR2 comprising DDSDRPS (SEQ ID NO:13), or a variant thereof comprising 1, 2, 3, or 4 amino acid substitutions; and a light chain variable region CDR3 comprising QVWDSRTYV (SEQ ID NO:18) or a variant thereof comprising 1, 2, 3, or 4 amino acid substitutions. In some embodiments, a CDR comprises one amino acid substitution. In some embodiments, a CDR comprises two amino acid substitutions. In some embodiments, a CDR comprises three amino acid substitutions. In some embodiments, a CDR comprises four amino acid substitutions. In some embodiments, the one or more amino acid substitutions are conservative substitutions. In some embodiments, the CDR is a heavy chain CDR1. In some embodiments, the CDR is a heavy chain variable region CDR2. In some embodiments, the CDR is a heavy chain variable region CDR3. In some embodiments, the CDR is a light chain variable region CDR1. In some embodiments, the CDR is a light chain variable region CDR2. In some embodiments, the CDR is a light chain variable region CDR3. In some embodiments, the one or more substitutions are made as part of an affinity maturation process. In some embodiments, the one or more substitutions are made as part of an optimization process.

In some embodiments, an anti-B7-H4 antibody comprises a heavy chain variable region CDR1, a heavy chain variable region CDR2, and a heavy chain variable region CDR3, each of which correspond to the heavy chain variable region CDRs set forth in Table 4 for a single clone, and a light chain variable region CDR1, a light chain variable region VL CDR2, and a light chain variable region VL CDR3, each of which correspond to the VL CDRs set forth in Table 4 for a single clone.

In some embodiments, an anti-B7-H4 antibody comprises a heavy chain variable region CDR1, a heavy chain variable region CDR2, a heavy chain variable region CDR3, a light chain variable region CDR1, a light chain variable region CDR2, and a light chain variable region CDR3, each of which correspond to the VH and VL CDRs set forth in Table 4 for a single clone.

In some embodiments, the anti-B7-H4 antibody of the disclosure comprises a light chain variable region (VL) comprising VL CDR1, VL CDR2, and VL CDR3, wherein: the VL CDR1 comprises the amino acid sequence X₁X₂X₃X₄KX₅ (SEQ ID NO:91), wherein X₁ is N, D, or K, wherein X₂ is 1 or L, wherein X₃ is G, V, or W, wherein X₄ is S, K, D, F, L, M, V, Y, T, or G, and wherein X₅ is S, W, M, or A, the VL CDR2 comprises the amino acid sequence X₆DX₇DRX₈X₉(SEQ ID NO:92), wherein X₆ is D or E, wherein X₇ is S, I, or V, wherein X₅ is P or S, wherein X₉ is S, A, or P, and the VL CDR3 comprises the amino acid sequence QVWX₁₀X₁₁X₁₂X₁₃X₁₄X₁₅X₁₆X₁₇ (SEQ ID NO:93), wherein X₁₀ is A, D or G, wherein X₁₁ is S or R, wherein X₂ is T. G. R, or Y, wherein X₁₃ is T, M, S, or Q, wherein X₁₄ is A, I, V, Y, or M, wherein X₁₅ is P. V, or I, wherein X₁₆ is S or absent, and wherein X₁₇ is V or absent; and a heavy chain variable region (VH) comprising VH CDR1, VH CDR2, and VH CDR3, wherein: the VH CDR1 comprises the amino acid sequence X₁₈X₁₉X₂₀X₂₁X₂₂X₂₃X₂₄X₂₅ (SEQ ID NO:94), wherein X₁₅ is A or G, wherein X₁₉ is F or L, wherein X₂₀ is T, I, or S, wherein X₂₁ is F or Y, wherein X₂₂ is S, D, G, or T, wherein X₂₁ is R, T, S, N, D, or I, wherein X₂₄ is F, K, R, Q, or Y, and wherein X₂₅ is A or S; the VH CDR2 comprises the amino acid sequence X₂₆X₂₇X₂₈X₂₉X₃₀X₃₁X₃₂X₃₃ (SEQ ID NO:95), wherein X₂₆ is I or V, X₂₇ is D or S, wherein X₂₁ is A, G. S or Y, wherein X₂₉ is T, S, P. R or E; wherein X₃₀ is G or A, wherein X₃₁ is R, A, D, T, M, F, G, K, L, S, or Y, wherein X₃₂ is T, S. V, G, or N, and wherein X₃₃ is I, T, R, A, S, K, M, V, or Q; and the VH CDR3 comprises the amino acid sequence ARKRWYGMDV (SEQ ID NO:90).

TABLE 1
Consensus CDR sequences.

| CDR | SEQ ID NO: | Sequence |
|---|---|---|
| VL CDR1 | 91 | X₁X₂X₃X₄KX₅ |
| | | wherein X₁ is N, D, or K; wherein X₂ is I or L; wherein |
| | | X₃ is G, V, or W; wherein X₄ is S, K, D, F, L, M, V, Y, |
| | | T, or G; and wherein X₅ is S, W, M, or A. |
| VL CDR2 | 92 | X₆DX₇DRX₈X₉ |
| | | wherein X₆ is D or E; wherein X₇ is S, I, or V; wherein |
| | | X₈ is P or S, wherein X₉ is S, A, or P. |
| VL CDR3 | 93 | QVWX₁₀X₁₁X₁₂X₁₃X₁₄X₁₅X₁₆X₁₇ |
| | | wherein X₁₀ is A, D or G; wherein X₁₁ is S or R; |
| | | wherein X₁₂ is T, G, R, or Y; wherein X₁₃ is T, M, S, or |
| | | Q; wherein X₁₄ is A, I, V, Y, or M, wherein X₁₅ is P, V, |
| | | or I; wherein X₁₆ is S or absent; and wherein X₁₇ is V or |
| | | absent. |
| VH CDR1 | 94 | X₁₈X₁₉X₂₀X₂₁X₂₂X₂₃X₂₄X₂₅ |
| | | wherein X₁₈ is A or G; wherein X₁₉ is F or L; wherein |
| | | X₂₀ is T, I, or S; wherein X₂₁ is For Y; wherein X₂₂ is S, |
| | | D, G, or T; wherein X₂₃ is R, T, S, N, D, or I; wherein |
| | | X₂₄ is F, K, R, Q, or Y; and wherein X₂₅ is A or S. |
| VH CDR2 | 95 | X₂₆X₂₇X₂₈X₂₉X₃₀X₃₁X₃₂X₃₃ |
| | | wherein X₂₆ is I or V; X₂₇ is D or S; wherein X₂₈ is A, |
| | | G, S or Y; wherein X₂₉ is T, S, P, R or E; wherein X₃₀ is |
| | | G or A; wherein X₃₁ is R, A, D, T, M, F, G, K, L, S, or |
| | | Y; wherein X₃₂ is T, S, V, G, or N; and wherein X₃₃ is I, |
| | | T, R, A, S, K, M, V, or Q. |

In some embodiments, an anti-B7-H4 antibody comprises a heavy chain variable region comprising an amino acid sequence that has the three VH CDRs of any anti-B7-H4 clone disclosed herein and which has at least about 75%, 80%, 85%, 90%, 95%, 96%, 97%, 98%, 99%, or 100% identity to the sequence set forth in SEQ ID NO:119. In some embodiments, an anti-B7-H4 antibody comprises a light chain variable region comprising an amino acid sequence that has the three VL CDRs of any anti-B7-H4 clone disclosed herein and which has at least about 75%, 80%, 85%, 90%, 95%, 96%, 97%, 98%, 99%, or 100% identity to the sequence set forth in SEQ ID NO:96.

In some embodiments, an anti-B7-H4 antibody comprises a heavy chain variable region comprising an amino acid sequence that has the three VH CDRs of any anti-B7-H4 clone disclosed herein and which has at least about 75%, 80%, 85%, 90%, 95%, 96%, 97%, 98%, 99%, or 100% identity to any one of the VH sequences set forth in Table 5 and a light chain variable region comprising an amino acid sequence that has the three VL CDRs of any anti-B7-H4 clone disclosed herein and which has at least about 75%, 80%, 85%, 90%, 95%, 96%, 97%, 98%, 99%, or 100% identity to any one of the VL sequences set forth in Table 5.

In some embodiments, an anti-B7-H4 antibody comprises a heavy chain variable region having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity sequence identity to SEQ ID NO:119 and/or a light chain variable region having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity sequence identity to SEQ ID NO:96. In some embodiments, an anti-B7-H4 antibody comprises a heavy chain variable region having at least 80%, at least 85%, at least 86%, at least 87%, at least 88%, at least 89%, at least 900%, at least 91%, at least 92%, at least 93%, at least 94%, at least 95%, at least 96%, at least 97%, at least 98%, at least 99%, or 100% identity sequence identity to SEQ ID NO:119. In some embodiments, an anti-B7-H4 antibody comprises a light chain variable region having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity sequence identity to SEQ ID NO: 96.

In some embodiments, an anti-B7-H4 antibody comprises a heavy chain variable region with one or more (e.g., 1, 2, or 3) substitutions, deletions, or insertions in the sequence set forth in SEQ ID NO:119. In some embodiment, an anti-B7-H4 antibody comprises a light chain variable region with one or more (e.g., 1, 2, or 3) substitutions, deletions, or insertions in the sequence set forth in SEQ ID NO:96. In some embodiments, an anti-B7-H4 antibody comprises a heavy chain variable region with one or more (e.g., 1, 2, or 3) substitutions, deletions, or insertions in the sequence set forth in SEQ ID NO:119 and a light chain variable region with one or more substitutions, deletions, or insertions in the sequence set forth in SEQ ID NO:96.

In some embodiments, an anti-B7-H4 antibody comprises a heavy chain variable region comprising any one of the VH sequences set forth in Table 5. In some embodiments, an anti-B7-H4 antibody comprises a light chain variable region comprising any one of the VL sequences set forth in Table 5. In some embodiments, an anti-B7-H4 antibody comprises a heavy chain variable region comprising any one of the VH sequences set forth in Table 5 and a light chain variable region comprising any one of the VL sequences set forth in Table 5.

In some embodiments, an anti-B7-H4 antibody comprises a heavy chain variable region comprising SEQ ID NO:119. In some embodiments, an anti-B7-H4 antibody comprises a light chain variable region comprising SEQ ID NO: 96. In some embodiments, an anti-B7-H4 antibody comprises a heavy chain variable region comprising SEQ ID NO:119 and a light chain variable region comprising SEQ ID NO: 96.

In some embodiments, an anti-B7-H4 antibody comprises a heavy chain variable region comprising SEQ ID NO:120. In some embodiments, an anti-B7-H4 antibody comprises a light chain variable region comprising SEQ ID NO:97. In some embodiments, an anti-B7-H4 antibody comprises a heavy chain variable region comprising SEQ ID NO:120 and a light chain variable region comprising SEQ ID NO:97.

In some embodiments, an anti-B7-H4 antibody comprises a heavy chain variable region comprising SEQ ID NO:121. In some embodiments, an anti-B7-H4 antibody comprises a light chain variable region comprising SEQ ID NO:97. In some embodiments, an anti-B7-H4 antibody comprises a heavy chain variable region comprising SEQ ID NO:121 and a light chain variable region comprising SEQ ID NO:97.

In some embodiments, an anti-B7-H4 antibody comprises a heavy chain variable region comprising SEQ ID NO:122. In some embodiments, an anti-B7-H4 antibody comprises a light chain variable region comprising SEQ ID NO:97. In some embodiments, an anti-B7-H4 antibody comprises a heavy chain variable region comprising SEQ ID NO:122 and a light chain variable region comprising SEQ ID NO:97.

In some embodiments, an anti-B7-H4 antibody comprises a heavy chain variable region comprising SEQ ID NO:123. In some embodiments, an anti-B7-H4 antibody comprises a light chain variable region comprising SEQ ID NO:97. In some embodiments, an anti-B7-H4 antibody comprises a heavy chain variable region comprising SEQ ID NO:123 and a light chain variable region comprising SEQ ID NO:97.

In some embodiments, an anti-B7-H4 antibody comprises a heavy chain variable region comprising SEQ ID NO:124. In some embodiments, an anti-B7-H4 antibody comprises a light chain variable region comprising SEQ ID NO:98. In some embodiments, an anti-B7-H4 antibody comprises a heavy chain variable region comprising SEQ ID NO:124 and a light chain variable region comprising SEQ ID NO:98.

In some embodiments, an anti-B7-H4 antibody comprises a heavy chain variable region comprising SEQ ID NO:125. In some embodiments, an anti-B7-H4 antibody comprises a light chain variable region comprising SEQ ID NO:96. In some embodiments, an anti-B7-H4 antibody comprises a heavy chain variable region comprising SEQ ID NO:125 and a light chain variable region comprising SEQ ID NO:96.

In some embodiments, an anti-B7-H4 antibody comprises a heavy chain variable region comprising SEQ ID NO:126. In some embodiments, an anti-B7-H4 antibody comprises a light chain variable region comprising SEQ ID NO:97. In some embodiments, an anti-B7-H4 antibody comprises a heavy chain variable region comprising SEQ ID NO:126 and a light chain variable region comprising SEQ ID NO:97.

In some embodiments, an anti-B7-H4 antibody comprises a heavy chain variable region comprising SEQ ID NO:127. In some embodiments, an anti-B7-H4 antibody comprises a light chain variable region comprising SEQ ID NO:97. In some embodiments, an anti-B7-H4 antibody comprises a heavy chain variable region comprising SEQ ID NO:127 and a light chain variable region comprising SEQ ID NO:97.

In some embodiments, an anti-B7-H4 antibody comprises a heavy chain variable region comprising SEQ ID NO:128. In some embodiments, an anti-B7-H4 antibody comprises a light chain variable region comprising SEQ ID NO:97. In some embodiments, an anti-B7-H4 antibody comprises a heavy chain variable region comprising SEQ ID NO:128 and a light chain variable region comprising SEQ ID NO:97.

In some embodiments, an anti-B7-H4 antibody comprises a heavy chain variable region comprising SEQ ID NO:125. In some embodiments, an anti-B7-H4 antibody comprises a light chain variable region comprising SEQ ID NO:97. In some embodiments, an anti-B7-H4 antibody comprises a heavy chain variable region comprising SEQ ID NO:125 and a light chain variable region comprising SEQ ID NO:97.

In some embodiments, an anti-B7-H4 antibody comprises a heavy chain variable region comprising SEQ ID NO:129. In some embodiments, an anti-B7-H4 antibody comprises a light chain variable region comprising SEQ ID NO:96. In some embodiments, an anti-B7-H4 antibody comprises a heavy chain variable region comprising SEQ ID NO:129 and a light chain variable region comprising SEQ ID NO:96.

In some embodiments, an anti-B7-H4 antibody comprises a heavy chain variable region comprising SEQ ID NO:130. In some embodiments, an anti-B7-H4 antibody comprises a light chain variable region comprising SEQ ID NO:99. In some embodiments, an anti-B7-H4 antibody comprises a heavy chain variable region comprising SEQ ID NO:130 and a light chain variable region comprising SEQ ID NO:99.

In some embodiments, an anti-B7-H4 antibody comprises a heavy chain variable region comprising SEQ ID NO: 125. In some embodiments, an anti-B7-H4 antibody comprises a light chain variable region comprising SEQ ID NO:100. In some embodiments, an anti-B7-H4 antibody comprises a heavy chain variable region comprising SEQ ID NO:125 and a light chain variable region comprising SEQ ID NO:100.

In some embodiments, an anti-B7-H4 antibody comprises a heavy chain variable region comprising SEQ ID NO:131. In some embodiments, an anti-B7-H4 antibody comprises a light chain variable region comprising SEQ ID NO: 101. In some embodiments, an anti-B7-H4 antibody comprises a heavy chain variable region comprising SEQ ID NO:131 and a light chain variable region comprising SEQ ID NO:101.

In some embodiments, an anti-B7-H4 antibody comprises a heavy chain variable region comprising SEQ ID NO:132. In some embodiments, an anti-B7-H4 antibody comprises a light chain variable region comprising SEQ ID NO:102. In some embodiments, an anti-B7-H4 antibody comprises a heavy chain variable region comprising SEQ ID NO:132 and a light chain variable region comprising SEQ ID NO:102.

In some embodiments, an anti-B7-H4 antibody comprises a heavy chain variable region comprising SEQ ID NO:133. In some embodiments, an anti-B7-H4 antibody comprises a light chain variable region comprising SEQ ID NO:103. In some embodiments, an anti-B7-H4 antibody comprises a heavy chain variable region comprising SEQ ID NO:133 and a light chain variable region comprising SEQ ID NO:103.

In some embodiments, an anti-B7-H4 antibody comprises a heavy chain variable region comprising SEQ ID NO:120. In some embodiments, an anti-B7-H4 antibody comprises a light chain variable region comprising SEQ ID NO:104. In some embodiments, an anti-B7-H4 antibody comprises a heavy chain variable region comprising SEQ ID NO:120 and a light chain variable region comprising SEQ ID NO:104.

In some embodiments, an anti-B7-H4 antibody comprises a heavy chain variable region comprising SEQ ID NO:134. In some embodiments, an anti-B7-H4 antibody comprises a light chain variable region comprising SEQ ID NO:97. In some embodiments, an anti-B7-H4 antibody comprises a heavy chain variable region comprising SEQ ID NO:134 and a light chain variable region comprising SEQ ID NO:97.

In some embodiments, an anti-B7-H4 antibody comprises a heavy chain variable region comprising SEQ ID NO:135. In some embodiments, an anti-B7-H4 antibody comprises a light chain variable region comprising SEQ ID NO:97. In some embodiments, an anti-B7-H4 antibody comprises a heavy chain variable region comprising SEQ ID NO: 135 and a light chain variable region comprising SEQ ID NO:97.

In some embodiments, an anti-B7-H4 antibody comprises a heavy chain variable region comprising SEQ ID NO:136. In some embodiments, an anti-B7-H4 antibody comprises a light chain variable region comprising SEQ ID NO:97. In some embodiments, an anti-B7-H4 antibody comprises a heavy chain variable region comprising SEQ ID NO:136 and a light chain variable region comprising SEQ ID NO:97.

In some embodiments, an anti-B7-H4 antibody comprises a heavy chain variable region comprising SEQ ID NO:137. In some embodiments, an anti-B7-H4 antibody comprises a light chain variable region comprising SEQ ID NO:105. In some embodiments, an anti-B7-H4 antibody comprises a heavy chain variable region comprising SEQ ID NO:137 and a light chain variable region comprising SEQ ID NO:105.

In some embodiments, an anti-B7-H4 antibody comprises a heavy chain variable region comprising SEQ ID NO:138. In some embodiments, an anti-B7-H4 antibody comprises a light chain variable region comprising SEQ ID NO:97. In some embodiments, an anti-B7-H4 antibody comprises a heavy chain variable region comprising SEQ ID NO:138 and a light chain variable region comprising SEQ ID NO:97.

In some embodiments, an anti-B7-H4 antibody comprises a heavy chain variable region comprising SEQ ID NO:139. In some embodiments, an anti-B7-H4 antibody comprises a light chain variable region comprising SEQ ID NO:97. In some embodiments, an anti-B7-H4 antibody comprises a heavy chain variable region comprising SEQ ID NO:139 and a light chain variable region comprising SEQ ID NO:97.

In some embodiments, an anti-B7-H4 antibody comprises a heavy chain variable region comprising SEQ ID NO:129. In some embodiments, an anti-B7-H4 antibody comprises a light chain variable region comprising SEQ ID NO:97. In some embodiments, an anti-B7-H4 antibody comprises a heavy chain variable region comprising SEQ ID NO:129 and a light chain variable region comprising SEQ ID NO:97.

In some embodiments, an anti-B7-H4 antibody comprises a heavy chain variable region comprising SEQ ID NO:125. In some embodiments, an anti-B7-H4 antibody comprises a light chain variable region comprising SEQ ID NO:106. In some embodiments, an anti-B7-H4 antibody comprises a heavy chain variable region comprising SEQ ID NO:125 and a light chain variable region comprising SEQ ID NO:106.

In some embodiments, an anti-B7-H4 antibody comprises a heavy chain variable region comprising SEQ ID NO:140. In some embodiments, an anti-B7-H4 antibody comprises a light chain variable region comprising SEQ ID NO:96. In some embodiments, an anti-B7-H4 antibody comprises a heavy chain variable region comprising SEQ ID NO:140 and a light chain variable region comprising SEQ ID NO:96.

In some embodiments, an anti-B7-H4 antibody comprises a heavy chain variable region comprising SEQ ID NO:141. In some embodiments, an anti-B7-H4 antibody comprises a light chain variable region comprising SEQ ID NO:107. In some embodiments, an anti-B7-H4 antibody comprises a heavy chain variable region comprising SEQ ID NO:141 and a light chain variable region comprising SEQ ID NO:107.

In some embodiments, an anti-B7-H4 antibody comprises a heavy chain variable region comprising SEQ ID NO:142. In some embodiments, an anti-B7-H4 antibody comprises a light chain variable region comprising SEQ ID NO:108. In some embodiments, an anti-B7-H4 antibody comprises a heavy chain variable region comprising SEQ ID NO:142 and a light chain variable region comprising SEQ ID NO:108.

In some embodiments, an anti-B7-H4 antibody comprises a heavy chain variable region comprising SEQ ID NO:143. In some embodiments, an anti-B7-H4 antibody comprises a light chain variable region comprising SEQ ID NO:97. In some embodiments, an anti-B7-H4 antibody comprises a heavy chain variable region comprising SEQ ID NO:143 and a light chain variable region comprising SEQ ID NO:97.

In some embodiments, an anti-B7-H4 antibody comprises a heavy chain variable region comprising SEQ ID NO:144. In some embodiments, an anti-B7-H4 antibody comprises a light chain variable region comprising SEQ ID NO:97. In some embodiments, an anti-B7-H4 antibody comprises a heavy chain variable region comprising SEQ ID NO:144 and a light chain variable region comprising SEQ ID NO:97.

In some embodiments, an anti-B7-H4 antibody comprises a heavy chain variable region comprising SEQ ID NO:145. In some embodiments, an anti-B7-H4 antibody comprises a light chain variable region comprising SEQ ID NO:97. In some embodiments, an anti-B7-H4 antibody comprises a heavy chain variable region comprising SEQ ID NO:145 and a light chain variable region comprising SEQ ID NO:97.

In some embodiments, an anti-B7-H4 antibody comprises a heavy chain variable region comprising SEQ ID NO: 146. In some embodiments, an anti-B7-H4 antibody comprises a light chain variable region comprising SEQ ID NO:97. In some embodiments, an anti-B7-H4 antibody comprises a heavy chain variable region comprising SEQ ID NO:146 and a light chain variable region comprising SEQ ID NO:97.

In some embodiments, an anti-B7-H4 antibody comprises a heavy chain variable region comprising SEQ ID NO: 147. In some embodiments, an anti-B7-H4 antibody comprises a light chain variable region comprising SEQ ID NO:97. In some embodiments, an anti-B7-H4 antibody comprises a heavy chain variable region comprising SEQ ID NO:147 and a light chain variable region comprising SEQ ID NO:97.

In some embodiments, an anti-B7-H4 antibody comprises a heavy chain variable region comprising SEQ ID NO:148. In some embodiments, an anti-B7-H4 antibody comprises a light chain variable region comprising SEQ ID NO:97. In some embodiments, an anti-B7-H4 antibody comprises a heavy chain variable region comprising SEQ ID NO:148 and a light chain variable region comprising SEQ ID NO:97.

In some embodiments, an anti-B7-H4 antibody comprises a heavy chain variable region comprising SEQ ID NO:149. In some embodiments, an anti-B7-H4 antibody comprises a light chain variable region comprising SEQ ID NO:96. In some embodiments, an anti-B7-H4 antibody comprises a heavy chain variable region comprising SEQ ID NO:149 and a light chain variable region comprising SEQ ID NO:96.

In some embodiments, an anti-B7-H4 antibody comprises a heavy chain variable region comprising SEQ ID NO:150. In some embodiments, an anti-B7-H4 antibody comprises a light chain variable region comprising SEQ ID NO:101. In some embodiments, an anti-B7-H4 antibody comprises a heavy chain variable region comprising SEQ ID NO:150 and a light chain variable region comprising SEQ ID NO:101.

In some embodiments, an anti-B7-H4 antibody comprises a heavy chain variable region comprising SEQ ID NO:151. In some embodiments, an anti-B7-H4 antibody comprises a light chain variable region comprising SEQ ID NO:109. In some embodiments, an anti-B7-H4 antibody comprises a heavy chain variable region comprising SEQ ID NO:151 and a light chain variable region comprising SEQ ID NO:109.

In some embodiments, an anti-B7-H4 antibody comprises a heavy chain variable region comprising SEQ ID NO:137. In some embodiments, an anti-B7-H4 antibody comprises a light chain variable region comprising SEQ ID NO:110. In some embodiments, an anti-B7-H4 antibody comprises a heavy chain variable region comprising SEQ ID NO:137 and a light chain variable region comprising SEQ ID NO:110.

In some embodiments, an anti-B7-H4 antibody comprises a heavy chain variable region comprising SEQ ID NO:152. In some embodiments, an anti-B7-H4 antibody comprises a light chain variable region comprising SEQ ID NO:96. In some embodiments, an anti-B7-H4 antibody comprises a heavy chain variable region comprising SEQ ID NO: 152 and a light chain variable region comprising SEQ ID NO:96.

In some embodiments, an anti-B7-H4 antibody comprises a heavy chain variable region comprising SEQ ID NO:153. In some embodiments, an anti-B7-H4 antibody comprises a light chain variable region comprising SEQ ID NO:97. In some embodiments, an anti-B7-H4 antibody comprises a heavy chain variable region comprising SEQ ID NO:153 and a light chain variable region comprising SEQ ID NO:97.

In some embodiments, an anti-B7-H4 antibody comprises a heavy chain variable region comprising SEQ ID NO:154. In some embodiments, an anti-B7-H4 antibody comprises a light chain variable region comprising SEQ ID NO:97. In some embodiments, an anti-B7-H4 antibody comprises a heavy chain variable region comprising SEQ ID NO:154 and a light chain variable region comprising SEQ ID NO:97.

In some embodiments, an anti-B7-H4 antibody comprises a heavy chain variable region comprising SEQ ID NO:155. In some embodiments, an anti-B7-H4 antibody comprises a light chain variable region comprising SEQ ID NO:97. In some embodiments, an anti-B7-H4 antibody comprises a heavy chain variable region comprising SEQ ID NO:155 and a light chain variable region comprising SEQ ID NO:97.

In some embodiments, an anti-B7-H4 antibody comprises a heavy chain variable region comprising SEQ ID NO:156. In some embodiments, an anti-B7-H4 antibody comprises a light chain variable region comprising SEQ ID NO:97. In some embodiments, an anti-B7-H4 antibody comprises a heavy chain variable region comprising SEQ ID NO:156 and a light chain variable region comprising SEQ ID NO:97.

In some embodiments, an anti-B7-H4 antibody comprises a heavy chain variable region comprising SEQ ID NO:157. In some embodiments, an anti-B7-H4 antibody comprises a light chain variable region comprising SEQ ID NO:111. In some embodiments, an anti-B7-H4 antibody comprises a heavy chain variable region comprising SEQ ID NO:157 and a light chain variable region comprising SEQ ID NO:111.

In some embodiments, an anti-B7-H4 antibody comprises a heavy chain variable region comprising SEQ ID NO:158. In some embodiments, an anti-B7-H4 antibody comprises a light chain variable region comprising SEQ ID NO:112. In some embodiments, an anti-B7-H4 antibody comprises a heavy chain variable region comprising SEQ ID NO:158 and a light chain variable region comprising SEQ ID NO:112.

In some embodiments, an anti-B7-H4 antibody comprises a heavy chain variable region comprising SEQ ID NO:159. In some embodiments, an anti-B7-H4 antibody comprises a light chain variable region comprising SEQ ID NO: 113. In some embodiments, an anti-B7-H4 antibody comprises a heavy chain variable region comprising SEQ ID NO:159 and a light chain variable region comprising SEQ ID NO:113.

In some embodiments, an anti-B7-H4 antibody comprises a heavy chain variable region comprising SEQ ID NO:160. In some embodiments, an anti-B7-H4 antibody comprises a light chain variable region comprising SEQ ID NO:97. In some embodiments, an anti-B7-H4 antibody comprises a heavy chain variable region comprising SEQ ID NO:160 and a light chain variable region comprising SEQ ID NO:97.

In some embodiments, an anti-B7-H4 antibody comprises a heavy chain variable region comprising SEQ ID NO:140. In some embodiments, an anti-B7-H4 antibody comprises a light chain variable region comprising SEQ ID NO:110. In some embodiments, an anti-B7-H4 antibody comprises a heavy chain variable region comprising SEQ ID NO:140 and a light chain variable region comprising SEQ ID NO:110.

In some embodiments, an anti-B7-H4 antibody comprises a heavy chain variable region comprising SEQ ID NO:161. In some embodiments, an anti-B7-H4 antibody comprises a light chain variable region comprising SEQ ID NO:97. In some embodiments, an anti-B7-H4 antibody comprises a heavy chain variable region comprising SEQ ID NO:161 and a light chain variable region comprising SEQ ID NO:97.

In some embodiments, an anti-B7-H4 antibody comprises a heavy chain variable region comprising SEQ ID NO:162. In some embodiments, an anti-B7-H4 antibody comprises a light chain variable region comprising SEQ ID NO:97. In some embodiments, an anti-B7-H4 antibody comprises a heavy chain variable region comprising SEQ ID NO:162 and a light chain variable region comprising SEQ ID NO:97.

In some embodiments, an anti-B7-H4 antibody comprises a heavy chain variable region comprising SEQ ID NO:163. In some embodiments, an anti-B7-H4 antibody comprises a light chain variable region comprising SEQ ID NO:97. In some embodiments, an anti-B7-H4 antibody comprises a heavy chain variable region comprising SEQ ID NO:163 and a light chain variable region comprising SEQ ID NO:97.

In some embodiments, an anti-B7-H4 antibody comprises a heavy chain variable region comprising SEQ ID NO: 164. In some embodiments, an anti-B7-H4 antibody comprises a light chain variable region comprising SEQ ID NO:97. In some embodiments, an anti-B7-H4 antibody comprises a heavy chain variable region comprising SEQ ID NO:164 and a light chain variable region comprising SEQ ID NO:97.

In some embodiments, an anti-B7-H4 antibody comprises a heavy chain variable region comprising SEQ ID NO: 165. In some embodiments, an anti-B7-H4 antibody comprises a light chain variable region comprising SEQ ID NO:97. In some embodiments, an anti-B7-H4 antibody comprises a heavy chain variable region comprising SEQ ID NO:165 and a light chain variable region comprising SEQ ID NO:97.

In some embodiments, an anti-B7-H4 antibody comprises a heavy chain variable region comprising SEQ ID NO:166. In some embodiments, an anti-B7-H4 antibody comprises a light chain variable region comprising SEQ ID NO:97. In some embodiments, an anti-B7-H4 antibody comprises a heavy chain variable region comprising SEQ ID NO:166 and a light chain variable region comprising SEQ ID NO:97.

In some embodiments, an anti-B7-H4 antibody comprises a heavy chain variable region comprising SEQ ID NO:167. In some embodiments, an anti-B7-H4 antibody comprises a light chain variable region comprising SEQ ID NO:106. In some embodiments, an anti-B7-H4 antibody comprises a heavy chain variable region comprising SEQ ID NO:167 and a light chain variable region comprising SEQ ID NO:106.

In some embodiments, an anti-B7-H4 antibody comprises a heavy chain variable region comprising SEQ ID NO:168. In some embodiments, an anti-B7-H4 antibody comprises a light chain variable region comprising SEQ ID NO:97. In some embodiments, an anti-B7-H4 antibody comprises a heavy chain variable region comprising SEQ ID NO:168 and a light chain variable region comprising SEQ ID NO:97.

In some embodiments, an anti-B7-H4 antibody comprises a heavy chain variable region comprising SEQ ID NO:169. In some embodiments, an anti-B7-H4 antibody comprises a light chain variable region comprising SEQ ID NO:97. In some embodiments, an anti-B7-H4 antibody comprises a heavy chain variable region comprising SEQ ID NO:169 and a light chain variable region comprising SEQ ID NO:97.

In some embodiments, an anti-B7-H4 antibody comprises a heavy chain variable region comprising SEQ ID NO:170. In some embodiments, an anti-B7-H4 antibody comprises a light chain variable region comprising SEQ ID NO:102. In some embodiments, an anti-B7-H4 antibody comprises a heavy chain variable region comprising SEQ ID NO:170 and a light chain variable region comprising SEQ ID NO:102.

In some embodiments, an anti-B7-H4 antibody comprises a heavy chain variable region comprising SEQ ID NO:171. In some embodiments, an anti-B7-H4 antibody comprises a light chain variable region comprising SEQ ID NO:97. In some embodiments, an anti-B7-H4 antibody comprises a heavy chain variable region comprising SEQ ID NO: 171 and a light chain variable region comprising SEQ ID NO:97.

In some embodiments, an anti-B7-H4 antibody comprises a heavy chain variable region comprising SEQ ID NO:172. In some embodiments, an anti-B7-H4 antibody comprises a light chain variable region comprising SEQ ID NO:114. In some embodiments, an anti-B7-H4 antibody comprises a heavy chain variable region comprising SEQ ID NO:172 and a light chain variable region comprising SEQ ID NO:114.

In some embodiments, an anti-B7-H4 antibody comprises a heavy chain variable region comprising SEQ ID NO:173. In some embodiments, an anti-B7-H4 antibody comprises a light chain variable region comprising SEQ ID NO:97. In some embodiments, an anti-B7-H4 antibody comprises a heavy chain variable region comprising SEQ ID NO:173 and a light chain variable region comprising SEQ ID NO:97.

In some embodiments, an anti-B7-H4 antibody comprises a heavy chain variable region comprising SEQ ID NO:174. In some embodiments, an anti-B7-H4 antibody comprises a light chain variable region comprising SEQ ID NO:97. In some embodiments, an anti-B7-H4 antibody comprises a heavy chain variable region comprising SEQ ID NO:174 and a light chain variable region comprising SEQ ID NO:97.

In some embodiments, an anti-B7-H4 antibody comprises a heavy chain variable region comprising SEQ ID NO:175. In some embodiments, an anti-B7-H4 antibody comprises a light chain variable region comprising SEQ ID NO:97. In some embodiments, an anti-B7-H4 antibody comprises a heavy chain variable region comprising SEQ ID NO:175 and a light chain variable region comprising SEQ ID NO:97.

In some embodiments, an anti-B7-H4 antibody comprises a heavy chain variable region comprising SEQ ID NO:176. In some embodiments, an anti-B7-H4 antibody comprises a light chain variable region comprising SEQ ID NO:101. In some embodiments, an anti-B7-H4 antibody comprises a heavy chain variable region comprising SEQ ID NO:176 and a light chain variable region comprising SEQ ID NO:101.

In some embodiments, an anti-B7-H4 antibody comprises a heavy chain variable region comprising SEQ ID NO:177. In some embodiments, an anti-B7-H4 antibody comprises a light chain variable region comprising SEQ ID NO:101. In some embodiments, an anti-B7-H4 antibody comprises a heavy chain variable region comprising SEQ ID NO:177 and a light chain variable region comprising SEQ ID NO:101.

In some embodiments, an anti-B7-H4 antibody comprises a heavy chain variable region comprising SEQ ID NO:178. In some embodiments, an anti-B7-H4 antibody comprises a light chain variable region comprising SEQ ID NO: 115. In some embodiments, an anti-B7-H4 antibody comprises a heavy chain variable region comprising SEQ ID NO:178 and a light chain variable region comprising SEQ ID NO:115.

In some embodiments, an anti-B7-H4 antibody comprises a heavy chain variable region comprising SEQ ID NO:179. In some embodiments, an anti-B7-H4 antibody comprises a light chain variable region comprising SEQ ID NO:98. In some embodiments, an anti-B7-H4 antibody comprises a heavy chain variable region comprising SEQ ID NO:179 and a light chain variable region comprising SEQ ID NO:98.

In some embodiments, an anti-B7-H4 antibody comprises a heavy chain variable region comprising SEQ ID NO:125. In some embodiments, an anti-B7-H4 antibody comprises a light chain variable region comprising SEQ ID NO:116. In some embodiments, an anti-B7-H4 antibody comprises a heavy chain variable region comprising SEQ ID NO:125 and a light chain variable region comprising SEQ ID NO:116.

In some embodiments, an anti-B7-H4 antibody comprises a heavy chain variable region comprising SEQ ID NO:180. In some embodiments, an anti-B7-H4 antibody comprises a light chain variable region comprising SEQ ID NO:102. In some embodiments, an anti-B7-H4 antibody comprises a heavy chain variable region comprising SEQ ID NO:180 and a light chain variable region comprising SEQ ID NO:102.

In some embodiments, an anti-B7-H4 antibody comprises a heavy chain variable region comprising SEQ ID NO:181. In some embodiments, an anti-B7-H4 antibody comprises a light chain variable region comprising SEQ ID NO:97. In some embodiments, an anti-B7-H4 antibody comprises a heavy chain variable region comprising SEQ ID NO:181 and a light chain variable region comprising SEQ ID NO:97.

In some embodiments, an anti-B7-H4 antibody comprises a heavy chain variable region comprising SEQ ID NO:182. In some embodiments, an anti-B7-H4 antibody comprises a light chain variable region comprising SEQ ID NO:97. In some embodiments, an anti-B7-H4 antibody comprises a heavy chain variable region comprising SEQ ID NO:182 and a light chain variable region comprising SEQ ID NO:97.

In some embodiments, an anti-B7-H4 antibody comprises a heavy chain variable region comprising SEQ ID NO:183. In some embodiments, an anti-B7-H4 antibody comprises a light chain variable region comprising SEQ ID NO:97. In some embodiments, an anti-B7-H4 antibody comprises a heavy chain variable region comprising SEQ ID NO:183 and a light chain variable region comprising SEQ ID NO:97.

In some embodiments, an anti-B7-H4 antibody comprises a heavy chain variable region comprising SEQ ID NO: 184. In some embodiments, an anti-B7-H4 antibody comprises a light chain variable region comprising SEQ ID NO:97. In some embodiments, an anti-B7-H4 antibody comprises a heavy chain variable region comprising SEQ ID NO:184 and a light chain variable region comprising SEQ ID NO:97.

In some embodiments, an anti-B7-H4 antibody comprises a heavy chain variable region comprising SEQ ID NO:185. In some embodiments, an anti-B7-H4 antibody comprises a light chain variable region comprising SEQ ID NO:97. In some embodiments, an anti-B7-H4 antibody comprises a heavy chain variable region comprising SEQ ID NO:185 and a light chain variable region comprising SEQ ID NO:97.

In some embodiments, an anti-B7-H4 antibody comprises a heavy chain variable region comprising SEQ ID NO:186. In some embodiments, an anti-B7-H4 antibody comprises a light chain variable region comprising SEQ ID NO:97. In some embodiments, an anti-B7-H4 antibody comprises a heavy chain variable region comprising SEQ ID NO:186 and a light chain variable region comprising SEQ ID NO:97.

In some embodiments, an anti-B7-H4 antibody comprises a heavy chain variable region comprising SEQ ID NO:145. In some embodiments, an anti-B7-H4 antibody comprises a light chain variable region comprising SEQ ID NO:117. In some embodiments, an anti-B7-H4 antibody comprises a heavy chain variable region comprising SEQ ID NO:145 and a light chain variable region comprising SEQ ID NO:117.

In some embodiments, an anti-B7-H4 antibody comprises a heavy chain variable region comprising SEQ ID NO:187. In some embodiments, an anti-B7-H4 antibody comprises a light chain variable region comprising SEQ ID NO:97. In some embodiments, an anti-B7-H4 antibody comprises a heavy chain variable region comprising SEQ ID NO:187 and a light chain variable region comprising SEQ ID NO:97.

In some embodiments, an anti-B7-H4 antibody comprises a heavy chain variable region comprising SEQ ID NO:188. In some embodiments, an anti-B7-H4 antibody comprises a light chain variable region comprising SEQ ID NO:118. In some embodiments, an anti-B7-H4 antibody comprises a heavy chain variable region comprising SEQ ID NO:188 and a light chain variable region comprising SEQ ID NO:118.

In some embodiments, an anti-B7-H4 antibody comprises a heavy chain variable region comprising SEQ ID NO:189. In some embodiments, an anti-B7-H4 antibody comprises a light chain variable region comprising SEQ ID NO:97. In some embodiments, an anti-B7-H4 antibody comprises a heavy chain variable region comprising SEQ ID NO:189 and a light chain variable region comprising SEQ ID NO:97.

In some embodiments, an anti-B7-H4 antibody comprises a heavy chain variable region comprising SEQ ID NO:190. In some embodiments, an anti-B7-H4 antibody comprises a light chain variable region comprising SEQ ID NO:97. In some embodiments, an anti-B7-H4 antibody comprises a heavy chain variable region comprising SEQ ID NO:190 and a light chain variable region comprising SEQ ID NO:97.

In some embodiments, an anti-B7-H4 antibody comprises a heavy chain variable region comprising SEQ ID NO:178. In some embodiments, an anti-B7-H4 antibody comprises a light chain variable region comprising SEQ ID NO:97. In some embodiments, an anti-B7-H4 antibody comprises a heavy chain variable region comprising SEQ ID NO:178 and a light chain variable region comprising SEQ ID NO:97.

In some embodiments, the anti-B7-H4 antibody is an antibody fragment. Fragments of the antibodies described herein (e.g., Fab, Fab′, F(ab′)2, Facb, and Fv) may be prepared by any suitable methods known in the art, such as proteolytic digestion of intact antibodies. For example, antibody fragments can be obtained by treating the whole antibody with an enzyme such as papain, pepsin, or plasmin. Papain digestion of whole antibodies produces F(ab)₂ or Fab fragments; pepsin digestion of whole antibodies yields F(ab′)2 or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Plueckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)₂ fragments (Carter et al., Bio/Technology, 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046.

In some embodiments, the anti-B7-H4 antibody is a minibody. Minibodies of anti-B7-H4 antibodies include diabodies, single chain (scFv), and single-chain (Fv)₂ (sc(Fv)₂).

A “diabody” is a bivalent minibody constructed by gene fusion (see, e.g., Holliger. P. et al., Proc. Natl. Acad. Sci. U.S.A., 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites. The “diabody” technology also provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. An scFv is a single-chain polypeptide antibody obtained by linking the VH and VL with a linker (see, e.g., Huston et al., Proc. Natl. Acad. Sci. U.S.A., 85:5879-5883 (1988); and Plickthun, “The Pharmacology of Monoclonal Antibodies” Vol. 113, Ed Resenburg and Moore, Springer Verlag, New York, pp. 269-315, (1994)). The order of VHs and VLs to be linked is not particularly limited, and they may be arranged in any order. Examples of arrangements include: [VH] linker [VL]; or [VL] linker [VH]. The heavy chain variable domain and light chain variable domain in an scFv may be derived from any anti-B7-H4 antibody described herein.

An sc(Fv)₂ is a minibody in which two VHs and two VLs are linked by a linker to form a single chain (Hudson, et al., J. Immunol. Methods, (1999) 231: 177-189 (1999)). An sc(Fv)₂ can be prepared, for example, by connecting scFvs with a linker. The sc(Fv)₂ of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however the order of the two VHs and two VLs is not limited to the above arrangement, and they may be arranged in any order.

In some instances, the anti-B7-H4 antibody is a bispecific antibody. Bispecific antibodies are antibodies that have binding specificities for at least two different epitopes. Exemplary bispecific antibodies may bind to two different epitopes of the B7-H4 protein. Other such antibodies may combine a B7-H4 binding site with a binding site for another protein. Bispecific antibodies can be prepared as full length antibodies or fragments thereof (e.g., F(ab′)₂ bispecific antibodies, sc(Fv)₂ bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

In some instances, the anti-B7-H4 antibody is a multivalent antibody. A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD₂-(X₂)ₙ-Fc, wherein VD1 is a first variable domain, VD2 is a second variable domain, Fc is a polypeptide chain of an Fc region, X1 and X2 represent an amino acid or peptide spacer, and n is 0 or 1.

In some instances, the anti-B7-H4 antibody is a conjugated antibody. The antibodies disclosed herein may be conjugated antibodies, which are bound to various molecules including macromolecular substances such as polymers (e.g., polyethylene glycol (PEG), polyethylenimine (PET) modified with PEG (PEI-PEG), polyglutamic acid (PGA) (N-(2-Hydroxypropyl) methacrylamide (HPMA) copolymers), hyaluronic acid, radioactive materials (e.g., ⁹Y, ¹³¹I), fluorescent substances, luminescent substances, haptens, enzymes, metal chelates, drugs, and toxins (e.g., calcheamicin, Pseudomonas exotoxin A, ricin (e.g., deglycosylated ricin A chain)).

In one embodiment, to improve the cytotoxic actions of anti-B7-H4 antibodies and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (thus, the use of murine sequences, and IgG3 or IgG4 isotypes).

In certain embodiments, an anti-B7-H4 antibody is modified with a moiety that improves its stabilization and/or retention in circulation, e.g., in blood, serum, or other tissues, e.g., by at least 1.5, 2, 5, 10, or 50 fold. For example, the anti-B7-H4 antibody can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-B7-H4 antibody can be conjugated to a water soluble polymer, e.g., a hydrophilic polyvinyl polymer, e.g., polyvinylalcohol or polvvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies, respectively, or the lower molecular weight forms thereof described herein. Methods for modifying antibodies are well known in the art (e.g., U.S. Pat. Nos. 5,057,313 and 5,156,840).

### Methods of Producing Antibodies

Antibodies may be produced in bacterial or eukaryotic cells. Some antibodies, e.g., Fabs, can be produced in bacterial cells, e.g., E. coli cells. Antibodies can also be produced in eukaryotic cells such as transformed cell lines (e.g., CHO, 293E, COS). In addition, antibodies (e.g., scFvs) can be expressed in a yeast cell such as Pichia (see, e.g., Powers et al., J Immunol Methods. 251:123-35 (2001)), Hanseula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5a, HB101, or XL1-Blue is used as a host, the vector must have a promoter, for example, a lacZ promoter (Ward et al., 341:544-546 (1989), araB promoter (Better et al., Science. 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601 621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-B7-H4 antibody is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation. SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. The present disclosure also provides transgenic animals comprising one or more of the nucleic acids described herein.

### Polynucleotides, Expression Vectors, and Cells

The disclosure also provides polynucleotides and vectors encoding an anti-B7-H4 antibody or portion thereof (e.g., VH, VL, HC, or LC) described herein. The polynucleotides of the disclosure can be in the form of RNA or in the form of DNA. In some instances, the polynucleotide is DNA. In some instances, the polynucleotide is complementary DNA (cDNA). In some instances, the polynucleotide is RNA. In some embodiments, a polynucleotide described herein is isolated.

In some instances, the polynucleotide encodes a VH comprising the VH CDR1, VH CDR2, and VH CDR3 of any antibody described herein (see, e.g., Tables 1, 4, and 5). In some instances, the polynucleotide encodes a VL comprising the VL CDR1, VL CDR2, and VL CDR3 of any antibody described herein (see, e.g., Tables 1, 4, and 5). In some instances, the polynucleotide encodes a heavy chain comprising a VH comprising the VH CDR1, VH CDR2, and VH CDR3 of any antibody described herein (see, e.g., Tables 1, 4, and 5). In some instances, the polynucleotide encodes a light chain comprising a VL comprising the VL CDR1, VL CDR2, and VL CDR3 of any antibody described herein (see, e.g., Tables 1, 4, and 5). In some instances, the polynucleotide is operably linked to a promoter.

In some instances, the polynucleotide comprises: (i) a first nucleic acid sequence encoding a first polypeptide, wherein the first polypeptide comprises a VH comprising the VH CDR1, VH CDR2, and VH CDR3 of any antibody described herein (see, e.g., Tables 1, 4, and 5); and (ii) a second nucleic acid sequence encoding a second polypeptide, wherein the second polypeptide comprises a VL comprising the VL CDR1, VL CDR2, and VL CDR3 of any antibody described herein (see, e.g., Tables 1, 4, and 5). In some instances, the polynucleotide comprises: (i) a first nucleic acid sequence encoding a first polypeptide, wherein the first polypeptide comprises a heavy chain comprising a VH comprising the VH CDR1, VH CDR2, and VH CDR3 of any antibody described herein (see, e.g., Tables 1, 4, and 5); and (ii) a second nucleic acid sequence encoding a second polypeptide, wherein the second polypeptide comprises a light chain comprising a VL comprising the VL CDR1, VL CDR2, and VL CDR3 of any antibody described herein (see, e.g., Tables 1, 4, and 5). In some instances, the first nucleic acid is operably linked to a first promoter and the second nucleic acid is operably linked to a second promoter.

In some instances, the polynucleotide encodes a VH described herein (see, e.g., Table 5) or a variant thereof. In some instances, the polynucleotide encodes a polypeptide comprising an amino acid sequence having at least 80%, at least 85%, at least 90%, at least 95%, at least 97%, or 100% identity to the amino acid sequence set forth in any one of SEQ ID NOs: 119-190. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 119-190. In some instances, the polynucleotide encodes a polypeptide comprising the amino acid sequence set forth in any one of SEQ ID NOs: 119-190. In some instances, the polynucleotide is operably linked to a promoter.

In some instances, the polynucleotide encodes a VL described herein (see, e.g., Table 5) or a variant thereof. In some instances, the polynucleotide encodes a polypeptide comprising an amino acid sequence having at least 80%, at least 85%, at least 90%, at least 95%, at least 97%, or 100% identity to the amino acid sequence set forth in any one of SEQ ID NOs: 96-118. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 96-118. In some instances, the polynucleotide encodes a polypeptide comprising the amino acid sequence set forth in any one of SEQ ID NOs: 96-118. In some instances, the polynucleotide is operably linked to a promoter.

In some instances, the polynucleotide comprises: (i) a first nucleic acid encoding a first polypeptide, wherein the first polypeptide comprises a VH described herein (see, e.g., Table 5) or a variant thereof; and (ii) a second nucleic acid encoding a second polypeptide, wherein the second polypeptide comprises a VL described herein (see, e.g., Table 5) or a variant thereof. In some instances, the polynucleotide comprises: (i) a first nucleic acid sequence encoding a first polypeptide, wherein the first polypeptide comprises an amino acid sequence having at least 80%, at least 85%, at least 90%, at least 95%, at least 97%, or 100% identity to the amino acid sequence set forth in any one of SEQ ID NOs: 119-190, and (ii) a second nucleic acid sequence encoding a second polypeptide, wherein the second polypeptide comprises an amino acid sequence having at least 80%, at least 85%, at least 90%, at least 95%, at least 97%, or 100% identity to the amino acid sequence set forth in any one of SEQ ID NOs: 96-118. In some instances, the polynucleotide comprises: (i) a first nucleic acid sequence encoding a first polypeptide, wherein the first polypeptide comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 119-190; and (ii) a second nucleic acid sequence encoding a second polypeptide, wherein the second polypeptide comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs: 96-118. In some instances, the first nucleic acid encodes the amino acid sequence set forth in any one of SEQ ID NOs: 119-190 and the second nucleic acid encodes the amino acid sequence set forth in any one of SEQ ID NOs: 96-118. In some instances, the first nucleic acid is operably linked to a first promoter and the second nucleic acid is operably linked to a second promoter.

Also provided herein are expression vectors encoding the anti-B7-H4 antibodies or portions thereof (e.g., VH, VL. HC, and/or LC) described herein. Also provided herein are expression vectors comprising one or more polynucleotides described herein. Various types of expression vectors are known in the art and described herein.

Also provided herein are cells comprising the anti-B7-H4 antibodies described herein. Also provided herein are cells comprising one or more polynucleotides described herein. Also provided herein are cells comprising one or more expression vectors described herein. Various types of cells are known in the art and described herein.

### Indications

The anti-B7-H4 antibodies of the present disclosure can modulate the activity of B7-H4. Accordingly, the antibodies or compositions described herein can be used in methods of inhibiting activity of B7-H4 in an individual/patient in need of the inhibition comprising administering an effective amount of an antibody described herein. In some embodiments, modulating is inhibiting. In some embodiments, the contacting is in vivo. In some embodiments, the contacting is ex vivo or in vitro.

Another aspect of the present disclosure pertains to methods of treating a B7-H4-associated disease or disorder in an individual (e.g., patient) comprising administering to the individual in need of such treatment a therapeutically effective amount or dose of one or more antibodies of the present disclosure or a pharmaceutical composition thereof. A B7-H4-associated disease or disorder can include any disease, disorder or condition that is directly or indirectly linked to expression or activity of B7-H4, including overexpression and/or abnormal activity levels.

Another aspect of the present disclosure pertains to methods of treating a cancer in an individual (e.g., patient) comprising administering to the individual in need of such treatment a therapeutically effective amount or dose of one or more antibodies of the present disclosure or a pharmaceutical composition thereof. In some examples, the cancer is a B7-H4-expressing cancer. Methods of determining whether a cancer is a B7-H4-expressing cancer are known in the art. For instance, the presence of B7-H4 on the surface of tumor cells can be determined using suitable immunohistochemistry methods known in the art.

In some embodiments, anti-B7-H4 antibodies disclosed herein can be used to treat, alone or in combination with other therapies, a cancer such as ovarian cancer, breast cancer, endometrial cancer, bladder cancer, esophageal cancer, oral squamous cell carcinoma, brain cancer, prostate cancer, pancreatic cancer, cervical cancer, skin cancer, lung cancer, gastric cancer, and renal cell carcinoma.

Non-limiting examples of other therapies include a Janus tyrosine kinase (JAK) inhibitor (e.g., ruxolitinib and itaticinib), a phosphoinositide 3-kinase (PI3K) inhibitor (e.g., parsaclisib), a standard of care therapy (e.g., a chemotherapy, a radiation therapy, a surgical therapy, an immunotherapy), or a combination thereof.

Non-limiting examples of JAK inhibitors for use as described herein are provided in U.S. Pat. Nos. 7,335,667; 9,359,358; 8,691,807; 9,181,271; and 9,034,884, each of which is incorporated herein by reference in its entirety.

Non-limiting examples of PI3K inhibitors for use as described herein are provided in U.S. Pat. Nos. 9,108,984; 9,062,055; 8,759,359; and 9,434,746, each of which is incorporated herein by reference in its entirety.

The terms “individual” or “patient” or “subject”, used interchangeably, refer to any animal, including mammals, preferably mice, rats, other rodents, rabbits, dogs, cats, swine, cattle, sheep, horses, or primates, and most preferably humans (i.e., a human subject).

The phrase “therapeutically effective amount” refers to the amount of active antibody or pharmaceutical agent that elicits the biological response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder or one or more symptoms thereof in an individual who is experiencing or displaying the pathology or symptomatology of the disease, condition or disorder, and can include arresting further development of the pathology, progression of existing symptoms and/or occurrence of new symptomatology; and (2) ameliorating the disease; e.g., ameliorating a disease, condition or disorder or one or more symptoms thereof in an individual who is experiencing or displaying the pathology or symptomatology of the disease, condition or disorder, and can include reversing or arresting the pathology or existing symptoms and/or preventing the onset of new symptomatology, such as decreasing the severity of disease. Inhibiting or ameliorating the disease can also include the killing of cancer or neoplastic cells in the individual (cytotoxic effect) or the arresting of cancer or neoplastic cell growth (cytostatic effect), or preventing or slowing the metastasis of cancer or neoplastic cells.

In some embodiments, the antibodies of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Pharmaceutical Compositions

An anti-B7-H4 antibody described herein can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see, e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the anti-B7-H4 antibody may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

### Uses

The anti-B7-H4 antibody can be administered to a subject, e.g., a subject in need thereof, for example, a human subject, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the antibody can also be tailored for the individual case, e.g., by monitoring the subject, e.g., using tomographic imaging, e.g., to visualize a tumor.

The antibody can be administered as a fixed dose, or in a mg/kg patient weight dose. The dose can also be chosen to reduce or avoid production of antibodies against the anti-B7-H4 antibody. Dosage regimens are adjusted to provide the desired response, e.g., a therapeutic response or a combinatorial therapeutic effect. Generally, doses of the anti-B7-H4 antibody (and optionally a second agent) can be used in order to provide a subject with the agent in bioavailable quantities.

Dosage unit form or “fixed dose” or “flat dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion.

Exemplary fixed doses include about 375 mg, about 500 mg and about 750 mg. In some embodiments, with respect to doses or dosages, the term “about” is intended to denote a range that is ±10% of a recited dose, such that, for example, a dose of about 375 mg will be between 337.5 mg and 412.5 mg.

In some embodiments, for administration of any of the anti-B7-H4 antibodies described herein, an initial dosage can be about 2 mg/kg. In some embodiments, a typical daily dosage can range from about any of 0.1 μg/kg to 3 μg/kg to 20 μg/kg to 300 μg/kg to 3 mg/kg, to 30 mg/kg to 100 mg/kg or more. For repeated administrations over several days or longer, depending on the condition, the treatment can be sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing can be performed one to four times per week. In some embodiments, dosing ranging from about 3 μg/mg to about 2 mg/kg (e.g., about 3 μg/kg, about 10 μg/kg, about 30 μg/kg, about 100 μg/kg, about 300 μg/kg, about 1 mg/kg, or about 2 mg/kg) can be used. In some embodiments, dosing frequency is once every week, every 2 weeks, every 3 weeks, every 4 weeks, every 5 weeks, every 6 weeks, every 7 weeks, every 8 weeks, every 9 weeks, every 10 weeks, or longer.

Anti-B7-H4 antibodies described herein can be used for detecting B7-H4 and/or evaluating a cancer (e.g., diagnosis and/or prognosis of a cancer). Evaluation can include identifying a subject as being at risk for or having a cancer. Evaluation can also include monitoring treatment of a disease such as evaluating the effectiveness of a treatment for a cancer.

In some embodiments, anti-B7-H4 antibodies described herein can be used to detect B7-H4 in a sample (e.g., a blood sample, a tissue sample) obtained from a subject (e.g., a human patient). For example, methods for detecting B7-H4 comprise contacting the anti-B7-H4 antibody with a sample suspected of containing B7-H4, and detecting binding of the antibody to B7-H4 in the sample. In general, the term “contacting” refers to an exposure of the anti-B7-H4 antibody with the sample under conditions and for a time sufficient for the formation of a complex between the anti-B7-H4 antibody and B7-H4 in the sample, if any. The anti-B7-H4 antibodies may be used with or without modification, and can be labeled by covalent or non-covalent attachment of a detectable label. A wide variety of detectable labels are known in the art and can be used.

In some embodiments, anti-B7-H4 antibodies described herein can be used to detect B7-H4 in a sample obtained from a subject and the level of B7-H4 is then compared to a reference level to determine whether the subject has or is at risk for a cancer. By comparing the level of B7-H4 in a sample obtained from a subject to the reference level, it can be determined as to whether the subject has or is at risk for a disease. For example, if the level of B7-H4 in the sample from the subject is elevated as compared to the reference value, the subject is identified as having or at risk for a cancer.

As used herein, “an elevated level or a level above a reference value” means that the level of B7-H4 is higher than a reference value, such as a pre-determined threshold or a level of B7-H4 in a control sample.

An elevated level of B7-H4 includes a B7-H4 level that is, for example, 50%, 60%, 70%, 80%, 90%, 100%, 150%, 200%, 300%, 400%, 500% or more above than a reference value. In some embodiments, an elevated level of B7-H4 includes a B7-H4 level that is at least 1.1, 1.2, 1.3, 1.4, 1.5, 1.6, 1.7, 1.8, 1.9, 2, 2.5, 3, 3.5, 4, 4.5, 5, 6, 7, 8, 9, 10, 25, 50, 75, 100, 150, 200, 300, 400, 500, 1000-fold or more higher than the level of the reference level. An elevated level of B7-H4 also includes increasing a phenomenon from a zero state (e.g., no or undetectable B7-H4 in a sample) to a non-zero state (e.g., some or detectable B7-H4 in a sample).

Methods described herein involve determining the level of B7-H4 in a sample from a subject, wherein an elevated level of B7-H4 in the sample compared to a reference level predicts whether a subject is likely to develop a neurological disease. In some embodiments, a control sample is obtained from a healthy subject or population of healthy subjects. As used herein, a healthy subject is a subject that is apparently free of a cancer at the time the level of B7-H4 is measured or a subject that has no history of cancer. The control level as described herein can be determined by methods described herein or by methods known in the art.

The control level can also be a predetermined level. The predetermined level or score can be a single cut-off (threshold) value, such as a median or mean, or a level or score that defines the boundaries of an upper or lower quartile, tertile, or other segment of a population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

The disclosure also provides a kit comprising one or more containers of an anti-B7-H4 antibody or a pharmaceutical formulation thereof, optionally with one or more other prophylactic or therapeutic agents useful for the treatment of a disease or disorder, and optionally with instructions for using the anti-B7-H4 antibody or a pharmaceutical formulation thereof.

The instructions relating to the use of an anti-B7-H4 antibody for treatment of a subject generally include information as to dosage, dosing schedule, and route of administration for the intended treatment. The containers can be unit doses, bulk packages (e.g., multi-dose packages) or sub-unit doses. Instructions supplied in the kits of the disclosure are typically written instructions on a label or package insert. The label package insert indicates that an anti-B7-H4 antibody is used for treating, delaying the onset, and/or alleviating a cancer.

In some embodiments, the kit comprises a detection agent (e.g., an antibody that binds the anti-B7-H4 antibody) for detecting binding of the anti-B7-H4 antibody to B7-H4. Instructions relating to use of an anti-B7-H4 antibody for detecting B7-H4 generally describe how to use the anti-B7-H4 contained in the kit for detecting B7-H4 in a subject or in a sample from a subject.

### Examples

The following examples are provided to better illustrate the claimed invention and are not to be interpreted as limiting the scope of the invention in any way. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Parental Antibody, Libraries Construction, and Selections (Phase I)

An antibody directed against B7-H4 was chosen to be affinity matured as described herein. The antibody, comprising a VH3 heavy chain and a Vλ3 light chain, was initially identified by biopanning a naïve human scFv phage display library (Erasmus, D'Angelo et al. 2021). Analysis of the CDRs revealed three sequence liabilities with the potential to affect downstream clinical development: two distinct aspartate isomerization sites, which can lead to chemical degradation and loss of potency (Sydow, Lipsmeier et al. 2014), in LCDR2 and LCDR3, and a GG nonspecificity motif (Kelly, Le et al. 2018) in HCDR2. The scFv affinity (KD) of the parental antibody was determined to be 7.4 nM (kₐ=4.5×10⁵ M⁻¹s⁻¹; k_{d}=3.3×10⁻³s⁻¹) by surface plasmon resonance.

Deep sequencing data (MiSeq and NovaSeq) from the same antibody library was used to identify CDRs that could be shuffled into the parental sequence for affinity maturation. For LCDR1-2, only CDR sequences coming from the VW3 germline family were considered, and for HCDR1-2, only sequences coming from the VH3 family were considered, assuming these sequences, since they belonged to the same germline gene families as the parental antibody, would be better tolerated and minimize potential structural disruption. For LCDR3, however, all λ light chain LCDR3 sequences were included, regardless of the specific family or germline since, in theory, the CDR3 region should be able to support a more diverse set of sequences. To minimize the chance of selecting antibodies with unfavorable developability profiles, sequences containing undesirable liabilities such as glycosylation sites and unpaired cysteines were eliminated (see Table 8 for the full list of excluded liabilities). Finally, the identified CDRs were generated with flanking framework sequences matching the parental antibody and produced using array-based DNA synthesis (Agilent). This allowed rescue of the full diversity at each individual CDR site by using framework primers.

To decrease the initial combinatorial diversity and fully explore the potential at each site, three separate libraries were created: L1L2, H1H2, and L3 (Table 2). These allowed generation of more transformants than the maximum combinatorial diversities for each library (theoretical diversity ranging from 1.7×10⁵ to 3.2×10⁷). Also, by fixing at least four parental CDRs, including the all-important HCDR3, in each library, search space was decreased, increasing the chances of finding new variants binding to the antigen in the same way, essential to retaining biological activity. In addition to the incorporated CDR diversity, each library also included the parental CDR sequences at the same abundance as the other introduced CDRs, even if they contained sequence liabilities. This was to ensure retained activity in the case that particular parental CDR sequences were essential for binding.

TABLE 2
Number of different sequences introduced in each CDR position of
light and heavy chain. Theoretical diversity is calculated by the
combinatorial potential of the CDRs and the reported number of
transformants correspond to the yeast display libraries created.

| Family | Region | CDRs | Library | Diversity | Transformants |
|---|---|---|---|---|---|
| Vλ3 | LCDR1 | 1,155 | L1L2 | 498,960 | 1.26 × 10⁷ |
| | LCDR2 | 432 | | | |
| Vλ | LCDR3 | 166,196 | L3 | 166,196 | 1.23 × 10⁷ |
| VH3 | HCDR1 | 9,874 | H1H2 | 31,685,666 | 7.4 × 10⁷ |
| | HCDR2 | 3,209 | | | |

Libraries were assembled by combining the newly produced CDR pools with the remaining parts of the scFv. For example, the L1L2 library was assembled by (1) amplifying the LCDR1 and LCDR2 with the flanking frameworks from the synthetic oligo pool, (2) amplifying the remaining parts of the scFv from the parental clones, (3) assembling the produced fragments by overlap PCR, and (4) transforming the produced scFv cassettes into S. cerevisiae along with the digested yeast display vector.

Two different selection strategies were used in this phase: equilibrium selection and kinetic selection (Boder and Wittrup 1998, Boder, Midelfort et al. 2000). Equilibrium selection, the more traditional approach, was performed by incubating the scFv-displaying yeast cells with a defined concentration of labeled antigen (biotinylated, in this case) and sorting labeled cells immediately after reaching equilibrium. Incubations are often performed with decreasing antigen concentrations as the selections round progress. However, decreasing the antigen concentration cannot be carried out indefinitely since the displayed antibodies on the yeast surface will deplete antigen from the solution before reaching equilibrium (VanAntwerp and Wittrup 2000). Avoiding this requires the use of large and impractical incubation volumes with small numbers of cells, to ensure minimal antigen is removed and the effective concentration remains constant. An alternative to equilibrium selection is kinetic selection: scFv-displaying yeast cells are incubated with the labeled antigen, washed, incubated with unlabeled antigen to select only clones with stable binding to the antigen (slow off-rate—k_{d}). The unlabeled antigen is used to prevent rebinding of the displaced labeled antigen. After a defined period, cells still bound to the labeled antigen are sorted.

An initial flow cytometric assessment of the libraries was performed using decreasing antigen concentrations. The light chain libraries, L1L2 and L3, showed a small population binding to the antigen even at the highest concentration used: at 100 nM binding populations of 3% and 1.5% respectively, while for the H1H2 heavy chain library significant binding could be observed from 1.2 nM (2.9% of the population) to 100 nM (8.3% of the population), suggesting higher improvement potential for the heavy chain CDRs as opposed to the light chains for this particular clone.

Given the size of each of these libraries, first and second rounds of selection were performed using magnetic-assisted cell sorting (MACS) at antigen concentrations of 10 nM and 1 nM, respectively. This allowed labeling and sorting of a larger number of cells than what would be practical using a flow cytometer. For subsequent rounds, fluorescence-activated cell sorting (FACS) was used to enable more precise sorting of the cells of interest.

After the first three rounds of equilibrium sorting, two rounds of kinetic sorting with 4 hours of competition were performed for the L3 and H1H2 libraries. Only one 4 hour kinetic sort was performed for the L1L2 library, and the final round was performed as a negative sort, where the population was incubated only with the secondary reagents and negative cells were sorted—this was done out of concern for possible enrichment of polyreactive antibodies from this library since weak positivity was identified in the absence of antigen in the kinetic sort of this library (but not the others).

An assessment of the population obtained for each of these libraries after five rounds show that in all cases a significant improvement of affinity can be observed: all generated populations show significant binding to the antigen even 4 hours after the labeled antigen has been removed from the solution, a time at which the parental antibody shows minimal binding. Even though affinities of individual clones were not assessed at this stage, it is not unreasonable to assume that, given the yeast display staining profile, one could already find binders with satisfactory affinities at this stage, depending on the requirements of the project.

### Example 2: Combination Libraries Construction and Selections (Phase 2)

After selecting CDRs at each position in the three initial libraries that showed improved binding, the selection outputs were combined to further improve binding towards still slower off-rates. Two combination libraries were assembled by PCR: the first (Combo 1; 2.27×10⁸ transformants) was created by combining the output of all three libraries (L1L2, L3, and H1H2), while the second (Combo 2: 1.04×10⁸ transformants) omitted the L1L2 output and used the parental CDRs at LCDR1 and LCDR2. Again, an approach combining equilibrium and kinetic sorts was used with two successive rounds of kinetic sorting at 4 hours and 16 hours with unlabeled antigen respectively, followed by a final equilibrium round at 0.1 nM antigen concentration.

An initial assessment showed that immediately after transformation the libraries already showed binding to the antigen even at 0.1 nM—51.8% and 62.9% of the population for Combo 1 and 2, respectively. After three rounds of sorting, the difference between the combination libraries and the parental antibody is striking: at 0.1 nM >80% of the yeast population bound to the antigen for both libraries as opposed to 55% for the parental antibody; when stained with 1 nM of antigen, and destained for 4 hours, both combination libraries show very little signal loss compared to the staining at I nM of antigen with no destaining period, whereas the parental completely lost binding under these conditions.

### Example 3: Sequence, Epitope, and Affinity Screening of Affinity Matured Clones

To evaluate the clones obtained, the populations obtained after the 3rd round of selection of the combination libraries were converted to an scFv-Fc format to facilitate affinity screening, 92 clones (45 from Combo 1 and 47 from Combo 2) were sequenced and 81 unique sequences were identified: 38 from Combo 1, 39 from Combo 2, and 4 in both libraries. These unique sequences were often formed by different combinations of the same CDRs found in LCDR1-3 and HCDR1-2 (Table 3). Interestingly, the heavy chain CDRs were more diverse than the light CDRs. This relates to the observed binding pattern of the first naïve libraries that showed higher binding signal for the H1H2 library, suggesting that the heavy CDRs indeed were more tolerant to sequence changes.

TABLE 3
Number of unique CDRs found in the 92
sequenced clones (81 unique found).

| Region | Unique |
|---|---|
| LCDR1 | 12 |
| LCDR2 | 5 |
| LCDR3 | 5 |
| HCDR1 | 20 |
| HCDR2 | 45 |

The amino acid sequences of the six CDRs for each of the 81 unique clones are shown in Table 4. The VH and VL sequences of each clone are shown in Table 5. Sequences of the parental clone (H6.1) are also shown in Table 4 and Table 5.

TABLE 4
CDR sequences.

| Clone | LCDR1 | LCDR2 | LCDR3 | HCDR1 | HCDR2 | HCDR3 |
|---|---|---|---|---|---|---|
| Parental | NIGSKS | DDSDRPS | QVWDSRTYV | GFTFSSYS | ISGSGGST | ARKRWYGMDV |
| H6.1 | (SEQ ID NO: 1) | (SEQ ID NO: 13) | (SEQ ID NO: 18) | (SEQ ID NO: 23) | (SEQ ID NO: 44) | (SEQ ID NO: 90) |
| A01 | NIGSKS | DDSDRPS | QVWDRGMI | GFTFSNKA | ISGSGATT | ARKRWYGMDV |
| | (SEQ ID NO: 1) | (SEQ ID NO: 13) | (SEQ ID NO: 19) | (SEQ ID NO: 24) | (SEQ ID NO: 45) | (SEQ ID NO: 90) |
| A02 | NIGSKS | DDSDRPS | QVWDRGMI | GFTFTSRA | ISYEGRTQ | ARKRWYGMDV |
| | (SEQ ID NO: 1) | (SEQ ID NO: 13) | (SEQ ID NO: 19) | (SEQ ID NO: 25) | (SEQ ID NO: 46) | (SEQ ID NO: 90) |
| A04 | NIGSKS | DDSDRPS | QVWDRGMI | GFTFSSRS | ISGSGDTT | ARKRWYGMDV |
| | (SEQ ID NO: 1) | (SEQ ID NO: 13) | (SEQ ID NO: 19) | (SEQ ID NO: 26) | (SEQ ID NO: 47) | (SEQ ID NO: 90) |
| A05 | NIGSKS | DDSDRPS | QVWDRGMI | GFTYSSRA | ISGSGSNA | ARKRWYGMDV |
| | (SEQ ID NO: 1) | (SEQ ID NO: 13) | (SEQ ID NO: 19) | (SEQ ID NO: 27) | (SEQ ID NO: 48) | (SEQ ID NO: 90) |
| A06 | NIVTKS | DDSDRPS | QVWDRGMI | GFTFGDRA | ISGTGATT | ARKRWYGMDV |
| | (SEQ ID NO: 2) | (SEQ ID NO: 13) | (SEQ ID NO: 19) | (SEQ ID NO: 28) | (SEQ ID NO: 49) | (SEQ ID NO: 90) |
| A07 | NIGSKS | DDSDRPS | QVWDSRTYV | GFTFSSRS | ISGSGSTV | ARKRWYGMDV |
| | (SEQ ID NO: 1) | (SEQ ID NO: 13) | (SEQ ID NO: 18) | (SEQ ID NO: 26) | (SEQ ID NO: 50) | (SEQ ID NO: 90) |
| A08 | NIGSKS | DDSDRPS | QVWDRGMI | GFTFGDRA | ISSSGTTS | ARKRWYGMDV |
| | (SEQ ID NO: 1) | (SEQ ID NO: 13) | (SEQ ID NO: 19) | (SEQ ID NO: 28) | (SEQ ID NO: 51) | (SEQ ID NO: 90) |
| A09 | NIGSKS | DDSDRPS | QVWDRGMI | GFTFSSRS | ISGSGLTS | ARKRWYGMDV |
| | (SEQ ID NO: 1) | (SEQ ID NO: 13) | (SEQ ID NO: 19) | (SEQ ID NO: 26) | (SEQ ID NO: 52) | (SEQ ID NO: 90) |
| A10 | NIGSKS | DDSDRPS | QVWDRGMI | GFTFSSRS | ISGTGYTT | ARKRWYGMDV |
| | (SEQ ID NO: 1) | (SEQ ID NO: 13) | (SEQ ID NO: 19) | (SEQ ID NO: 26) | (SEQ ID NO: 53) | (SEQ ID NO: 90) |
| A11 | NIGSKS | DDSDRPS | QVWDRGMI | GFTFSSRS | ISGSGSTV | ARKRWYGMDV |
| | (SEQ ID NO: 1) | (SEQ ID NO: 13) | (SEQ ID NO: 19) | (SEQ ID NO: 26) | (SEQ ID NO: 50) | (SEQ ID NO: 90) |
| A12 | NIGSKS | DDSDRPS | QVWDSRTYV | GFTFDNRA | ISSSGSTM | ARKRWYGMDV |
| | (SEQ ID NO: 1) | (SEQ ID NO: 13) | (SEQ ID NO: 18) | (SEQ ID NO: 29) | (SEQ ID NO: 54) | (SEQ ID NO: 90) |
| B01 | NIGVKS | DDSDRPS | QVWDSRTYV | GFTFSDRA | ISYEGRTQ | ARKRWYGMDV |
| | (SEQ ID NO: 3) | (SEQ ID NO: 13) | (SEQ ID NO: 18) | (SEQ ID NO: 30) | (SEQ ID NO: 46) | (SEQ ID NO: 90) |
| B02 | NIGVKS | DDSDRSP | QVWDSRTYV | GFTFSSRS | ISGSGSTV | ARKRWYGMDV |
| | (SEQ ID NO: 3) | (SEQ ID NO: 14) | (SEQ ID NO: 18) | (SEQ ID NO: 26) | (SEQ ID NO: 50) | (SEQ ID NO: 90) |
| B03 | NIGMKS | DDSDRPS | QVWDRGMI | GFTFGDRA | ISGSGSGT | ARKRWYGMDV |
| | (SEQ ID NO: 4) | (SEQ ID NO: 13) | (SEQ ID NO: 19) | (SEQ ID NO: 28) | (SEQ ID NO: 55) | (SEQ ID NO: 90) |
| B04 | NIGLKS | DDSDRPS | QVWDRGMI | GFSFSSRA | VSGSGSGT | ARKRWYGMDV |
| | (SEQ ID NO: 5) | (SEQ ID NO: 13) | (SEQ ID NO: 19) | (SEQ ID NO: 31) | (SEQ ID NO: 56) | (SEQ ID NO: 90) |
| B05 | NIGSKM | DDSDRPS | QVWDRGMI | GLTFSSRA | ISGTGATT | ARKRWYGMDV |
| | (SEQ ID NO: 6) | (SEQ ID NO: 13) | (SEQ ID NO: 19) | (SEQ ID NO: 32) | (SEQ ID NO: 49) | (SEQ ID NO: 90) |
| B06 | NIGVKA | DDSDRPS | QVWDRGMI | GFTFSNKA | ISGSGATT | ARKRWYGMDV |
| | (SEQ ID NO: 7) | (SEQ ID NO: 13) | (SEQ ID NO: 19) | (SEQ ID NO: 24) | (SEQ ID NO: 45) | (SEQ ID NO: 90) |
| B07 | NIGSKS | DDSDRPS | QVWDRGMI | GFTFGDRA | ISGSGATT | ARKRWYGMDV |
| | (SEQ ID NO: 1) | (SEQ ID NO: 13) | (SEQ ID NO: 19) | (SEQ ID NO: 28) | (SEQ ID NO: 45) | (SEQ ID NO: 90) |
| B08 | NIGSKS | DDSDRPS | QVWDRGMI | GFTFSDRA | ISASGASR | ARKRWYGMDV |
| | (SEQ ID NO: 1) | (SEQ ID NO: 13) | (SEQ ID NO: 19) | (SEQ ID NO: 30) | (SEQ ID NO: 57) | (SEQ ID NO: 90) |
| B09 | NIGSKS | DDSDRPS | QVWDRGMI | GFTYSSRA | ISSSGTTT | ARKRWYGMDV |
| | (SEQ ID NO: 1) | (SEQ ID NO: 13) | (SEQ ID NO: 19) | (SEQ ID NO: 27) | (SEQ ID NO: 58) | (SEQ ID NO: 90) |
| B10 | NIGSKS | DDSDRPS | QVWGRYQMI | GFTFSTRA | ISGTGDTT | ARKRWYGMDV |
| | (SEQ ID NO: 1) | (SEQ ID NO: 13) | (SEQ ID NO: 20) | (SEQ ID NO: 33) | (SEQ ID NO: 59) | (SEQ ID NO: 90) |
| B11 | NIGSKS | DDSDRPS | QVWDRGMI | GFTFTSRA | ISGSGATA | ARKRWYGMDV |
| | (SEQ ID NO: 1) | (SEQ ID NO: 13) | (SEQ ID NO: 19) | (SEQ ID NO: 25) | (SEQ ID NO: 60) | (SEQ ID NO: 90) |
| B12 | NIGSKS | DDSDRPS | QVWDRGMI | GFTFSDRA | ISGTGAST | ARKRWYGMDV |
| | (SEQ ID NO: 1) | (SEQ ID NO: 13) | (SEQ ID NO: 19) | (SEQ ID NO: 30) | (SEQ ID NO: 61) | (SEQ ID NO: 90) |
| C1 | NIGSKS | DDSDRPS | QVWDRGMI | GFTFDNRA | ISSSGSTM | ARKRWYGMDV |
| | (SEQ ID NO: 1) | (SEQ ID NO: 13) | (SEQ ID NO: 19) | (SEQ ID NO: 29) | (SEQ ID NO: 54) | (SEQ ID NO: 90) |
| C10 | NIGSKS | DDSDRPS | QVWDRGMI | GFTFSSRA | ISASGDTT | ARKRWYGMDV |
| | (SEQ ID NO: 1) | (SEQ ID NO: 13) | (SEQ ID NO: 19) | (SEQ ID NO: 34) | (SEQ ID NO: 62) | (SEQ ID NO: 90) |
| C11 | NIGSKS | DDSDRPS | QVWDRGMI | GFTFSDRA | ISSSASSK | ARKRWYGMDV |
| | (SEQ ID NO: 1) | (SEQ ID NO: 13) | (SEQ ID NO: 19) | (SEQ ID NO: 30) | (SEQ ID NO: 63) | (SEQ ID NO: 90) |
| C12 | NIGSKS | DDSDRPS | QVWDRGMI | GFTFGDRA | ISGTGSTR | ARKRWYGMDV |
| | (SEQ ID NO: 1) | (SEQ ID NO: 13) | (SEQ ID NO: 19) | (SEQ ID NO: 28) | (SEQ ID NO: 64) | (SEQ ID NO: 90) |
| C2 | NIGSKS | DDSDRPS | QVWDRGMI | GFTFTSRA | ISGSGATA | ARKRWYGMDV |
| | (SEQ ID NO: 1) | (SEQ ID NO: 13) | (SEQ ID NO: 19) | (SEQ ID NO: 25) | (SEQ ID NO: 60) | (SEQ ID NO: 90) |
| C3 | NIWGKS | DDIDRPS | QVWDSRTYV | GFTFSSRS | ISGSGSTV | ARKRWYGMDV |
| | (SEQ ID NO: 8) | (SEQ ID NO: 15) | (SEQ ID NO: 18) | (SEQ ID NO: 26) | (SEQ ID NO: 50) | (SEQ ID NO: 90) |
| C4 | NIGSKS | DDSDRPS | QVWDSRTYV | GFTFSSRS | ISGSGATK | ARKRWYGMDV |
| | (SEQ ID NO: 1) | (SEQ ID NO: 13) | (SEQ ID NO: 18) | (SEQ ID NO: 26) | (SEQ ID NO: 65) | (SEQ ID NO: 90) |
| C5 | DIGKKS | DDSDRPS | QVWDSRTYV | GFSFSSRA | ISSSGATT | ARKRWYGMDV |
| | (SEQ ID NO: 9) | (SEQ ID NO: 13) | (SEQ ID NO: 18) | (SEQ ID NO: 31) | (SEQ ID NO: 66) | (SEQ ID NO: 90) |
| C6 | NIGVKS | DDSDRSP | QVWDRGMI | GFTFSRQA | ISYEGRTQ | ARKRWYGMDV |
| | (SEQ ID NO: 3) | (SEQ ID NO: 14) | (SEQ ID NO: 19) | (SEQ ID NO: 35) | (SEQ ID NO: 46) | (SEQ ID NO: 90) |
| C7 | NIGSKS | DDSDRPS | QVWDRGMI | GLTFSRFA | ISSSGTTS | ARKRWYGMDV |
| | (SEQ ID NO: 1) | (SEQ ID NO: 13) | (SEQ ID NO: 19) | (SEQ ID NO: 36) | (SEQ ID NO: 51) | (SEQ ID NO: 90) |
| C8 | NIGSKS | DDSDRPS | QVWDRGMI | GFTFSSRA | ISYEGSTT | ARKRWYGMDV |
| | (SEQ ID NO: 1) | (SEQ ID NO: 13) | (SEQ ID NO: 19) | (SEQ ID NO: 34) | (SEQ ID NO: 67) | (SEQ ID NO: 90) |
| C9 | NIGSKS | DDSDRPS | QVWDRGMI | GFTFSSRS | ISGTGYTT | ARKRWYGMDV |
| | (SEQ ID NO: 1) | (SEQ ID NO: 13) | (SEQ ID NO: 19) | (SEQ ID NO: 26) | (SEQ ID NO: 53) | (SEQ ID NO: 90) |
| D1 | NIGSKS | DDSDRPS | QVWDRGMI | GFTFSSRA | ISSSGTTS | ARKRWYGMDV |
| | (SEQ ID NO: 1) | (SEQ ID NO: 13) | (SEQ ID NO: 19) | (SEQ ID NO: 34) | (SEQ ID NO: 51) | (SEQ ID NO: 90) |
| D10 | NIGSKS | DDSDRPS | QVWDRGMI | GFTFSSRS | ISGSGSTV | ARKRWYGMDV |
| | (SEQ ID NO: 1) | (SEQ ID NO: 13) | (SEQ ID NO: 19) | (SEQ ID NO: 26) | (SEQ ID NO: 50) | (SEQ ID NO: 90) |
| D13 | NIGSKS | DDSDRPS | QVWDRGMI | GFTFSTRA | ISSSGSST | ARKRWYGMDV |
| | (SEQ ID NO: 1) | (SEQ ID NO: 13) | (SEQ ID NO: 19) | (SEQ ID NO: 33) | (SEQ ID NO: 68) | (SEQ ID NO: 90) |
| D12 | NIGSKS | DDSDRPS | QVWASTTAPSV | GFTYSSRA | ISGSGATK | ARKRWYGMDV |
| | (SEQ ID NO: 1) | (SEQ ID NO: 13) | (SEQ ID NO: 21) | (SEQ ID NO: 27) | (SEQ ID NO: 65) | (SEQ ID NO: 90) |
| D2 | NIGSKS | DDSDRPS | QVWDSRTYV | GFSFSTRS | ISGSGATA | ARKRWYGMDV |
| | (SEQ ID NO: 1) | (SEQ ID NO: 13) | (SEQ ID NO: 18) | (SEQ ID NO: 37) | (SEQ ID NO: 60) | (SEQ ID NO: 90) |
| D3 | NIGMKS | DDSDRPS | QVWDRGMI | GFTFSDRA | ISATGAST | ARKRWYGMDV |
| | (SEQ ID NO: 4) | (SEQ ID NO: 13) | (SEQ ID NO: 19) | (SEQ ID NO: 30) | (SEQ ID NO: 69) | (SEQ ID NO: 90) |
| D4 | NIGSKS | DDIDRPA | QVWDRGMI | GFSFSSRA | IDATGRTI | ARKRWYGMDV |
| | (SEQ ID NO: 1) | (SEQ ID NO: 16) | (SEQ ID NO: 19) | (SEQ ID NO: 31) | (SEQ ID NO: 70) | (SEQ ID NO: 90) |
| D5 | NIWGKS | DDIDRPS | QVWDRGMI | GFTFSTRA | ISGTGDTT | ARKRWYGMDV |
| | (SEQ ID NO: 8) | (SEQ ID NO: 15) | (SEQ ID NO: 19) | (SEQ ID NO: 33) | (SEQ ID NO: 59) | (SEQ ID NO: 90) |
| D6 | NIGSKS | DDSDRPS | QVWDSRTYV | GFTFSSRS | ISGTGSTR | ARKRWYGMDV |
| | (SEQ ID NO: 1) | (SEQ ID NO: 13) | (SEQ ID NO: 18) | (SEQ ID NO: 26) | (SEQ ID NO: 64) | (SEQ ID NO: 90) |
| D7 | NIGSKS | DDSDRPS | QVWDRGMI | GFTYSSRA | ISATGTSA | ARKRWYGMDV |
| | (SEQ ID NO: 1) | (SEQ ID NO: 13) | (SEQ ID NO: 19) | (SEQ ID NO: 27) | (SEQ ID NO: 71) | (SEQ ID NO: 90) |
| D8 | NIGSKS | DDSDRPS | QVWDRGMI | GFTFSTRA | ISGSGSNA | ARKRWYGMDV |
| | (SEQ ID NO: 1) | (SEQ ID NO: 13) | (SEQ ID NO: 19) | (SEQ ID NO: 33) | (SEQ ID NO: 48) | (SEQ ID NO: 90) |
| D9 | NIGSKS | DDSDRPS | QVWDRGMI | GFTFSDRA | ISGTAATT | ARKRWYGMDV |
| | (SEQ ID NO: 1) | (SEQ ID NO: 13) | (SEQ ID NO: 19) | (SEQ ID NO: 30) | (SEQ ID NO: 72) | (SEQ ID NO: 90) |
| E1 | NIVTKS | DDIDRPS | QVWDSRTYV | GFTFSSRS | ISGSGSST | ARKRWYGMDV |
| | (SEQ ID NO: 2) | (SEQ ID NO: 15) | (SEQ ID NO: 18) | (SEQ ID NO: 26) | (SEQ ID NO: 73) | (SEQ ID NO: 90) |
| E10 | NIGSKS | DDSDRPS | QVWDRGMI | GFTFSNKA | ISYEGSTK | ARKRWYGMDV |
| | (SEQ ID NO: 1) | (SEQ ID NO: 13) | (SEQ ID NO: 19) | (SEQ ID NO: 24) | (SEQ ID NO: 74) | (SEQ ID NO: 90) |
| E11 | NIGSKS | DDSDRPS | QVWDRGMI | GFTFSSRS | ISSSGTTM | ARKRWYGMDV |
| | (SEQ ID NO: 1) | (SEQ ID NO: 13) | (SEQ ID NO: 19) | (SEQ ID NO: 26) | (SEQ ID NO: 75) | (SEQ ID NO: 90) |
| E12 | NIGSKS | DDSDRPS | QVWDRGMI | GFTFGDRA | ISSSGAIT | ARKRWYGMDV |
| | (SEQ ID NO: 1) | (SEQ ID NO: 13) | (SEQ ID NO: 19) | (SEQ ID NO: 28) | (SEQ ID NO: 66) | (SEQ ID NO: 90) |
| E2 | NIGVKS | DDSDRSP | QVWDRGMI | GFTFSRQA | ISYEGRTQ | ARKRWYGMDV |
| | (SEQ ID NO: 3) | (SEQ ID NO: 14) | (SEQ ID NO: 19) | (SEQ ID NO: 35) | (SEQ ID NO: 46) | (SEQ ID NO: 90) |
| E3 | NIGSKS | DDSDRPS | QVWDRGMI | GFTFSSRA | ISASGDTT | ARKRWYGMDV |
| | (SEQ ID NO: 1) | (SEQ ID NO: 13) | (SEQ ID NO: 19) | (SEQ ID NO: 34) | (SEQ ID NO: 62) | (SEQ ID NO: 90) |
| E4 | DIGKKS | DDSDRPS | QVWDRGMI | GFTFSDRA | ISSSATTM | ARKRWYGMDV |
| | (SEQ ID NO: 9) | (SEQ ID NO: 13) | (SEQ ID NO: 19) | (SEQ ID NO: 30) | (SEQ ID NO: 76) | (SEQ ID NO: 90) |
| E5 | NIGSKS | DDSDRPS | QVWDRGMI | GFTFGDKA | ISSSGTTM | ARKRWYGMDV |
| | (SEQ ID NO: 1) | (SEQ ID NO: 13) | (SEQ ID NO: 19) | (SEQ ID NO: 38) | (SEQ ID NO: 75) | (SEQ ID NO: 90) |
| E6 | NIWGKS | DDIDRPS | QVWDRGMI | GFTFSSRS | ISGSGATK | ARKRWYGMDV |
| | (SEQ ID NO: 8) | (SEQ ID NO: 15) | (SEQ ID NO: 19) | (SEQ ID NO: 26) | (SEQ ID NO: 65) | (SEQ ID NO: 90) |
| E7 | NIGSKS | DDSDRPS | QVWDRGMI | GFTFGDKA | ISSSGTTM | ARKRWYGMDV |
| | (SEQ ID NO: 1) | (SEQ ID NO: 13) | (SEQ ID NO: 19) | (SEQ ID NO: 38) | (SEQ ID NO: 75) | (SEQ ID NO: 90) |
| E8 | NIGSKS | DDSDRPS | QVWDRGMI | GFTFSRFA | ISSSGSTA | ARKRWYGMDV |
| | (SEQ ID NO: 1) | (SEQ ID NO: 13) | (SEQ ID NO: 19) | (SEQ ID NO: 39) | (SEQ ID NO: 76) | (SEQ ID NO: 90) |
| E9 | NIGSKS | DDSDRPS | QVWDRGMI | GFTFSTKA | ISSSGATT | ARKRWYGMDV |
| | (SEQ ID NO: 1) | (SEQ ID NO: 13) | (SEQ ID NO: 19) | (SEQ ID NO: 40) | (SEQ ID NO: 66) | (SEQ ID NO: 90) |
| F1 | NIGSKS | DDSDRPS | QVWDRGMI | GFTFSSRA | ISGSGSTM | ARKRWYGMDV |
| | (SEQ ID NO: 1) | (SEQ ID NO: 13) | (SEQ ID NO: 19) | (SEQ ID NO: 34) | (SEQ ID NO: 77) | (SEQ ID NO: 90) |
| F10 | NIGSKS | DDSDRPS | QVWDRGMI | GFTFSSRA | ISGSGATA | ARKRWYGMDV |
| | (SEQ ID NO: 1) | (SEQ ID NO: 13) | (SEQ ID NO: 19) | (SEQ ID NO: 34) | (SEQ ID NO: 60) | (SEQ ID NO: 90) |
| F11 | NIGSKS | DDSDRPS | QVWDRGMI | GFTFSDRA | ISGSGFTT | ARKRWYGMDV |
| | (SEQ ID NO: 1) | (SEQ ID NO: 13) | (SEQ ID NO: 19) | (SEQ ID NO: 30) | (SEQ ID NO: 78) | (SEQ ID NO: 90) |
| F12 | NIGSKS | DDSDRPS | QVWDRGMI | GFTFGDKA | ISSSGTTM | ARKRWYGMDV |
| | (SEQ ID NO: 1) | (SEQ ID NO: 13) | (SEQ ID NO: 19) | (SEQ ID NO: 38) | (SEQ ID NO: 75) | (SEQ ID NO: 90) |
| F2 | NIWGKS | DDIDRPS | QVWDSRTYV | GFTFSSRS | ISGRGDVT | ARKRWYGMDV |
| | (SEQ ID NO: 8) | (SEQ ID NO: 15) | (SEQ ID NO: 18) | (SEQ ID NO: 26) | (SEQ ID NO: 79) | (SEQ ID NO: 90) |
| F3 | NIGSKS | DDSDRPS | QVWDRGMI | GFTFDNRA | ISYEGRTQ | ARKRWYGMDV |
| | (SEQ ID NO: 1) | (SEQ ID NO: 13) | (SEQ ID NO: 19) | (SEQ ID NO: 29) | (SEQ ID NO: 46) | (SEQ ID NO: 90) |
| F4 | NIGSKS | DDSDRPS | QVWDRGMI | GFTFSSRS | ISGSGDTM | ARKRWYGMDV |
| | (SEQ ID NO: 1) | (SEQ ID NO: 13) | (SEQ ID NO: 19) | (SEQ ID NO: 26) | (SEQ ID NO: 80) | (SEQ ID NO: 90) |
| F5 | NIGSKS | DDSDRPS | QVWDRGMI | GFTFTSRA | ISGSGATA | ARKRWYGMDV |
| | (SEQ ID NO: 1) | (SEQ ID NO: 13) | (SEQ ID NO: 19) | (SEQ ID NO: 25) | (SEQ ID NO: 60) | (SEQ ID NO: 90) |
| F6 | NIGLKS | DDSDRPS | QVWDRGMI | GLTFSIYA | ISGSGATA | ARKRWYGMDV |
| | (SEQ ID NO: 5) | (SEQ ID NO: 13) | (SEQ ID NO: 19) | (SEQ ID NO: 41) | (SEQ ID NO: 60) | (SEQ ID NO: 90) |
| F7 | NIGSKS | DDSDRPS | QVWDRGMI | GLTFSSRA | ISSSGTTT | ARKRWYGMDV |
| | (SEQ ID NO: 1) | (SEQ ID NO: 13) | (SEQ ID NO: 19) | (SEQ ID NO: 32) | (SEQ ID NO: 58) | (SEQ ID NO: 90) |
| F8 | NIGSKS | DDSDRPS | QVWDSRSVV | GFTFSSRA | ISSSGSTA | ARKRWYGMDV |
| | (SEQ ID NO: 1) | (SEQ ID NO: 13) | (SEQ ID NO: 22) | (SEQ ID NO: 34) | (SEQ ID NO: 76) | (SEQ ID NO: 90) |
| F9 | NIGSKS | DDSDRPS | QVWDRGMI | GFTFSDRA | ISGSGKST | ARKRWYGMDV |
| | (SEQ ID NO: 1) | (SEQ ID NO: 13) | (SEQ ID NO: 19) | (SEQ ID NO: 30) | (SEQ ID NO: 81) | (SEQ ID NO: 90) |
| G1 | NIGMKS | DDSDRPS | QVWDRGMI | GFIFSTKA | ISGSGATA | ARKRWYGMDV |
| | (SEQ ID NO: 4) | (SEQ ID NO: 13) | (SEQ ID NO: 19) | (SEQ ID NO: 40) | (SEQ ID NO: 60) | (SEQ ID NO: 90) |
| G10 | NIGSKS | DDSDRPS | QVWDRGMI | GFTFGDRA | ISGTGRST | ARKRWYGMDV |
| | (SEQ ID NO: 1) | (SEQ ID NO: 13) | (SEQ ID NO: 19) | (SEQ ID NO: 28) | (SEQ ID NO: 82) | (SEQ ID NO: 90) |
| G11 | NIGSKS | DDSDRPS | QVWDRGMI | GFTFSSRA | ISSSGTTM | ARKRWYGMDV |
| | (SEQ ID NO: 1) | (SEQ ID NO: 13) | (SEQ ID NO: 19) | (SEQ ID NO: 34) | (SEQ ID NO: 75) | (SEQ ID NO: 90) |
| G12 | NIGSKS | DDSDRPS | QVWDRGMI | GFTFSSRS | ISASGTTI | ARKRWYGMDV |
| | (SEQ ID NO: 1) | (SEQ ID NO: 13) | (SEQ ID NO: 19) | (SEQ ID NO: 26) | (SEQ ID NO: 83) | (SEQ ID NO: 90) |
| G2 | NIGMKS | DDSDRPS | QVWDRGMI | GFTFDNRA | ISGSGDTK | ARKRWYGMDV |
| | (SEQ ID NO: 4) | (SEQ ID NO: 13) | (SEQ ID NO: 19) | (SEQ ID NO: 29) | (SEQ ID NO: 84) | (SEQ ID NO: 90) |
| G3 | NIGYKS | DDSDRPS | QVWDRGMI | GFTFTSRA | ISASGDTT | ARKRWYGMDV |
| | (SEQ ID NO: 10) | (SEQ ID NO: 13) | (SEQ ID NO: 19) | (SEQ ID NO: 25) | (SEQ ID NO: 62) | (SEQ ID NO: 90) |
| G4 | NIVTKS | DDSDRPS | QVWDRGMI | AFTFSRFA | ISSSGSTA | ARKRWYGMDV |
| | (SEQ ID NO: 2) | (SEQ ID NO: 13) | (SEQ ID NO: 19) | (SEQ ID NO: 42) | (SEQ ID NO: 76) | (SEQ ID NO: 90) |
| G5 | KLGDKW | EDVDRPS | QVWDSRTYV | GFTFSSRS | ISGSGSTV | ARKRWYGMDV |
| | (SEQ ID NO: 11) | (SEQ ID NO: 17) | (SEQ ID NO: 18) | (SEQ ID NO: 26) | (SEQ ID NO: 50) | (SEQ ID NO: 90) |
| G6 | NIGLKS | DDSDRPS | QVWDRGMI | GFTFDNRA | ISSTGKST | ARKRWYGMDV |
| | (SEQ ID NO: 5) | (SEQ ID NO: 13) | (SEQ ID NO: 19) | (SEQ ID NO: 29) | (SEQ ID NO: 85) | (SEQ ID NO: 90) |
| G7 | NIGSKS | DDSDRPS | QVWDRGMI | GFIFSTKA | ISYEGRTQ | ARKRWYGMDV |
| | (SEQ ID NO: 1) | (SEQ ID NO: 13) | (SEQ ID NO: 19) | (SEQ ID NO: 40) | (SEQ ID NO: 46) | (SEQ ID NO: 90) |
| G8 | NIGSKS | DDSDRPS | QVWDRGMI | GFTYSSRA | ISASGDTT | ARKRWYGMDV |
| | (SEQ ID NO: 1) | (SEQ ID NO: 13) | (SEQ ID NO: 19) | (SEQ ID NO: 27) | (SEQ ID NO: 62) | (SEQ ID NO: 90) |
| G9 | NIGSKS | DDSDRPS | QVWDRGMI | GFTFSDRA | ISASAATT | ARKRWYGMDV |
| | (SEQ ID NO: 1) | (SEQ ID NO: 13) | (SEQ ID NO: 19) | (SEQ ID NO: 30) | (SEQ ID NO: 86) | (SEQ ID NO: 90) |
| H1 | NIGVKS | DDSDRSP | QVWDRGMI | GFTFSRQA | ISYEGRTQ | ARKRWYGMDV |
| | (SEQ ID NO: 3) | (SEQ ID NO: 14) | (SEQ ID NO: 19) | (SEQ ID NO: 35) | (SEQ ID NO: 46) | (SEQ ID NO: 90) |
| H10 | NIGSKS | DDSDRPS | QVWDRGMI | GFTFSSRS | ISGSGDTT | ARKRWYGMDV |
| | (SEQ ID NO: 1) | (SEQ ID NO: 13) | (SEQ ID NO: 19) | (SEQ ID NO: 26) | (SEQ ID NO: 47) | (SEQ ID NO: 90) |
| H11 | NIGSKS | DDSDRPS | QVWDRGMI | GFTFTSRA | ISASGDTT | ARKRWYGMDV |
| | (SEQ ID NO: 1) | (SEQ ID NO: 13) | (SEQ ID NO: 19) | (SEQ ID NO: 25) | (SEQ ID NO: 62) | (SEQ ID NO: 90) |
| H2 | NIGYKS | DDSDRPS | QVWDSRTYV | GFTFSSRA | ISASGDTT | ARKRWYGMDV |
| | (SEQ ID NO: 10) | (SEQ ID NO: 13) | (SEQ ID NO: 18) | (SEQ ID NO: 34) | (SEQ ID NO: 62) | (SEQ ID NO: 90) |
| H3 | NIGSKS | DDSDRPS | QVWDRGMI | GFTFSSRA | ISSSGTTT | ARKRWYGMDV |
| | (SEQ ID NO: 1) | (SEQ ID NO: 13) | (SEQ ID NO: 19) | (SEQ ID NO: 34) | (SEQ ID NO: 58) | (SEQ ID NO: 90) |
| H5 | NIGFKS | DDSDRPS | QVWDRGMI | GLIFSNFA | ISSSGTTM | ARKRWYGMDV |
| | (SEQ ID NO: 12) | (SEQ ID NO: 13) | (SEQ ID NO: 19) | (SEQ ID NO: 43) | (SEQ ID NO: 75) | (SEQ ID NO: 90) |
| H7 | NIGSKS | DDSDRPS | QVWDRGMI | GFTFSDRA | ISGSGFTT | ARKRWYGMDV |
| | (SEQ ID NO: 1) | (SEQ ID NO: 13) | (SEQ ID NO: 19) | (SEQ ID NO: 30) | (SEQ ID NO: 78) | (SEQ ID NO: 90) |
| H8 | NIGSKS | DDSDRPS | QVWDRGMI | GFTFSSRA | ISGPGMTS | ARKRWYGMDV |
| | (SEQ ID NO: 1) | (SEQ ID NO: 13) | (SEQ ID NO: 19) | (SEQ ID NO: 34) | (SEQ ID NO: 87) | (SEQ ID NO: 90) |
| H9 | NIGSKS | DDSDRPS | QVWDRGMI | GFTFSDRA | ISGSGTTK | ARKRWYGMDV |
| | (SEQ ID NO: 1) | (SEQ ID NO: 13) | (SEQ ID NO: 19) | (SEQ ID NO: 30) | (SEQ ID NO: 88) | (SEQ ID NO: 90) |

TABLE 5
Light chain variable region and heavy chain variable region sequences.

| Clone | Light chain | Heavy chain |
|---|---|---|
| Parental | SYELTQPPSVSVAPGKTARITCGGNNIGSKSVHWYQQKPGQA | EVQLVESGGGVVQPGRSLRLSCAASGFTFSSYSMNWVRQAPGKGL |
| (H6.1) | PVLVVYDDSDRPSGIPERFSGSNSGNTATLTISRVEAGDEAD | EWVSAISGSGGSTYYADSVKGRFTISRDNSKNTLYLQMNSLRAED |
| | YYCQVWDSRTYVFGTGTKLTVL (SEQ ID NO: 96) | TAVYYCARKRWYGMDVWGQGTTVTVSS (SEQ ID NO: 119) |
| A01 | SYELTQPPSVSVAPGKTARITCGGNNIGSKSVHWYQQKPGQA | EVQLVESGGGVVQPGRSLRLSCAASGFTFSNKAMNWVRQAPGKGL |
| | PVLVVYDDSDRPSGIPERFSGSNSGNTATLTISRVEAGDEAD | EWVSAISGSGATTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 120) |
| A02 | SYELTQPPSVSVAPGKTARITCGGNNIGSKSVHWYQQKPGQA | EVQLVESGGGVVQPGRSLRLSCAASGFTFTSRAMNWVRQAPGKGL |
| | PVLVVYDDSDRPSGIPERFSGSNSGNTATLTISRVEAGDEAD | EWVSAISYEGRTQYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 121) |
| A04 | SYELTQPPSVSVAPGKTARITCGGNNIGSKSVHWYQQKPGQA | EVQLVESGGGVVQPGRSLRLSCAASGFTFSSRSMNWVRQAPGKGL |
| | PVLVVYDDSDRPSGIPERFSGSNSGNTATLTISRVEAGDEAD | EWVSAISGSGDTTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 122) |
| A05 | SYELTQPPSVSVAPGKTARITCGGNNIGSKSVHWYQQKPGQA | EVQLVESGGGVVQPGRSLRLSCAASGFTYSSRAMNWVRQAPGKGL |
| | PVLVVYDDSDRPSGIPERFSGSNSGNTATLTISRVEAGDEAD | EWVSAISGSGSNAYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 123) |
| A06 | SYELTQPPSVSVAPGKTARITCGGNNIVTKSVHWYQQKPGQA | EVQLVESGGGVVQPGRSLRLSCAASGFTFGDRAMNWVRQAPGKGL |
| | PVLVVYDDSDRPSGIPERFSGSNSGNTATLTISRVEAGDEAD | EWVSAISGTGATTYYADSVKGRFTISRDNSKNTLYLQMNSLRAED |
| | YYCQVWDRGMIFGTGTKLTVL (SEQ ID NO: 98) | TAVYYCARKRWYGMDVWGQGTTVTVSS (SEQ ID NO: 124) |
| A07 | SYELTQPPSVSVAPGKTARITCGGNNIGSKSVHWYQQKPGQA | EVQLVESGGGVVQPGRSLRLSCAASGFTFSSRSMNWVRQAPGKGL |
| | PVLVVYDDSDRPSGIPERFSGSNSGNTATLTISRVEAGDEAD | EWVSAISGSGSTVYYADSVKGRFTISRDNSKNTLYLQMNSLRAED |
| | YYCQVWDSRTYVFGTGTKLTVL (SEQ ID NO: 96) | TAVYYCARKRWYGMDVWGQGTTVTVSS (SEQ ID NO: 125) |
| A08 | SYELTQPPSVSVAPGKTARITCGGNNIGSKSVHWYQQKPGQA | EVQLVESGGGVVQPGRSLRLSCAASGFTFGDRAMNWVRQAPGKGL |
| | PVLVVYDDSDRPSGIPERFSGSNSGNTATLTISRVEAGDEAD | EWVSAISSSGTTSYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 126) |
| A09 | SYELTQPPSVSVAPGKTARITCGGNNIGSKSVHWYQQKPGQA | EVQLVESGGGVVQPGRSLRLSCAASGFTFSSRSMNWVRQAPGKGL |
| | PVLVVYDDSDRPSGIPERFSGSNSGNTATLTISRVEAGDEAD | EWVSAISGSGLTSYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 127) |
| A10 | SYELTQPPSVSVAPGKTARITCGGNNIGSKSVHWYQQKPGQA | EVQLVESGGGVVQPGRSLRLSCAASGFTFSSRSMNWVRQAPGKGL |
| | PVLVVYDDSDRESGIPERFSGSNSGNTATLTISRVEAGDEAD | EWVSAISGIGYTTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 128) |
| A11 | SYELTQPPSVSVAPGKTARITCGGNNIGSKSVHWYQQKPGQA | EVQLVESGGGVVQPGRSLRLSCAASGFTFSSRSMNWVRQAPGKGL |
| | PVLVVYDDSDRPSGIPERFSGSNSGNTATLTISRVEAGDEAD | EWVSAISGSGSTVYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 125) |
| A12 | SYELTQPPSVSVAPGKTARITCGGNNIGSKSVHWYQQKPGQA | EVQLVESGGGVVQPGRSLRLSCAASGFTFDNRAMNWVRQAPGKGL |
| | PVLVVYDDSDRPSGIPERFSGSNSGNTATLTISRVEAGDEAD | EWVSAISSSGSTMYYADSVKGRFTISRDNSKNTLYLQMNSLRAED |
| | YYCQVWDSRTYVFGTGTKLTVL (SEQ ID NO: 96) | TAVYYCARKRWYGMDVWGQGTTVTVSS (SEQ ID NO: 129) |
| B01 | SYELTQPPSVSVAPGKTARITCGGNNIGVKSVHWYQQKPGQA | EVQLVESGGGVVQPGRSLRLSCAASGFTFSDRAMNWVRQAPGKGL |
| | PVLVVYDDSDRPSGIPERFSGSNSGNTATLTISRVEAGDEAD | EWVSAISYEGRTQYYADSVKGRFTISRDNSKNTLYLQMNSLRAED |
| | YYCQVWDSRTYVFGTGTKLTVL (SEQ ID NQ; 99) | TAVYYCARKRWYGMDVWGQGTTVTVSS (SEQ ID NO: 130) |
| B02 | SYELTQPPSVSVAPGKTARITCGGNNIGVKSVHWYQQKPGQA | EVQLVESGGGVVQPGRSLRLSCAASGFTFSSRSMNWVRQAPGKGL |
| | PVLVVYDDSDRSPGIPERFSGSNSGNTATLTISRVEAGDEAD | EWVSAISGSGSTVYYADSVKGRFTISRDNSKNTLYLQMNSLRAED |
| | YYCQVWDSRTYVFGTGTKLTVL (SEQ ID NO: 100) | TAVYYCARKRWYGMDVWGQGTTVTVSS (SEQ ID NO: 125) |
| B03 | SYELTQPPSVSVAPGKTARITCGGNNIGMKSVHWYQQKPGQA | EVQLVESGGGVVQPGRSLRLSCAASGFTFGDRAMNWVRQAPGKGL |
| | PVLVVYDDSDRPSGIPERPSGSNSGNTATLTISRVEAGDEAD | EWVSAISGSGSGTYYADSVKGRFTISRDNSKNTLYLQMNSLRAED |
| | YYCQVWDRGMIFGTGTKLTVL (SEQ ID NO: 101) | TAVYYCARKRWYGMDVWGQGTTVTVSS (SEQ ID NO: 131) |
| B04 | SYELTQPPSVSVAPGKTARITCGGNNIGLKSVHWYQQKPGQA | EVQLVESGGGVVQPGRSLRLSCAASGFSFSSRAMNWVRQAPGKGL |
| | PVLVVYDDSDRPSGIPERFSGSNSGNTATLTISRVEAGDEAD | EWVSAVSGSGSGTYYADSVKGRFTISRDNSKNTLYLQMNSLRAED |
| | YYCQVWDRGMIFGTGTKLTVL (SEQ ID NO: 102) | TAVYYCARKRWYGMDVWGQGTTVTVSS (SEQ ID NO: 132) |
| B05 | SYELTQPPSVSVAPGKTARITCGGNNIGSKMVHWYQQKPGQA | EVQLVESGGGVVQPGRSLRLSCAASGLTFSSRAMNWVRQAPGKGL |
| | PVLVVYDDSDRPSGIPERFSGSNSGNTATLTISRVEAGDEAD | EWVSAISGTGATTYYADSVKGRFTISRDNSKNTLYLQMNSLRAED |
| | YYCQVWDRGMIFGTGTKLTVL (SEQ ID NO: 103) | TAVYYCARKRWYGMDVWGQGTTVTVSS (SEQ ID NO: 133) |
| B06 | SYELTQPPSVSVAPGKTARITCGGNNIGVKAVHWYQQKPGQA | EVQLVESGGGVVQPGRSLRLSCAASGFTFSNKAMNWVRQAPGKGL |
| | PVLVVYDDSDRPSGIPERFSGSNSGNTATLTISRVEAGDEAD | EWVSAISGSGATTYYADSVKGRFTISRDNSKNTLYLQMNSLRAED |
| | YYCQVWDRGMIFGTGTKLTVL (SEQ ID NO: 104) | TAVYYCARKRWYGMDVWGQGTTVTVSS (SEQ ID NO: 120) |
| B07 | SYELTQPPSVSVAPGKTARITCGGNNIGSKSVHWYQQKPGQA | EVQLVESGGGVVQPGRSLRLSCAASGFTFGDRAMNWVRQAPGKGL |
| | PVLVVYDDSDRPSGIPERFSGSNSGNTATLTISRVEAGDEAD | EWVSAISGSGATTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 134) |
| B08 | SYELTQPPSVSVAPGKTARITCGGNNIGSKSVHWYQQKPGQA | EVQLVESGGGVVQPGRSLRLSCAASGFTFSDRAMNWVRQAPGKGL |
| | PVLVVYDDSDRPSGIPERFSGSNSGNTATLTISRVEAGDEAD | EWVSAISASGASRYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 135) |
| B09 | SYELTQPPSVSVAPGKTARITCGGNNIGSKSVHWYQQKPGQA | EVQLVESGGGVVQPGRSLRLSCAASGFTYSSRAMNWVRQAPGKGL |
| | PVLVVYDDSDRPSGIPERFSGSNSGNTATLTISRVEAGDEAD | EWVSAISSSGTTTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 136) |
| B10 | SYELTQPPSVSVAPGKTARITCGGNNIGSKSVHWYQQKPGQA | EVQLVESGGGVVQPGRSLRLSCAASGFTFSTRAMNWVRQAPGKGL |
| | PVLVVYDDSDRPSGIPERFSGSNSGNTATLTISRVEAGDEAD | EWVSAISGTGDTTYYADSVKGRFTISRDNSKNTLYLQMNSLRAED |
| | YYCQVWGRYQMIFGTGTKLTVL (SEQ ID NO: 105) | TAVYYCARKRWYGMDVWGQGTTVTVSS (SEQ ID NO: 137) |
| B11 | SYELTQPPSVSVAPGKTARITCGGNNIGSKSVHWYQQKPGQA | EVQLVESGGGVVQPGRSLRLSCAASGFTFTSRAMNWVRQAPGKGL |
| | PVLVVYDDSDRPSGIPERFSGSNSGNTATLTISRVEAGDEAD | EWVSAISGSGATAYYADSVKGRFTISRDNSKNTLYLQMNSLRAED |
| | YYCQVWDRGMIFGTGTKLIVL (SEQ ID NO: 97) | TAVYYCARKRWYGMDVWGQGTTVTVSS (SEQ ID NO: 138) |
| B12 | SYELTQPPSVSVAPGKTARITCGGNNIGSKSVHWYQQKPGQA | EVQLVESGGGVVQPGRSLRLSCAASGFTFSDRAMNWVRQAPGKGL |
| | PVLVVYDDSDRPSGIPERFSGSNSGNTATLTISRVEAGDEAD | EWVSAISGTGASTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 139) |
| C01 | SYELTQPPSVSVAPGKTARITCGGNNIGSKSVHWYQQKPGQA | EVQLVESGGGVVQPGRSLRLSCAASGFTFDNRAMNWVRQAPGKGL |
| | PVLVVYDDSDRPSGIPERFSGSNSGNTATLTISRVEAGDEAD | EWVSAISSSGSTMYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 129) |
| C03 | SYELTQPPSVSVAPGKTARITCGGNNIWGKSVHWYQQKPGQA | EVQLVESGGGVVQPGRSLRLSCAASGFTFSSRSMNWVRQAPGKGL |
| | PVLVVYDDIDRPSGIPERFSGSNSGNTATLTISRVEAGDEAD | EWVSAISGSGSTVYYADSVKGRFTISRDNSKNTLYLQMNSLRAED |
| | YYCQVWDSRTYVFGTGTKLTVL (SEQ ID NO: 106) | TAVYYCARKRWYGMDVWGQGTTVTVSS (SEQ ID NO: 125) |
| C04 | SYELTQPPSVSVAPGKTARITCGGNNIGSKSVHWYQQKPGQA | EVQLVESGGGVVQPGRSLRLSCAASGFTFSSRSMNWVRQAPGKGL |
| | PVLVVYDDSDRESGIPERFSGSNSGNTATLTISRVEAGDEAD | EWVSAISGSGATKYYADSVKGRFTISRDNSKNTLYLQMNSLRAED |
| | YYCQVWDSRTYVFGTGTKLTVL (SEQ ID NO: 96) | TAVYYCARKRWYGMDVWGQGTTVTVSS (SEQ ID NO: 140) |
| C05 | SYELTQPPSVSVAPGKTARITCGGNDIGKKSVHWYQQKPGQA | EVQLVESGGGVVQPGRSLRLSCAASGFSFSSRAMNWVRQAPGKGL |
| | PVLVVYDDSDRPSGIPERFSGSNSGNTATLTISRVEAGDEAD | EWVSAISSSGATTYYADSVKGRFTISRDNSKNTLYLQMNSLRAED |
| | YYCQVWDSRTYVFGTGTKLTVL (SEQ ID NO: 107) | TAVYYCARKRWYGMDVWGQGTTVTVSS ( SEQ ID NO: 141) |
| C06 | SYELTQPPSVSVAPGKTARITCGGNNIGVKSVHWYQQKPGQA | EVQLVESGGGVVQPGRSLRLSCAASGFTFSRQAMNWVRQAPGKGL |
| | PVLVVYDDSDRSPGIPERFSGSNSGNTATLTISRVEAGDEAD | EWVSAISYEGRTQYYADSVKGRFTISRDNSKNTLYLQMNSLRAED |
| | YYCQVWDRGMIFGTGTKLTVL (SEQ ID NO: 108) | TAVYYCARKRWYGMDVWGQGTTVTVSS (SEQ ID NO: 142) |
| C07 | SYELTQPPSVSVAPGKTARITCGGNNIGSKSVHWYQQKPGQA | EVQLVESGGGVVQPGRSLRLSCAASGLTFSRFAMNWVRQAPGKGL |
| | PVLVVYDDSDRPSGIPERFSGSNSGNTATLTISRVEAGDEAD | EWVSAISSSGTTSYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 143) |
| C08 | SYELTQPPSVSVAPGKTARITCGGNNIGSKSVHWYQQKPGQA | EVQLVESGGGVVQPGRSLRLSCAASGFTFSSRAMNWVRQAPGKGL |
| | PVLVVYDDSDRPSGIPERFSGSNSGNTATLTISRVEAGDEAD | EWVSAISYEGSTTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 144) |
| C10 | SYELTQPPSVSVAPGKTARITCGGNNIGSKSVHWYQQKPGQA | EVQLVESGGGVVQPGRSLRLSCAASGFTFSSRAMNWVRQAPGKGL |
| | PVLVVYDDSDRPSGIPERFSGSNSGNTATLTISRVEAGDEAD | EWVSAISASGDTTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 145) |
| C11 | SYELTQPPSVSVAPGKTARITCGGNNIGSKSVHWYQQKPGQA | EVQLVESGGGVVQPGRSLRLSCAASGFTFSDRAMNWVRQAPGKGL |
| | PVLVVYDDSDRPSGIPERFSGSNSGNTATLTISRVEAGDEAD | EWVSAISSSASSKYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 146) |
| C12 | SYELTQPPSVSVAPGKTARITCGGNNIGSKSVHWYQQKPGQA | EVQLVESGGGVVQPGRSLRLSCAASGFTFGDRAMNWVRQAPGKGL |
| | PVLVVYDDSDRPSGIPERFSGSNSGNTATLTISRVEAGDEAD | EWVSAISGTGSTRYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 147) |
| D01 | SYELTQPPSVSVAPGKTARITCGGNNIGSKSVHWYQQKPGQA | EVQLVESGGGVVQPGRSLRLSCAASGFTFSSRAMNWVRQAPGKGL |
| | PVLVVYDDSDRPSGIPERFSGSNSGNTATLTISRVEAGDEAD | EWVSAISSSGTTSYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 148) |
| D02 | SYELTQPPSVSVAPGKTARITCGGNNIGSKSVHWYQQKPGQA | EVQLVESGGGVVQPGRSLRLSCAASGFSFSTRSMNWVRQAPGKGL |
| | PVLVVYDDSDRPSGIPERFSGSNSGNTATLTISRVEAGDEAD | EWVSAISGSGATAYYADSVKGRFTISRDNSKNTLYLQMNSLRAED |
| | YYCQVWDSRTYVFGTGTKLTVL (SEQ ID NO: 96) | TAVYYCARKRWYGMDVWGQGTTVTVSS (SEQ ID NO: 149) |
| D03 | SYELTQPPSVSVAPGKTARITCGGNNIGMKSVHWYQQKPGQA | EVQLVESGGGVVQPGRSLRLSCAASGFTFSDRAMNWVRQAPGKGL |
| | PVLVVYDDSDRPSGIPERFSGSNSGNTATLTISRVEAGDEAD | EWVSAISATGASTYYADSVKGRFTISRDNSKNTLYLQMNSLRAED |
| | YYCQVWDRGMIFGTGTKLTVL (SEQ ID NO: 101) | TAVYYCARKRWYGMDVWGQGTTVTVSS (SEQ ID NO: 150) |
| D04 | SYELTQPPSVSVAPGKTARITCGGNNIGSKSVHIWYQQKPGQA | EVQLVESGGGVVQPGRSLRLSCAASGFSFSSRAMNWVRQAPGKGL |
| | PVLVVYDDIDRPAGIPERFSGSNSGNTATLTISRVEAGDEAD | EWVSAIDATGRTIYYADSVKGRFTISRDNSKNTLYLQMNSLRAED |
| | YYCQVWDRGMIFGTGTKLTVL (SEQ ID NO: 109) | TAVYYCARKRWYGMDVWGQGTTVTVSS (SEQ ID NO: 151) |
| D05 | SYELTQPPSVSVAPGKTARITCGGNNIWGKSVHWYQQKPGQA | EVQLVESGGGVVQPGRSLRLSCAASGFTFSTRAMNWVRQAPGKGL |
| | PVLVVYDDIDRPSGIPERFSGSNSGNTATLTISRVEAGDEAD | EWVSAISGTGDTTYYADSVKGRFTISRDNSKNTLYLQMNSLRAED |
| | YYCQVWDRGMIFGTGTKLTVL (SEQ ID NO: 110) | TAVYYCARKRWYGMDVWGQGTTVTVSS (SEQ ID NO: 137) |
| D06 | SYELTQPPSVSVAPGKTARITCGGNNIGSKSVHWYQQKPGQA | EVQLVESGGGVVQPGRSLRLSCAASGFTFSSRSMNWVRQAPGKGL |
| | PVLVVYDDSDRESGIPERFSGSNSGNTATLTISRVEAGDEAD | EWVSAISGIGSTRYYADSVKGRFTISRDNSKNTLYLQMNSLRAED |
| | YYCQVWDSRTYVFGTGTKLTVL (SEQ ID NO: 96) | TAVYYCARKRWYGMDVWGQGTTVTVSS (SEQ ID NO: 152) |
| D07 | SYELTQPPSVSVAPGKTARITCGGNNIGSKSVHWYQQKPGQA | EVQLVESGGGVVQPGRSLRLSCAASGFTYSSRAMNWVRQAPGKGL |
| | PVLVVYDDSDRPSGIPERFSGSNSGNTATLTISRVEAGDEAD | EWVSAISATGTSAYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 153) |
| D08 | SYELTQPPSVSVAPGKTARITCGGNNIGSKSVHWYQQKPGQA | EVQLVESGGGVVQPGRSLRLSCAASGFTFSTRAMNWVRQAPGKGL |
| | PVLVVYDDSDRPSGIPERFSGSNSGNTATLTISRVEAGDEAD | EWVSAISGSGSNAYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 154) |
| D09 | SYELTQPPSVSVAPGKTARITCGGNNIGSKSVHWYQQKPGQA | EVQLVESGGGVVQPGRSLRLSCAASGFTFSDRAMNWVRQAPGKGL |
| | PVLVVYDDSDRPSGIPERFSGSNSGNTATLTISRVEAGDEAD | EWVSAISGTAATTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 155) |
| D11 | SYELTQPPSVSVAPGKTARITCGGNNIGSKSVHWYQQKPGQA | EVQLVESGGGVVQPGRSLRLSCAASGFTFSTRAMNWVRQAPGKGL |
| | PVLVVYDDSDRPSGIPERFSGSNSGNTATLTISRVEAGDEAD | EWVSAISSSGSSTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 156) |
| D12 | SYELTQPPSVSVAPGKTARITCGGNNIGSKSVHWYQQKPGQA | EVQLVESGGGVVQPGRSLRLSCAASGFTYSSRAMNWVRQAPGKGL |
| | PVLVVYDDSDRPSGIPERFSGSNSGNTATLTISRVEAGDEAD | EWVSAISGSGATKYYADSVKGRFTISRDNSKNTLYLQMNSLRAED |
| | YYCQVWASTTAPSVFGTGTKLTVL (SEQ ID NO: 111) | TAVYYCARKRWYGMDVWGQGTTVTVSS (SEQ ID NO: 157) |
| E01 | SYELTQPPSVSVAPGKTARITCGGNNIVTKSVHWYQQKPGQA | EVQLVESGGGVVQPGRSLRLSCAASGFTFSSRSMNWVRQAPGKGL |
| | PVLVVYDDIDRPSGIPERFSGSNSGNTATLTISRVEAGDEAD | EWVSAISGSGSSTYYADSVKGRFTISRDNSKNTLYLQMNSLRAED |
| | YYCQVWDSRTYVFGTGTKLTVL (SEQ ID NO: 112) | TAVYYCARKRWYGMDVWGQGTTVTVSS (SEQ ID NO: 158) |
| E04 | SYELTQPPSVSVAPGKTARITCGGNDIGKKSVHWYQQKPGQA | EVQLVESGGGVVQPGRSLRLSCAASGFTFSDRAMNWVRQAPGKGL |
| | PVLVVYDDSDRESGIPERFSGSNSGNTATLTISRVEAGDEAD | EWVSAISSSATTMYYADSVKGRFTISRDNSKNTLYLQMNSLRAED |
| | YYCQVWDRGMIFGTGTKLTVL (SEQ ID NO: 113) | TAVYYCARKRWYGMDVWGQGTTVTVSS (SEQ ID NO: 159) |
| E05 | SYELTQPPSVSVAPGKTARITCGGNNIGSKSVHWYQQKPGQA | EVQLVESGGGVVQPGRSLRLSCAASGFTFGDKAMNWVRQAPGKGL |
| | PVLVVYDDSDRPSGIPERFSGSNSGNTATLTIERVEAGDEAD | EWVSAISSSGTTMYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 160) |
| E06 | SYELTQPPSVSVAPGKTARITCGGNNIWGKSVHWYQQKPGQA | EVQLVESGGGVVQPGRSLRLSCAASGFTFSSRSMNWVRQAPGKGL |
| | PVLVVYDDIDRPSGIPERFSGSNSGNTATLTISRVEAGDEAD | EWVSAISGSGATKYYADSVKGRFTISRDNSKNTLYLQMNSLRAED |
| | YYCQVWDRGMIFGTGTKLTVL (SEQ ID NO: 110) | TAVYYCARKRWYGMDVWGQGTTVTVSS (SEQ ID NO: 140) |
| E08 | SYELTQPPSVSVAPGKTARITCGGNNIGSKSVHWYQQKPGQA | EVQLVESGGGVVQPGRSLRLSCAASGFTFSRFAMNWVRQAPGKGL |
| | PVLVVYDDSDRPSGIPERFSGSNSGNTATLTISRVEAGDEAD | EWVSAISSSGSTAYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 161) |
| E09 | SYELTQPPSVSVAPGKTARITCGGNNIGSKSVHWYQQKPGQA | EVQLVESGGGVVQPGRSLRLSCAASGFIFSTKAMNWVRQAPGKGL |
| | PVLVVYDDSDRPSGIPERFSGSNSGNTATLTISRVEAGDEAD | EWVSAISSSGATTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 162) |
| E10 | SYELTQPPSVSVAPGKTARITCGGNNIGSKSVHWYQQKPGQA | EVQLVESGGGVVQPGRSLRLSCAASGFTFSNKAMNWVRQAPGKGL |
| | PVLVVYDDSDRPSGIPERFSGSNSGNTATLTISRVEAGDEAD | EWVSAISYEGSTKYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 163) |
| E11 | SYELTQPPSVSVAPGKTARITCGGNNIGSKSVHWYQQKPGQA | EVQLVESGGGVVQPGRSLRLSCAASGFTFSSRSMNWVRQAPGKGL |
| | PVLVVYDDSDRESGIPERFSGSNSGNTATLTISRVEAGDEAD | EWVSAISSSGTTMYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 164) |
| E12 | SYELTQPPSVSVAPGKTARITCGGNNIGSKSVHWYQQKPGQA | EVQLVESGGGVVQPGRSLRLSCAASGFTFGDRAMNWVRQAPGKGL |
| | PVLVVYDDSDRPSGIPERFSGSNSGNTATLTISRVEAGDEAD | EWVSAISSSGATTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 165) |
| F01 | SYELTQPPSVSVAPGKTARITCGGNNIGSKSVHWYQQKPGQA | EVQLVESGGGVVQPGRSLRLSCAASGFTFSSRAMNWVRQAPGKGL |
| | PVLVVYDDSDRPSGIPERFSGSNSGNTATLTISRVEAGDEAD | EWVSAISGSGSTMYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 166) |
| F02 | SYELTQPPSVSVAPGKTARITCGGNNIWGKSVHWYQQKPGQA | EVQLVESGGGVVQPGRSLRLSCAASGFTFSSRSMNWVRQAPGKGD |
| | PVLVVYDDIDRPSGIPERFSGSNSGNTATLTISRVEAGDEAD | EWVSAISGRGDVTYYADSVKGRFTISRDNSKNTLYLQMNSLRAED |
| | YYCQVWDSRTYVFGTGTKLTVL (SEQ ID NO: 106) | TAVYYCARKRWYGMDVWGQGTTVTVSS (SEQ ID NO: 167) |
| F03 | SYELTQPPSVSVAPGKTARITCGGNNIGSKSVHWYQQKPGQA | EVQLVESGGGVVQPGRSLRLSCAASGFTFDNRAMNWVRQAPGKGL |
| | PVLVVYDDSDRPSGIPERFSGSNSGNTATLTISRVEAGDEAD | EWVSAISYEGRTQYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 168) |
| F04 | SYELTQPPSVSVAPGKTARITCGGNNIGSKSVHWYQQKPGQA | EVQLVESGGGVVQPGRSLRLSCAASGFTFSSRSMNWVRQAPGKGL |
| | PVLVVYDDSDRPSGIPERFSGSNSGNTATLTISRVEAGDEAD | EWVSAISGSGDTMYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 169) |
| F06 | SYELTQPPSVSVAPGKTARITCGGNNIGLKSVHWYQQKPGQA | EVQLVESGGGVVQPGRSLRLSCAASGLTFSIYAMNWVRQAPGKGL |
| | PVLVVYDDSDRPSGIPERFSGSNSGNTATLTISRVEAGDEAD | EWVSAISGSGATAYYADSVKGRFTISRDNSKNTLYLQMNSLRAED |
| | YYCQVWDRGMIFGTGTKLTVL (SEQ ID NO: 102) | TAVYYCARKRWYGMDVWGQGTTVTVSS (SEQ ID NO: 170) |
| F07 | SYELTQPPSVSVAPGKTARITCGGNNIGSKSVHWYQQKPGQA | EVQLVESGGGVVQPGRSLRLSCAASGLTFSSRAMNWVRQAPGKGL |
| | PVLVVYDDSDRPSGIPERFSGSNSGNTATLTISRVEAGDEAD | EWVSAISSSGTTTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 171) |
| F08 | SYELTQPPSVSVAPGKTARITCGGNNIGSKSVHWYQQKPGQA | EVQLVESGGGVVQPGRSLRLSCAASGFTFSSRAMNWVRQAPGKGL |
| | PVLVVYDDSDRPSGIPERFSGSNSGNTATLTISRVEAGDEAD | EWVSAISSSGSTAYYADSVKGRFTISRDNSKNTLYLQMNSLRAED |
| | YYCQVWDSRSVVFGTGTKLTVL (SEQ ID NO: 114) | TAVYYCARKRWYGMDVWGQGTTVTVSS (SEQ ID NO: 172) |
| F09 | SYELTQPPSVSVAPGKTARITCGGNNIGSKSVHWYQQKPGQA | EVQLVESGGGVVQPGRSLRLSCAASGFTFSDRAMNWVRQAPGKGL |
| | PVLVVYDDSDRPSGIPERFSGSNSGNTATLTISRVEAGDEAD | EWVSAISGSGKSTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 173) |
| F10 | SYELTQPPSVSVAPGKTARITCGGNNIGSKSVHWYQQKPGQA | EVQLVESGGGVVQPGRSLRLSCAASGFTFSSRAMNWVRQAPGKGL |
| | PVLVVYDDSDRPSGIPERFSGSNSGNTATLTISRVEAGDEAD | EWVSAISGSGATAYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 174) |
| F11 | SYELTQPPSVSVAPGKTARITCGGNNIGSKSVHWYQQKPGQA | EVQLVESGGGVVQPGRSLRLSCAASGFTFSDRAMNWVRQAPGKGL |
| | PVLVVYDDSDRPSGIPERFSGSNSGNTATLTISRVEAGDEAD | EWVSAISGSGFTTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 175) |
| G01 | SYELTQPPSVSVAPGKTARITCGGNNIGMKSVHWYQQKPGQA | EVQLVESGGGVVQPGRSLRLSCAASGFIFSTKAMNWVRQAPGKGL |
| | PVLVVYDDSDRPSGIPERFSGSNSGNTATLTISRVEAGDEAD | EWVSAISGSGATAYYADSVKGRFTISRDNSKNTLYLQMNSLRAED |
| | YYCQVWDRGMIFGTGTKLTVL (SEQ ID NO: 101) | TAVYYCARKRWYGMDVWGQGTTVTVSS (SEQ ID NO: 176) |
| G02 | SYELTQPPSVSVAPGKTARITCGGNNIGMKSVHWYQQKPGQA | EVQLVESGGGVVQPGRSLRLSCAASGFTFDNRAMNWVRQAPGKGL |
| | PVLVVYDDSDRESGIPERFSGSNSGNTATLTISRVEAGDEAD | EWVSAISGSGDTKYYADSVKGRFTISRDNSKNTLYLQMNSLRAED |
| | YYCQVWDRGMIFGTGTKLTVL (SEQ ID NO: 101) | TAVYYCARKRWYGMDVWGQGTTVTVSS (SEQ ID NO: 177) |
| G03 | SYELTQPPSVSVAPGKTARITCQGNNIGYKSVHWYQQKPGQA | EVQLVESGGGVVQPGRSLRLSCAASGFTFTSRAMNWVRQAPGKGL |
| | PVLVVYDDSDRPSGIPERFSGSNSGNTATLTISRVEAGDEAD | EWVSAISASGDTTYYADSVKGRFTISRDNSKNTLYLQMNSLRAED |
| | YYCQVWDRGMIFGTGTKLTVL (SEQ ID NO: 115) | TAVYYCARKRWYGMDVWGQGTTVTVSS (SEQ ID NO: 178) |
| G04 | SYELTQPPSVSVAPGKTARITCGGNNIVTKSVHWYQQKPGQA | EVQLVESGGGVVQPGRSLRLSCAASAFTFSRFAMNWVRQAPGKGL |
| | PVLVVYDDSDRPSGIPERFSGSNSGNTATLTISRVEAGDEAD | EWVSAISSSGSTAYYADSVKGRFTISRDNSKNTLYLQMNSLRAED |
| | YYCQVWDRGMIFGTGTKLTVL (SEQ ID NO: 98) | TAVYYCARKRWYGMDVWGQGTTVTVSS (SEQ ID NO: 179) |
| G05 | SYELTQPPSVSVAPGKTARITCGGNKLGDKWVHWYQQKPGQA | EVQLVESGGGVVQPGRSLRLSCAASGFTFSSRSMNWVRQAPGKGL |
| | PVLVVYEDVDRPSGIPERFSGSNSGNTATLTISRVEAGDEAD | EWVSAISGSGSTVYYADSVKGRFTISRDNSKNTLYLQMNSLRAED |
| | YYCQVWDSRTYVFGTGTKLTVL (SEQ ID NO: 116) | TAVYYCARKRWYGMDVWGQGTTVTVSS (SEQ ID NO: 125) |
| G06 | SYELTQPPSVSVAPGKTARITCGGNNIGLKSVHWYQQKPGQA | EVQLVESGGGVVQPGRSLRLSCAASGFTFDNRAMNWVRQAPGKGL |
| | PVLVVYDDSDRPSGIPERFSGSNSGNTATLTISRVEAGDEAD | EWVSAISSTGKSTYYADSVKGRFTISRDNSKNTLYLQMNSLRAED |
| | YYCQVWDRGMIFGTGTKLTVL (SEQ ID NO: 102) | TAVYYCARKRWYGMDVWGQGTTVTVSS (SEQ ID NO: 180) |
| G07 | SYELTQPPSVSVAPGKTARITCGGNNIGSKSVHWYQQKPGQA | EVQLVESGGGVVQPGRSLRLSCAASGFIFSTKAMNWVRQAPGKGL |
| | PVLVVYDDSDRPSGIPERFSGSNSGNTATLTISRVEAGDEAD | EWVSAISYEGRTQYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 181) |
| G08 | SYELTQPPSVSVAPGKTARITCGGNNIGSKSVHWYQQKPGQA | EVQLVESGGGVVQPGRSLRLSCAASGFTYSSRAMNWVRQAPGKGL |
| | PVLVVYDDSDRPSGIPERFSGSNSGNTATLTISRVEAGDEAD | EWVSAISASGDTTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 182) |
| G09 | SYELTQPPSVSVAPGKTARITCGGNNIGSKSVHWYQQKPGQA | EVQLVESGGGVVQPGRSLRLSCAASGFTFSDRAMNWVRQAPGKGL |
| | PVLVVYDDSDRESGIPERFSGSNSGNTATLTISRVEAGDEAD | EWVSAISASAATTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 183) |
| G10 | SYELTQPPSVSVAPGKTARITCGGNNIGSKSVHWYQQKPGQA | EVQLVESGGGVVQPGRSLRLSCAASGFTFGDRAMNWVRQAPGKGL |
| | PVLVVYDDSDRPSGIPERFSGSNSGNTATLTISRVEAGDEAD | EWVSAISGTGRSTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 184) |
| G11 | SYELTQPPSVSVAPGKTARITCGGNNIGSKSVHWYQQKPGQA | EVQLVESGGGVVQPGRSLRLSCAASGFTFSSRAMNWVRQAPGKGL |
| | PVLVVYDDSDRPSGIPERFSGSNSGNTATLTISRVEAGDEAD | EWVSAISSSGTTMYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 185) |
| G12 | SYELTQPPSVSVAPGKTARITCGGNNIGSKSVHWYQQKPGQA | EVQLVESGGGVVQPGRSLRLSCAASGFTFSSRSMNWVRQAPGKGL |
| | PVLVVYDDSDRPSGIPERFSGSNSGNTATLTISRVEAGDEAD | EWVSAISASGTTIYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 186) |
| H02 | SYELTQPPSVSVAPGKTARITCGGNNIGYKSVHIWYQQKPGQA | EVQLVESGGGVVQPGRSLRLSCAASGFTFSSRAMNWVRQAPGKGL |
| | PVLVVYDDSDRPSGIPERFSGSNSGNTATLTISRVEAGDEAD | EWVSAISASGDTTYYADSVKGRFTISRDNSKNTLYLQMNSLRAED |
| | YYCQVWDSRTYVFGTGTKLTVL (SEQ ID NO: 117) | TAVYYCARKRWYGMDVWGQGTTVTVSS (SEQ ID NO: 145) |
| H03 | SYELTQPPSVSVAPGKTARITCGGNNIGSKSVHWYQQKPGQA | EVQLVESGGGVVQPGRSLRLSCAASGFTFSSRAMNWVRQAPGKGL |
| | PVLVVYDDSDRPSGIPERFSGSNSGNTATLTISRVEAGDEAD | EWVSAISSSGTTTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 187) |
| H05 | SYELTQPPSVSVAPGKTARITCGGNNIGFKSVHWYQQKPGQA | EVQLVESGGGVVQPGRSLRLSCAASGLIFSNFAMNWVRQAPGKGL |
| | PVLVVYDDSDRPSGIPERFSGSNSGNTATLTISRVEAGDEAD | EWVSAISSSGTTMYYADSVKGRFTISRDNSKNTLYLQMNSLRAED |
| | YYCQVWDRGMIFGTGTKLTVL (SEQ ID NO: 118) | TAVYYCARKRWYGMDVWGQGTTVTVSS (SEQ ID NO: 188) |
| H08 | SYELTQPPSVSVAPGKTARITCGGNNIGSKSVHWYQQKPGQA | EVQLVESGGGVVQPGRSLRLSCAASGFTFSSRAMNWVRQAPGKGL |
| | PVLVVYDDSDRPSGIPERFSGSNSGNTATLTISRVEAGDEAD | EWVSAISGPGMTSYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 189) |
| H09 | SYELTQPPSVSVAPGKTARITCGGNNIGSKSVHWYQQKPGQA | EVQLVESGGGVVQPGRSLRLSCAASGFTFSDRAMNWVRQAPGKGL |
| | PVLVVYDDSDRPSGIPERFSGSNSGNTATLTISRVEAGDEAD | EWVSAISGSGTTKYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 190) |
| H11 | SYELTQPPSVSVAPGKTARITCGGNNIGSKSVHWYQQKPGQA | EVQLVESGGGVVQPGRSLRLSCAASGFTFTSRAMNWVRQAPGKGL |
| | PVLVVYDDSDRPSGIPERFSGSNSGNTATLTISRVEAGDEAD | EWVSAISASGDTTYYADSVKGRFTISRDNSKNTLYLQMNSLRAED |
| | YYCQVWDRGMIFGTGTKLTVL (SEQ ID NO: 97) | TAVYYCARKRWYGMDVWGQGTTVTVSS (SEQ ID NO: 178) |

Sequence comparison of the CDRs present in the parental antibody, the maturation libraries, and the identified clones shows that in many cases the amino acid in the parental antibody was optimal since after several selection rounds the clones converge back to the parental sequence. Strikingly, LCDR1 and LCDR2 seemed to tolerate far fewer changes than the other CDRs and for LCDR2, almost all clones reverted to the parental sequence, justifying the inclusion of the parental CDR sequences in the libraries. In LCDR3, despite introducing a diversity of 166,196 different CDRs coming from all) germlines, only 5 different sequences were identified with the most abundant present in 76 out of the 92 clones sequenced. As for HCDR1 and HCDR2, much of the diversity found is concentrated in a few positions, suggesting that these may be less relevant for binding (e.g., position 6 at both HCDR1 at HCDR2), while some other positions showed clear convergence to an amino acid different to the parental.

The number of CDR mutations in the antibodies ranged from only 3 total mutations up to 15 amino acid changes. However, approximately half the clones came from the Combo 2 library in which LCDR1 and LCDR2 were kept constant. That said, only one LCDR1 had 4 mutations from the parental with all others having 2 or fewer. For LCDR3, the dominant sequence had 4 mutations from parental, and for HCDR1 and HCDR2, 3 mutations was the most frequently observed change.

None of the affinity matured antibodies had sequence liabilities in LCDR1, HCDR1, HCDR2, and HCDR3 (this last one remained unchanged from the parental). The GG sequence liability (Kelly, Le et al. 2018) present in the parental HCDR2 was most often replaced by GS, GA, GT, and GD (present in 21, 19, 13, and 11 unique clones respectively). For LCDR3, 67 out of the 81 clones did not have any liabilities, 13 had the same sequence as the parental and therefore retained a DS aspartate isomerization site, and one had the same isomerization site plus a VV polyreactivity motif-probably arising from a synthesis or PCR error since this CDR was not included in the initial design. For LCDR2, only 1 of the 81 unique clones, G05, did not have the DDS aspartate isomerization motif, replaced by an EDV, while 72 had the exact sequence of the parental LCDR2 harboring the liability, 8 others were similar to the parental sequence and still harbored the same motif (again, likely coming from synthesis/PCR errors) (Table 6). In summary, 68 affinity-matured clones had a single CDR liability (Asp isomerization), 12 clones had 2, and 1 clone had 3, showing that the method can be effectively used to reduced developability issues simultaneously with affinity maturation.

TABLE 6
LCDR2 sequences identified in the 81 affinity matured clones. First
sequence correspond to the parental clone and last sequence correspond
to clone G05, not having the Asp isomerization motifs.

| LCDR2 | FREQUENCY |
|---|---|
| DDSDRPS | 72 |
| DDIDRPS | 5 |
| DDSDRSP | 2 |
| DDIDRPA | 1 |
| EDVDRPS | 1 |

Of the identified clones, 24 were expressed as scFv-Fc in yeast: 12 from the Combo 1 library (A01-A02, A03-A06, B01-B06, G05) and 12 from the Combo 2 library (A07-A12. B07-B12). These had 3 to 13 amino acid changes from the parental. To determine whether they retained the same epitope as the initial lead, it was tested whether the clones were able to inhibit the parental scFv from binding to the antigen. The scFv supernatants were incubated with the labeled antigen (10 nM) for 15 minutes, added to the yeast cells displaying the parental molecule to the mixture, and stained to detect binding. All 24 affinity matured clones significantly decreased binding to the antigen by the parental, while a control scFv supernatant directed against an unrelated target did not. While the precise determination of the epitope recognized by all clones would require structural analysis, the fact that all these derive from the parental molecule, retain the same HCDR3 and framework regions, and compete for antigen binding suggests they indeed recognize the same epitope in the target molecule.

Finally, the affinity of the 24 clones was determined using high-throughput surface plasmon resonance (SPR). The supernatants were arrayed by non-covalent capture in a medium-density SPR chip previously coupled with an anti-human polyclonal antibody and (at least 4 replicates per clone). Non-biotinylated antigen was used for all affinity measurements and sensorgrams were fitted using a pseudo-first-order kinetic model (Lundström 1994). The evaluated clones had affinities (K_{D}) on average that were 103 times better than the parental molecule (average 96 pM), dissociation rates (k_{d}) ˜71-334 fold slower, and association rates (kₐ) within the same order of magnitude (Table 7). In fact, it is notable that the off rates for some of the tested antibodies are probably better than 10⁻⁵ (Table 7), as indicated by the bunching of matured antibodies at that value (Table 7)—such slow off rates are challenging to accurately measure using SPR. No affinity difference was observed between clones coming from one combo library versus the other. The improvement of off-rates, but not on-rates, is a reflection of the protocols and selective pressure used during library panning; extended periods of antigen release favored stable binders with long off rates, while no effort was made to rescue clones that had a faster association. Dissociation constants (K_{D}) for the replicates showed good agreement (Table 7).

TABLE 7
Kinetic measurements for affinity matured clones and parental. Reported
values are the mean for at least 4 replicates and standard errors are reported
for the K_{D}. Asterisks indicate clones that reached the minimum value allowed for the
dissociation rate in our measurements (10⁻⁵ s⁻¹).

| Clone | kₐ (M⁻¹ s⁻¹) | k_{d} (s⁻¹) | K_{D} (nM) | K_{D} fold-improvement |
|---|---|---|---|---|
| A01 | 1.7 × 10⁵ | 3.7 × 10⁻⁵ | 0.224 ± 0.02 | 33 |
| A02 | 2.8 × 10⁵ | 1.1 × 10⁻⁵ | 0.039 ± 0.003 | 188 |
| A04 | 3.1 × 10⁵ | 2.1 × 10⁻⁵ | 0.069 ± 0.007 | 108 |
| A05 | 1.9 × 10⁵ | 2.2 × 10⁻⁵ | 0.117 ± 0.01 | 63 |
| A06 | 3.2 × 10⁵ | *1.0 × 10⁻⁵ | 0.034 ± 0.003 | 221 |
| A07 | 1.7 × 10⁵ | 4.7 × 10⁻⁵ | 0.277 ± 0.01 | 27 |
| A08 | 3.1 × 10⁵ | 1.3 × 10⁻⁵ | 0.043 ± 0.007 | 172 |
| A09 | 1.6 × 10⁵ | 1.1 × 10⁻⁵ | 0.074 ± 0.01 | 100 |
| A10 | 1.4 × 10⁵ | 1.4 × 10⁻⁵ | 0.103 ± 0.02 | 72 |
| A11 | 1.9 × 10⁵ | 1.1 × 10⁻⁵ | 0.060 ± 0.007 | 123 |
| A12 | 2.3 × 10⁵ | 3.2 × 10⁻⁵ | 0.139 ± 0.006 | 53 |
| B01 | 3.1 × 10⁵ | *1.0 × 10⁻⁵ | 0.032 ± 0.002 | 231 |
| B02 | 1.8 × 10⁵ | 1.1 × 10⁻⁵ | 0.059 ± 0.004 | 127 |
| B03 | 3.0 × 10⁵ | 2.1 × 10⁻⁵ | 0.070 ± 0.006 | 106 |
| B04 | 1.6 × 10⁵ | 2.7 × 10⁻⁵ | 0.172 ± 0.03 | 43 |
| B05 | 1.5 × 10⁵ | *1.0 × 10⁻⁵ | 0.068 ± 0.0005 | 109 |
| B06 | 1.7 × 10⁵ | 1.7 × 10⁻⁵ | 0.097 ± 0.02 | 76 |
| B07 | 3.3 × 10⁵ | 1.8 × 10⁻⁵ | 0.054 ± 0.006 | 138 |
| B08 | 1.5 × 10⁵ | 1.8 × 10⁻⁵ | 0.117 ± 0.01 | 64 |
| B09 | 2.1 × 10⁵ | 2.5 × 10⁻⁵ | 0.118 ± 0.008 | 63 |
| B10 | 2.6 × 10⁵ | 2.9 × 10⁻⁵ | 0.116 ± 0.009 | 64 |
| B11 | 1.8 × 10⁵ | 1.2 × 10⁻⁵ | 0.068 ± 0.009 | 109 |
| B12 | 3.4 × 10⁵ | 2.8 × 10⁻⁵ | 0.084 ± 0.01 | 89 |
| G05 | 1.6 × 10⁵ | *1.0 × 10⁻⁵ | 0.063 ± 0.003 | 119 |
| Parental | 4.5 × 10⁵ | 3.3 × 10⁻³ | 7.429 ± 0.2 | 1 |

### Materials and Methods

### CDR Design

Two internal naïve human natural phage display libraries (Erasmus, D'Angelo et al. 2021) were sequenced using MiSeq and NovaSeq. Light chain and heavy chain sequences were annotated using IgBlast (Ye, Ma et al. 2013) and the IMGT scheme (Giudicelli, Brochet et al. 2011) was used for CDRs except for LCDR2 where Kabat was used (Kabat, Wu et al. 1979). For LCDR1-2 all IGLV3 family CDRs were considered (family-specific); for LCDR3 all IGLV CDRs (not family-specific); and for HCDR1-2, all 1GHV3 CDRs were considered (family specific). CDRs containing any liabilities (Table 8) were discarded. The remaining CDRs were synthesized as an oligo pool (Agilent Technologies, USA). Flanking framework regions were added to each CDR sequence to enable amplification and assembly.

TABLE 8
List of sequence liabilities removed from CDRs designed for the affinity maturation
libraries. Any CDR containing any of the listed motifs was excluded from the final
oligonucleotide pool. Liabilities are not listed in any particular order of impact.

| Liability | Motifs | Reference |
|---|---|---|
| N-linked glycosylation | N × S and N × T, where × is not a Pro | (Gavel and von Heijne 1990, |
| | | Kumar and Singh 2016) |
| Asn deamidation | NG, NS, NT, NN | (Sydow, Lipsmeier et al. 2014, |
| | | Kumar and Singh 2016) |
| Asp isomerization | DG, DS, DD | (Sydow, Lipsmeier et al. 2014) |
| Polyreactivity | GG, RR, VG, VV, YY, WW, W × W | (Kelly, Le et al. 2018) |
| Aggregation | FHW | (Wu, Luo et al. 2010, Bethea, |
| | | Wu et al. 2012) |
| Unpaired Cys | Odd Cys number | (Kumar and Singh 2016) |
| Proteolysis | DP | (Vlasak and Ionescu 2011) |

### Phase 1 Library Construction

The five different collections of CDR sequences (LCDR1-3, HCDR1-2) were amplified with specific primers by PCR using Q5 polymerase (NEB #M0491L). The remaining regions were amplified from the parental scFv and assembled with the CDRs by PCR. The scFv amplicons from each library were transformed into yeast along with the yeast display vector pSYD previously digested with the enzymes BssHII and NheI (NEB #R0199S and #R0131S) by electroporation using the method described previously (Benatuil, Perez et al. 2010).

### Phase 2 Library Combo Construction

For the Combo 1 library, the regions of interest were amplified from the round 5 populations of each library (L1L2, L3, H1H2) and assembled by PCR For the Combo 2 library, LCDR1 and LCDR2 were amplified from the parental scFv. Assembled scFv libraries were transformed into yeast as before.

### Antigen

The purified monomeric recombinant human protein was ordered from ACRO biosystems and handled according to manufacturer protocols. For use in the yeast display experiments, the protein was chemically biotinylated using EZ-Link NHS-LC-Biotin following the manufacturer's instructions (Thermo Scientific).

### Yeast Display Screening

Yeast display selections were performed as in Ferrara et al. (2012) (Ferrara, Naranjo et al. 2012). Briefly, cells were induced in selective media containing 2% galactose overnight at 20° C. 10⁵ induced cells are washed twice with cold washing buffer (PBS pH 7.4 0.5% BSA) and incubated at room temperature with the biotinylated antigen diluted in PBS. For the equilibrium sort protocol, after the biotinylated antigen incubation step the cells are washed and stained promptly with the anti-SV5 labeled with PE (phycoerythrin; labels cells displaying scFv) and streptavidin labeled with Alexa Fluor 633 (Thermo Scientific; labels cells bound to biotinylated antigen) and then cells binding the antigen are sorted either by FACS (fluorescence-activated cell sorting) or MACS (magnetic-activated cell sorting). For the MACS procedure, paramagnetic beads coated with streptavidin were used (Miltenyi Biotec). For the kinetic sort protocol, after cells are incubated with the biotinylated antigen, they are washed and incubated with unlabeled antigen (10× more concentrated than the biotinylated antigen) to allow for the biotinylated antigen to be released according to the off-rate of the displayed scFv. The unlabeled antigen is added to prevent rebinding of biotinylated antigen. After a defined period of time cells are stained and sorted as described before.

### Screening and Affinity Measurement

The scFv from the final population (round 3, combo libraries) was bulk cloned into a yeast expression vector containing a human IgG1 Fc region to be expressed in the scFv-Fc format. For this, the scFv region was amplified by PCR, digested with BssHII and NheI restriction enzyme (New England Biolabs), and cloned into the pDNL9 vector, 92 clones were analyzed by Sanger sequencing and 24 of these plus the parental were expressed using S. cerevisiae strain YVH10 (ATCC MYA-4940). scFv-Fc fusions were expressed for 72 h at 20° C. in the presence of galactose. The Carterra LSA surface resonance system was used for the affinity measurements. Briefly, anti-Human IgG Fc (Southern Biotech, #2048-01) was covalently coupled to an HC30M chip following the manufacturer's protocols. Crude yeast supernatants containing the scFv-Fc fusions were arrayed on the chip. Five antigen antigens injections were performed, each having a 5-fold increase in concentration (0.16 nM to 100 nM) to determine association and dissociation rates (5 minutes association and 15 minutes dissociation). Data analysis and kinetic parameters calculations were performed using Carterra software.

Binding Inhibition of the Parental scFv by the Affinity Matured Clones

The scFv-Fc supernatants of the same 24 clones used for affinity analysis (plus an unrelated control) were incubated with the labeled antigen (10 nM) for 15 minutes. Approximately 2-105 yeast cells displaying the parental molecule were added to the mixture and incubated for 30 min at room temperature. Cells were washed twice and stained to detect binding using anti-SV5 labeled with PE (phycoerythrin; labels cells displaying scFv) and streptavidin labeled with Alexa Fluor 633. Populations were analyzed by flow cytometry for binding (Intellicyt iQue3 machine). The yeast population displaying the scFv (PE fluorescence) was gated and the median APC fluorescence was used to quantify antigen binding.

### REFERENCES

Akbar, R., P. A. Robert, M. Pavlovic, J. R. Jeliazkov, I. Snapkov, A. Slabodkin, C. R. Weber, L. Scheffer, E. Miho. I. H. Haff, D. T. T. Haug, F. Lund-Johansen, Y. Safonova, G. K. Sandve and V. Greiff (2021). “A compact vocabulary of paratope-epitope interactions enables predictability of antibody-antigen binding.” Cell Rep 34(11): 108856.
Barderas, R., J. Desmet. P. Timmerman, R. Meloen and J. I. Casal (2008). “Affinity maturation of antibodies assisted by in silico modeling.” Proc Natl Acad Sci USA 105(26); 9029-9034.
Benatuil, L., J. M. Perez, J. Belk and C. M. Hsieh (2010). “An improved yeast transformation method for the generation of very large human antibody libraries.” Protein Eng Des Sel 23(4): 155-159.
Bethea, D., S. J. Wu, J. Luo, L. Hyun, E. R. Lacy, A. Teplyakov, S. A. Jacobs, K. T. O'Neil, G. L. Gilliland and Y. Feng (2012). “Mechanisms of self-association of a human monoclonal antibody CNTO607.” Protein Eng Des Sel 25(10): 531-537.
Boder, E. T., K. S. Midelfort and K. D. Wittrup (2000). “Directed evolution of antibody fragments with monovalent femtomolar antigen-binding affinity.” Proc. Natl. Acad Sci. U.S.A. 97(20): 10701-10705.
Boder, E. T. and K. D. Wittrup (1998). “Optimal screening of surface-displayed polypeptide libraries.” Biotechnol Prog 14(1): 55-62. Cannon, D. A., L. Shan, Q. Du, L. Shirinian, K. W. Rickert, K. L. Rosenthal, M.
Korade, 3rd, L. E. van Vlerken-Ysla, A. Buchanan, T. J. Vaughan, M. M. Damschroder and B. Popovic (2019). “Experimentally guided computational antibody affinity maturation with de novo docking, modelling and rational design.” PLoS Comput Biol 15(5): e1006980.
Chan, D. T. Y., L. Jenkinson, S. W. Haynes, M. Austin, A. Diamandakis, D. Burschowsky, C. Seewooruthun, A. Addyman, S. Fiedler, S. Ryman, J. Whitehouse, L. H. Slater, E. Gowans, Y. Shibata, M. Barnard, R. W. Wilkinson, T. J. Vaughan. S. V. Holt, V. Cerundolo, M. D. Carr and M. A. T. Groves (2020). “Extensive sequence and structural evolution of Arginase 2 inhibitory antibodies enabled by an unbiased approach to affinity maturation.” Proc Natl Acad USA 117(29): 16949-16960.
Chou, K.-C., G. Ndmethy, M. Pottle and H. A. Scheraga (1989). “Energy of stabilization of the right-handed p crossover in proteins.” Journal of molecular biology 205(1): 241-249.
Darling, R. J. and P. A. Brault (2004). “Kinetic exclusion assay technology; characterization of molecular interactions.” Assay Drug Dev Technol 2(6): 647-657.
Daugherty, P. S., G. Chen, B. L. Iverson and G. Georgiou (2000). “Quantitative analysis of the effect of the mutation frequency on the affinity maturation of single chain Fv antibodies.” Proc Natl Acad Sci USA 97(5): 2029-2034.
Du, Q., D. Wei and K.-C. Chou (2003). “Correlations of amino acids in proteins.” Peptides 24(12): 1863-1869.
Erasmus, M. F., S. D'Angelo. F. Ferrara, L. Naranjo, A. A. Teixeira, R. Buonpane, S. M. Stewart, H. G. Nastri and A. R. M. Bradbury (2021). “A single donor is sufficient to produce a highly functional in vitro antibody library.” Communications Biology 4(1).
Ferrara, F., L. A. Naranjo, S. Kumar, T. Gaiotto, H. Mukundan, B. Swanson and A. R. Bradbury (2012). “Using phage and yeast display to select hundreds of monoclonal antibodies: application to antigen 85, a tuberculosis biomarker.” PLoS One 7(11): e49535.
Frigotto, L., M. Smith, C. Brankin, A. Sedani, S. Cooper. N. Kanwar, D. Evans, S. Svobodova. C. Baar, J. Glanville, C. Ullman and A. Hine (2015). “Codon-Precise, Synthetic, Antibody Fragment Libraries Built Using Automated Hexamer Codon Additions and Validated through Next Generation Sequencing.” Antibodies 4(2): 88-102.
Garcia-Rodriguez, C., R. Levy, J. W. Armdt, C. M. Forsyth, A. Razai. J. Lou, 1. Geren, R. C. Stevens and J. D. Marks (2007). “Molecular evolution of antibody cross-reactivity for two subtypes of type A botulinum neurotoxin.” Nat Biotechnol 25(1): 107-116.
Gavel, Y. and G. von Heijne (1990). “Sequence differences between glycosylated and non-glycosylated Asn-X-Thr/Ser acceptor sites: implications for protein engineering.” Protein Eng 3(5): 433442.
Giudicelli, V., X. Brochet and M. P. Lefranc (2011). “IMGT/V-QUEST: IMGT standardized analysis of the immunoglobulin (IG) and T cell receptor (TR) nucleotide sequences.” Cold Spring Harb Protoc 2011(6): 695-715.
Gonzalez-Munoz, A., E. Bokma. D. O'Shea. K. Minton, M. Strain. K. Vousden, C. Rossant, L. Jermutus and R Minter (2012). “Tailored amino acid diversity for the evolution of antibody affinity.” MAbs 4(6).
Gram, H., L. A. Marconi, C. F. Barbas, 3rd, T. A. Collet, R. A. Lemer and A. S. Kang (1992). “In vitro selection and affinity maturation of antibodies from a naive combinatorial immunoglobulin library.” Proc Natl Acad Sci USA 89(8): 3576-3580.
Hanes, J., L. Jermutus, S. Weber-Bomhauser, H. R. Bosshard and A. Pluckthun (1998). “Ribosome display efficiently selects and evolves high-affinity antibodies in vitro from immune libraries.” Proc Natl Acad Sci USA 95(24): 14130-14135.
Hanes, J., C. Schaffitzel, A. Knappik and A. Pluckthun (2000). “Picomolar affinity antibodies from a fully synthetic naive library selected and evolved by ribosome display.” Nat. Biotechnol. 18(12): 1287-1292.
Hawkins, R. E., S. J. Russell and G. Winter (1992). “Selection of phage antibodies by binding affinity. Mimicking affinity maturation.” Journal of molecular biology 226(3): 889-896.
Hemminki, A., S. Niemi, L. Hautoniemi, H. Söderlund and K. Takkinen (1998). “Fine tuning of an anti-testosterone antibody binding site by stepwise optimisation of the CDRs.” Immunotechnology 4(1): 59-69.
Hemminki, A., S. Niemi, A. M. Hoffren, L. Hakalahti, H. Soderlund and K. Takkinen (1998). “Specificity improvement of a recombinant anti-testosterone Fab fragment by CDRIII mutagenesis and phage display selection.” Protein Eng 11(4): 311-319.
Hu, D., S. Hu, W. Wan, M. Xu, R. Du, W. Zhao, X. Gao, J. Liu, H. Liu and J. Hong (2015). “Effective Optimization of Antibody Affinity by Phage Display Integrated with High-Throughput DNA Synthesis and Sequencing Technologies.” PLoS One 10(6): e0129125.
Jackson, J. R., G. Sathe, M. Rosenberg and R. Sweet (1995). “In vitro antibody maturation. Improvement of a high affinity, neutralizing antibody against IL-1 beta.” J Immunol 154(7): 3310-3319.
Jain, T., T. Sun, S. Durand, A. Hall, N. R. Houston, J. H. Nett, B. Sharkey, B. Bobrowicz, I. Caffry, Y. Yu, Y. Cao, H. Lynaugh, M. Brown, H. Baruah. L. T. Gray, E. M. Krauland, Y. Xu, M. Vasquez and K. D. Wittrup (2017). “Biophysical properties of the clinical-stage antibody landscape.” Proc Natl Acad Sci USA 114(5): 944-949.
Julian, M. C., C. C. Lee, K. E. Tiller, L. A. Rabia, E. K. Day, A. J. Schick, 3rd and P. M. Tessier (2015). “Co-evolution of affinity and stability of grafted amyloid-motif domain antibodies.” Protein Eng Des Sel 28(10): 339-350.
Julian, M. C., L. Li, S. Garde, R. Wilen and P. M. Tessier (2017). “Efficient affinity maturation of antibody variable domains requires co-selection of compensatory mutations to maintain thermodynamic stability.” Sci Rep 7: 45259.
Kabat, E. A., T. T. Wu and H. Bilofsky (1979). “Evidence supporting somatic assembly of the DNA segments (minigenes), coding for the framework, and complementarity-determining segments of immunoglobulin variable regions.” J Exp Med 149(6): 1299-1313.
Kang, A. S., T. M. Jones and D. R. Burton (1991). “Antibody redesign by chain shuffling from random combinatorial immunoglobulin libraries.” Proc Nad Acad Sci USA 88(24): 11120-11123.
Kelly, R. L., D. Le, J. Zhao and K. D. Wittrup (2018). “Reduction of Nonspecificity Motifs in Synthetic Antibody Libraries.” J Mol Biol 430(1): 119-130.
Kosuri, S. and G. M. Church (2014). “Large-scale de novo DNA synthesis; technologies and applications.” NatMethods 11(5): 499-507.
Kosuri, S., N. Eroshenko, E. M. Leproust, M. Super, J. Way, J. B. Li and G. M. Church (2010). “Scalable gene synthesis by selective amplification of DNA pools from high-fidelity microchips.” Nat Biotechnol 28(12): 1295-1299.
Kovaltsuk, A., J. Leem, S. Kelm, J. Snowden, C. M. Deane and K. Krawczyk (2018). “Observed Antibody Space: A Resource for Data Mining Next-Generation Sequencing of Antibody Repertoires.” J Immunol.
Kumar, S. and S. K. Singh (2016). Developability of Biotherapeutics: computational approaches. Florida, US, CRC Press.
Lamdan, H., J. V. Gavilondo, Y. Munoz, A. Pupo, V. Huerta, A. Musacchio, L. Perez, M. Ayala, G. Rojas, R. F. Balint and J. W. Larrick (2013). “Affinity maturation and fine functional mapping of an antibody fragment against a novel neutralizing epitope on human vascular endothelial growth factor.” Mol Biosyst 9(8): 2097-2106.
Lippow, S. M., K. D. Wittrup and B. Tidor (2007). “Computational design of antibody-affinity improvement beyond in vivo maturation.” Nat Biotechnol 25(10): 1171-1176.
Lou, J., I. Geren, C. Garcia-Rodriguez. C. M. Forsyth, W. Wen, K. Knopp, J. Brown, T. Smith, L. A. Smith and J. D. Marks (2010). “Affinity maturation of human botulinum neurotoxin antibodies by light chain shuffling via yeast mating.” Protein Eng Des Sel 23(4); 311-319.
Low, N. M., P. H. Holliger and G. Winter (1996). “Mimicking somatic hypermutation: affinity maturation of antibodies displayed on bacteriophage using a bacterial mutator strain.” J Mol Biol 260(3): 359-368.
Lu, D., J. Shen, M. D. Vil, H. Zhang, X. Jimenez, P. Bohlen, L. Witte and Z. Zhu (2003). “Tailoring in vitro selection for a picomolar affinity human antibody directed against vascular endothelial growth factor receptor 2 for enhanced neutralizing activity.” The Journal of biological chemistry 278(44): 43496-43507.
Lundström, I. (1994). “Real-time biospecific interaction analysis.” Biosensors and Bioelectronics 9(9-10): 725-736.
Marks, J. D., A. D. Griffiths, M. Malmqvist, T. P. Clackson, J. M. Bye and G. Winter (1992). “By-passing immunization: building high affinity human antibodies by chain shuffling.” Biotechnology (NY) 10(7): 779-783.
Ohlin, M., H. Owman, M. Mach and C. A. K. Borrebaeck (1996). “Light chain shuffling of a high affinity antibody results in a drift in epitope recognition.” Molecular Immunology 33(1): 47-56.
Park, S. G., J. S. Lee, E. Y. Je, I. J. Kim, J. H. Chung and I. H. Choi (2000). “Affinity maturation of natural antibody using a chain shuffling technique and the expression of recombinant antibodies in Escherichia coli.” Biochem Biophys Res Commun 275(2): 553-557.
Pepinsky, R. B., L. Silvian, S. A. Berkowitz, G. Farrington, A. Lugovskoy, L. Walus, J. Eldredge, A. Capili, S. Mi. C. Graff and E. Garber (2010). “Improving the solubility of anti-LINGO-1 monoclonal antibody Li33 by isotype switching and targeted mutagenesis.” Protein Sci 19(5): 954-966.
Rader, C., M. Popkov, J. A. Neves and C. F. Barbas, 3rd (2002). “Integrin alpha(v)beta3 targeted therapy for Kaposi's sarcoma with an in vitro evolved antibody.” FASEB J 16(14): 2000-2002.
Rajpal, A., N. Beyaz, L. Haber, G. Cappuccilli, H. Yee, R. R. Bhatt, T. Takeuchi. R. A. Lemer and R. Crea (2005). “A general method for greatly improving the affinity of antibodies by using combinatorial libraries.” Proc Nad Acad Sci USA 102(24): 8466-8471.
Schier, R., J. Bye, G. Apell. A. McCall. G. P. Adams, M. Malmqvist, L. M. Weiner and J. D. Marks (1996). “Isolation of high-affinity monomeric human anti-c-crbB-2 single chain Fv using affinity-driven selection.” J Mol Biol 255(1): 2843.
Schier, R. and J. D. Marks (1996). “Efficient in vitro affinity maturation of phage antibodies using BIAcore guided selections.” Hum Antibodies Hybridomas 7(3): 97-105.
Schier, R., A. McCall, G. P. Adams, K. W. Marshall, H. Merritt, M. Yim, R. S. Crawford, L. M. Weiner, C. Marks and J. D. Marks (1996). “Isolation of picomolar affinity anti-c-erbB-2 single-chain Fv by molecular evolution of the complementarity determining regions in the center of the antibody binding site.” J Mol Biol 263(4): 551-567.
Sydow, J. F., F. Lipsmeier, V. Larraillet, M. Hilger, B. Mautz, M. Molhoj, J. Kuentzer, S. Klostermann, J. Schoch. H. R. Voelger. J. T. Regula, P. Cramer, A. Papadimitriou and H. Kettenberger (2014). “Structure-based prediction of asparagine and aspartate degradation sites in antibody variable regions.” PLoS One 9(6): e100736.
Thakkar, S., N. Nanaware-Kharade, R. Celikel, E. C. Peterson and K. I. Varughese (2014). “Affinity improvement of a therapeutic antibody to methamphetamine and amphetamine through structure-based antibody engineering.” Sci Rep 4: 3673.
Thompson, J., T. Pope, J. S. Tung, C. Chan, G. Hollis, G. Mark and K. S. Johnson (1996). “Affinity maturation of a high-affinity human monoclonal antibody against the third hypervariable loop of human immunodeficiency virus: use of phage display to improve affinity and broaden strain reactivity.” J Mol Biol 256(1): 77-88.
Tiller, K. E., R. Chowdhury, T. Li, S. D. Ludwig, S. Sen, C. D. Maranas and P. M. Tessier (2017). “Facile Affinity Maturation of Antibody Variable Domains Using Natural Diversity Mutagenesis.” Front Immunol 8: 986.
Tiller, K. E., L. Li, S. Kumar, M. C. Julian, S. Garde and P. M. Tessier (2017). “Arginine mutations in antibody complementarity-determining regions display context-dependent affinity/specificity trade-offs.” J Biol Chem 292(40): 16638-16652.
VanAntwerp, J. J. and K. D. Wittrup (2000). “Fine affinity discrimination by yeast surface display and flow cytometry.” Biotechnol Prog 16(1): 31-37.
Vlasak, J. and R. Ionescu (2011). “Fragmentation of monoclonal antibodies.” MAbs 3(3): 253-263.
Votsmeier, C., H. Plittersdorf, O. Hesse, A. Scheidig. M. Strerath, U. Gritzan, K. Pellengahr, P. Scholz, A. Eicker, D. Myszka, W. M. Coco and U. Haupts (2012). “Femtomolar Fab binding affinities to a protein target by alternative CDR residue co-optimization strategies without phage or cell surface display.” MAbs 4(3): 341-348.
Wu, H., G. Beuerlein, Y. Nie, H. Smith. B. A. Lee, M. Hensler, W. D. Huse and J. D. Watkins (1998). “Stepwise in vitro affinity maturation of Vitaxin, an alphav beta3-specific humanized mAb.” Proc Natl Acad Sci USA 95(11): 6037-6042.
Wu, S. J., J. Luo. K. T. O'Neil, J. Kang, E. R. Lacy, G. Canziani. A. Baker, M. Huang, Q. M. Tang, T. S. Raju, S. A. Jacobs, A. Teplyakov, G. L. Gilliland and Y. Feng (2010). “Structure-based engineering of a monoclonal antibody for improved solubility.” Protein Eng Des Sel 23(8): 643-651.
Xu, J. L. and M. M. Davis (2000). “Diversity in the CDR3 region of V(H) is sufficient for most antibody specificities.” Immunity 13(1): 37-45.
Yang, G. H., S. O. Yoon, M. H. Jang and H. J. Hong (2007). “Affinity maturation of an anti-hepatitis B virus PreS1 humanized antibody by phage display.” J Microbiol 45(6); 528-533.
Yang, W. P., K. Green, S. Pinz-Sweeney, A. T. Briones, D. R. Burton and C. F. Barbas, 3rd (1995). “CDR walking mutagenesis for the affinity maturation of a potent human anti-HIV-1 antibody into the picomolar range.” J Mol Biol 254(3): 392-403.
Ye, J., N. Ma, T. L. Madden and J. M. Ostell (2013). “IgBLAST: an immunoglobulin variable domain sequence analysis tool.” Nucleic Acids Res 41 (Web Server issue): W34-40.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody that binds to human B7-H4, wherein the antibody comprises a light chain variable region (VL) comprising a VL CDR1, a VL CDR2, and a VL CDR3, wherein:
the VL CDR1 comprises the amino acid sequence of any one of SEQ ID NOs:1-12;
the VL CDR2 comprises the amino acid sequence of any one of SEQ ID NOs:13-17;
the VL CDR3 comprises the amino acid sequence of any one of SEQ ID NOs:18-22;
the VH CDR1 comprises the amino acid sequence of any one of SEQ ID NOs:23-43;
the VH CDR2 comprises the amino acid sequence of any one of SEQ ID NOs:44-89;
and the VH CDR3 comprises the amino acid sequence of SEQ ID NO:90.

2. The antibody of claim 1, wherein the VL CDR1, the VL CDR2, and the VL CDR3 each correspond to the VL CDRs set forth in Table 4 for a single VL clone, and wherein the VH CDR1, the VH CDR2, and the VH CDR3 each correspond to the VH CDRs set forth in Table 4 for a single VH clone.

3. The antibody of claim 1, wherein;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:23; the VH CDR2 comprises the amino acid sequence of SEQ ID NO:44; and the VH CDR3 comprises the amino acid sequence of SEQ ID NO:90;
the VL CDR1 comprises the amino acid sequence of SEQ ID NO.1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:24; the VH CDR2 comprises the amino acid sequence of SEQ ID NO:45; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:25; the VH CDR2 comprises the amino acid sequence of SEQ ID NO:46; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:47; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:27; the VH CDR2 comprises the amino acid sequence of SEQ ID NO:48; and the VH CDR3 comprises the amino acid sequence of SEQ ID NO:90;
the VL CDR1 comprises the amino acid sequence of SEQ ID NO:2; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:28; the VH CDR2 comprises the amino acid sequence of SEQ ID NO:49; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:50; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:28; the VH CDR2 comprises the amino acid sequence of SEQ ID NO:51; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:52; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:53; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:50; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:29; the VH CDR2 comprises the amino acid sequence of SEQ ID NO:54; and the VH CDR3 comprises the amino acid sequence of SEQ ID NO:90;
the VL CDR1 comprises the amino acid sequence of SEQ ID NO:3; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:30; the VH CDR2 comprises the amino acid sequence of SEQ ID NO:46; and the VH CDR3 comprises the amino acid sequence of SEQ ID NO:90;
the VL CDR1 comprises the amino acid sequence of SEQ ID NO:3; the VL CDR2 comprises the amino acid sequence of SEQ ID NO:14; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:50; and the VH CDR3 comprises the amino acid sequence of SEQ ID NO:90;
the VL CDR1 comprises the amino acid sequence of SEQ ID NO:4; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:28; the VH CDR2 comprises the amino acid sequence of SEQ ID NO:55; and the VH CDR3 comprises the amino acid sequence of SEQ ID NO:90;
the VL CDR1 comprises the amino acid sequence of SEQ ID NO:5; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:31; the VH CDR2 comprises the amino acid sequence of SEQ ID NO:56; and the VH CDR3 comprises the amino acid sequence of SEQ ID NO:90;
the VL CDR1 comprises the amino acid sequence of SEQ ID NO:6; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:32; the VH CDR2 comprises the amino acid sequence of SEQ ID NO:49; and the VH CDR3 comprises the amino acid sequence of SEQ ID NO:90;
the VL CDR1 comprises the amino acid sequence of SEQ ID NO:7; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:24; the VH CDR2 comprises the amino acid sequence of SEQ ID NO:45; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:28; the VH CDR2 comprises the amino acid sequence of SEQ ID NO:45; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:57; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:27; the VH CDR2 comprises the amino acid sequence of SEQ ID NO:58; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:20; the VH CDR1 comprises the amino acid sequence of SEQ ID NO:33; the VH CDR2 comprises the amino acid sequence of SEQ ID NO:59; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:25; the VH CDR2 comprises the amino acid sequence of SEQ ID NO:60; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:61; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:29; the VH CDR2 comprises the amino acid sequence of SEQ ID NO:54; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:62; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:63; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:28; the VH CDR2 comprises the amino acid sequence of SEQ ID NO:64; and the VH CDR3 comprises the amino acid sequence of SEQ ID NO:90;
the VL CDR1 comprises the amino acid sequence of SEQ ID NO:8; the VL CDR2 comprises the amino acid sequence of SEQ ID NO:15; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:50; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:65; and the VH CDR3 comprises the amino acid sequence of SEQ ID NO:90;
the VL CDR1 comprises the amino acid sequence of SEQ ID NO:9; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:31; the VH CDR2 comprises the amino acid sequence of SEQ ID NO:66; and the VH CDR3 comprises the amino acid sequence of SEQ ID NO:90;
the VL CDR1 comprises the amino acid sequence of SEQ ID NO:3, the VL CDR2 comprises the amino acid sequence of SEQ ID NO:14; the VL CDR3 comprises the amino acid sequence of SEQ ID NO:19; the VH CDR1 comprises the amino acid sequence of SEQ ID NO:35; the VH CDR2 comprises the amino acid sequence of SEQ ID NO:46; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:36; the VH CDR2 comprises the amino acid sequence of SEQ ID NO:51; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:67; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:51; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:33; the VH CDR2 comprises the amino acid sequence of SEQ ID NO:68; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:21; the VH CDR1 comprises the amino acid sequence of SEQ ID NO:27; the VH CDR2 comprises the amino acid sequence of SEQ ID NO:65; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:37; the VH CDR2 comprises the amino acid sequence of SEQ ID NO:60; and the VH CDR3 comprises the amino acid sequence of SEQ ID NO:90;
the VL CDR1 comprises the amino acid sequence of SEQ ID NO:4; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:69; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:16; the VL CDR3 comprises the amino acid sequence of SEQ ID NO:19; the VH CDR1 comprises the amino acid sequence of SEQ ID NO:31; the VH CDR2 comprises the amino acid sequence of SEQ ID NO:70; and the VH CDR3 comprises the amino acid sequence of SEQ ID NO:90;
the VL CDR1 comprises the amino acid sequence of SEQ ID NO:8; the VL CDR2 comprises the amino acid sequence of SEQ ID NO:15; the VL CDR3 comprises the amino acid sequence of SEQ ID NO:19; the VH CDR1 comprises the amino acid sequence of SEQ ID NO:33; the VH CDR2 comprises the amino acid sequence of SEQ ID NO:59; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:64; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:27; the VH CDR2 comprises the amino acid sequence of SEQ ID NO:71; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:33; the VH CDR2 comprises the amino acid sequence of SEQ ID NO:48; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:72; and the VH CDR3 comprises the amino acid sequence of SEQ ID NO:90;
the VL CDR1 comprises the amino acid sequence of SEQ ID NO:2; the VL CDR2 comprises the amino acid sequence of SEQ ID NO:15; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:73; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:24; the VH CDR2 comprises the amino acid sequence of SEQ ID NO:74; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:75; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:28; the VH CDR2 comprises the amino acid sequence of SEQ ID NO:66; and the VH CDR3 comprises the amino acid sequence of SEQ ID NO:90;
the VL CDR1 comprises the amino acid sequence of SEQ ID NO:9; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:76; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:38; the VH CDR2 comprises the amino acid sequence of SEQ ID NO:75; and the VH CDR3 comprises the amino acid sequence of SEQ ID NO:90;
the VL CDR1 comprises the amino acid sequence of SEQ ID NO:8; the VL CDR2 comprises the amino acid sequence of SEQ ID NO:15; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:65; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:39; the VH CDR2 comprises the amino acid sequence of SEQ ID NO:76; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:40; the VH CDR2 comprises the amino acid sequence of SEQ ID NO:66; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:77; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:60; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:78; and the VH CDR3 comprises the amino acid sequence of SEQ ID NO:90;
the VL CDR1 comprises the amino acid sequence of SEQ ID NO:8; the VL CDR2 comprises the amino acid sequence of SEQ ID NO:15; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:79; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:29; the VH CDR2 comprises the amino acid sequence of SEQ ID NO:46; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:80; and the VH CDR3 comprises the amino acid sequence of SEQ ID NO:90;
the VL CDR1 comprises the amino acid sequence of SEQ ID NO:5; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:41; the VH CDR2 comprises the amino acid sequence of SEQ ID NO:60; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:32; the VH CDR2 comprises the amino acid sequence of SEQ ID NO:58; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:22; the VH CDR1 comprises the amino acid sequence of SEQ ID NO:34; the VH CDR2 comprises the amino acid sequence of SEQ ID NO:76; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:81; and the VH CDR3 comprises the amino acid sequence of SEQ ID NO:90;
the VL CDR1 comprises the amino acid sequence of SEQ ID NO:4; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:40; the VH CDR2 comprises the amino acid sequence of SEQ ID NO:60; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:28; the VH CDR2 comprises the amino acid sequence of SEQ ID NO:82; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:75; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:26; the VH CDR2 comprises the amino acid sequence of SEQ ID NO:83; and the VH CDR3 comprises the amino acid sequence of SEQ ID NO:90;
the VL CDR1 comprises the amino acid sequence of SEQ ID NO:4; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:29; the VH CDR2 comprises the amino acid sequence of SEQ ID NO:84; and the VH CDR3 comprises the amino acid sequence of SEQ ID NO:90;
the VL CDR1 comprises the amino acid sequence of SEQ ID NO:10; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:25; the VH CDR2 comprises the amino acid sequence of SEQ ID NO:62; and the VH CDR3 comprises the amino acid sequence of SEQ ID NO:90;
the VL CDR1 comprises the amino acid sequence of SEQ ID NO:2; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:42; the VH CDR2 comprises the amino acid sequence of SEQ ID NO:76; and the VH CDR3 comprises the amino acid sequence of SEQ ID NO:90;
the VL CDR1 comprises the amino acid sequence of SEQ ID NO:11; the VL CDR2 comprises the amino acid sequence of SEQ ID NO:17; the VL CDR3 comprises the amino acid sequence of SEQ ID NO:18; the VH CDR1 comprises the amino acid sequence of SEQ ID NO:26; the VH CDR2 comprises the amino acid sequence of SEQ ID NO:50; and the VH CDR3 comprises the amino acid sequence of SEQ ID NO:90;
the VL CDR1 comprises the amino acid sequence of SEQ ID NO:5; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:29; the VH CDR2 comprises the amino acid sequence of SEQ ID NO:85; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:40; the VH CDR2 comprises the amino acid sequence of SEQ ID NO:46; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:27; the VH CDR2 comprises the amino acid sequence of SEQ ID NO:62; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:86; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:25; the VH CDR2 comprises the amino acid sequence of SEQ ID NO:62; and the VH CDR3 comprises the amino acid sequence of SEQ ID NO:90;
the VL CDR1 comprises the amino acid sequence of SEQ ID NO:10; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:62; and the VH CDR3 comprises the amino acid sequence of SEQ ID NO:90;
the VL CDR1 comprises the amino acid sequence of SEQ ID NO: 1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:58; and the VH CDR3 comprises the amino acid sequence of SEQ ID NO:90;
the VL CDR1 comprises the amino acid sequence of SEQ ID NO:12; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:43; the VH CDR2 comprises the amino acid sequence of SEQ ID NO:75; and the VH CDR3 comprises the amino acid sequence of SEQ ID NO:90;
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:34; the VH CDR2 comprises the amino acid sequence of SEQ ID NO:87; and the VH CDR3 comprises the amino acid sequence of SEQ ID NO:90; or
the VL CDR1 comprises the amino acid sequence of SEQ ID NO:1; the VL CDR2 comprises the amino acid sequence of SEQ ID NO:13; the VL CDR3 comprises the amino acid sequence of SEQ ID NO:19; the VH CDR1 comprises the amino acid sequence of SEQ ID NO:30; the VH CDR2 comprises the amino acid sequence of SEQ ID NO:88; and the VH CDR3 comprises the amino acid sequence of SEQ ID NO:90.

4. The antibody of claim 1, wherein
the VL comprises the amino acid sequence of any one of SEQ ID NOs:96-118; and
the VH comprises the amino acid sequence of any one of SEQ ID NOs:119-190.

5. The antibody of claim 1, wherein;
the VL comprises the amino acid sequence of SEQ ID NO:96 and the VH comprises the amino acid sequence of SEQ ID NO:119;
the VL comprises the amino acid sequence of SEQ ID NO:97 and the VH comprises the amino acid sequence of SEQ ID NO:120;
the VL comprises the amino acid sequence of SEQ ID NO:97 and the VH comprises the amino acid sequence of SEQ ID NO:121;
the VL comprises the amino acid sequence of SEQ ID NO:97 and the VH comprises the amino acid sequence of SEQ ID NO:122;
the VL comprises the amino acid sequence of SEQ ID NO:97 and the VH comprises the amino acid sequence of SEQ ID NO:123;
the VL comprises the amino acid sequence of SEQ ID NO:98 and the VH comprises the amino acid sequence of SEQ ID NO:124;
the VL comprises the amino acid sequence of SEQ ID NO:96 and the VH comprises the amino acid sequence of SEQ ID NO:125;
the VL comprises the amino acid sequence of SEQ ID NO:97 and the VH comprises the amino acid sequence of SEQ ID NO:126;
the VL comprises the amino acid sequence of SEQ ID NO:97 and the VH comprises the amino acid sequence of SEQ ID NO:127;
the VL comprises the amino acid sequence of SEQ ID NO:97 and the VH comprises the amino acid sequence of SEQ ID NO:128;
the VL comprises the amino acid sequence of SEQ ID NO:97 and the VH comprises the amino acid sequence of SEQ ID NO:125;
the VL comprises the amino acid sequence of SEQ ID NO:96 and the VH comprises the amino acid sequence of SEQ ID NO:129;
the VL comprises the amino acid sequence of SEQ ID NO:99 and the VH comprises the amino acid sequence of SEQ ID NO:130;
the VL comprises the amino acid sequence of SEQ ID NO:100 and the VH comprises the amino acid sequence of SEQ ID NO:125;
the VL comprises the amino acid sequence of SEQ ID NO:101 and the VH comprises the amino acid sequence of SEQ ID NO:131;
the VL comprises the amino acid sequence of SEQ ID NO:102 and the VH comprises the amino acid sequence of SEQ ID NO:132;
the VL comprises the amino acid sequence of SEQ ID NO: 103 and the VH comprises the amino acid sequence of SEQ ID NO:133;
the VL comprises the amino acid sequence of SEQ ID NO:104 and the VH comprises the amino acid sequence of SEQ ID NO:120;
the VL comprises the amino acid sequence of SEQ ID NO:97 and the VH comprises the amino acid sequence of SEQ ID NO:134;
the VL comprises the amino acid sequence of SEQ ID NO:97 and the VH comprises the amino acid sequence of SEQ ID NO:135;
the VL comprises the amino acid sequence of SEQ ID NO:97 and the VH comprises the amino acid sequence of SEQ ID NO:136;
the VL comprises the amino acid sequence of SEQ ID NO:105 and the VH comprises the amino acid sequence of SEQ ID NO:137;
the VL comprises the amino acid sequence of SEQ ID NO:97 and the VH comprises the amino acid sequence of SEQ ID NO:138;
the VL comprises the amino acid sequence of SEQ ID NO:97 and the VH comprises the amino acid sequence of SEQ ID NO:139;
the VL comprises the amino acid sequence of SEQ ID NO:97 and the VH comprises the amino acid sequence of SEQ ID NO:129;
the VL comprises the amino acid sequence of SEQ ID NO:106 and the VH comprises the amino acid sequence of SEQ ID NO:125;
the VL comprises the amino acid sequence of SEQ ID NO:96 and the VH comprises the amino acid sequence of SEQ ID NO:140;
the VL comprises the amino acid sequence of SEQ ID NO:107 and the VH comprises the amino acid sequence of SEQ ID NO:141;
the VL comprises the amino acid sequence of SEQ ID NO:108 and the VH comprises the amino acid sequence of SEQ ID NO:142;
the VL comprises the amino acid sequence of SEQ ID NO:97 and the VH comprises the amino acid sequence of SEQ ID NO:143;
the VL comprises the amino acid sequence of SEQ ID NO:97 and the VH comprises the amino acid sequence of SEQ ID NO:144;
the VL comprises the amino acid sequence of SEQ ID NO:97 and the VH comprises the amino acid sequence of SEQ ID NO:145;
the VL comprises the amino acid sequence of SEQ ID NO:97 and the VH comprises the amino acid sequence of SEQ ID NO:146;
the VL comprises the amino acid sequence of SEQ ID NO:97 and the VH comprises the amino acid sequence of SEQ ID NO:147;
the VL comprises the amino acid sequence of SEQ ID NO:97 and the VH comprises the amino acid sequence of SEQ ID NO:148;
the VL comprises the amino acid sequence of SEQ ID NO:96 and the VH comprises the amino acid sequence of SEQ ID NO:149;
the VL comprises the amino acid sequence of SEQ ID NO:101 and the VH comprises the amino acid sequence of SEQ ID NO:150;
the VL comprises the amino acid sequence of SEQ ID NO:109 and the VH comprises the amino acid sequence of SEQ ID NO:151;
the VL comprises the amino acid sequence of SEQ ID NO:110 and the VH comprises the amino acid sequence of SEQ ID NO:137;
the VL comprises the amino acid sequence of SEQ ID NO:96 and the VH comprises the amino acid sequence of SEQ ID NO:152;
the VL comprises the amino acid sequence of SEQ ID NO:97 and the VH comprises the amino acid sequence of SEQ ID NO:153;
the VL comprises the amino acid sequence of SEQ ID NO:97 and the VH comprises the amino acid sequence of SEQ ID NO:154;
the VL comprises the amino acid sequence of SEQ ID NO:97 and the VH comprises the amino acid sequence of SEQ ID NO:155;
the VL comprises the amino acid sequence of SEQ ID NO:97 and the VH comprises the amino acid sequence of SEQ ID NO:156;
the VL comprises the amino acid sequence of SEQ ID NO:111 and the VH comprises the amino acid sequence of SEQ ID NO:157;
the VL comprises the amino acid sequence of SEQ ID NO:112 and the VH comprises the amino acid sequence of SEQ ID NO:158;
the VL comprises the amino acid sequence of SEQ ID NO: 113 and the VH comprises the amino acid sequence of SEQ ID NO:159;
the VL comprises the amino acid sequence of SEQ ID NO:97 and the VH comprises the amino acid sequence of SEQ ID NO:160;
the VL comprises the amino acid sequence of SEQ ID NO:110 and the VH comprises the amino acid sequence of SEQ ID NO:140;
the VL comprises the amino acid sequence of SEQ ID NO:97 and the VH comprises the amino acid sequence of SEQ ID NO:161;
the VL comprises the amino acid sequence of SEQ ID NO:97 and the VH comprises the amino acid sequence of SEQ ID NO:162;
the VL comprises the amino acid sequence of SEQ ID NO:97 and the VH comprises the amino acid sequence of SEQ ID NO:163;
the VL comprises the amino acid sequence of SEQ ID NO:97 and the VH comprises the amino acid sequence of SEQ ID NO:164;
the VL comprises the amino acid sequence of SEQ ID NO:97 and the VH comprises the amino acid sequence of SEQ ID NO:165;
the VL comprises the amino acid sequence of SEQ ID NO:97 and the VH comprises the amino acid sequence of SEQ ID NO:166;
the VL comprises the amino acid sequence of SEQ ID NO:106 and the VH comprises the amino acid sequence of SEQ ID NO:167;
the VL comprises the amino acid sequence of SEQ ID NO:97 and the VH comprises the amino acid sequence of SEQ ID NO:168;
the VL comprises the amino acid sequence of SEQ ID NO:97 and the VH comprises the amino acid sequence of SEQ ID NO:169;
the VL comprises the amino acid sequence of SEQ ID NO:102 and the VH comprises the amino acid sequence of SEQ ID NO:170;
the VL comprises the amino acid sequence of SEQ ID NO:97 and the VH comprises the amino acid sequence of SEQ ID NO:171;
the VL comprises the amino acid sequence of SEQ ID NO:114 and the VH comprises the amino acid sequence of SEQ ID NO:172;
the VL comprises the amino acid sequence of SEQ ID NO:97 and the VH comprises the amino acid sequence of SEQ ID NO:173;
the VL comprises the amino acid sequence of SEQ ID NO:97 and the VH comprises the amino acid sequence of SEQ ID NO:174;
the VL comprises the amino acid sequence of SEQ ID NO:97 and the VH comprises the amino acid sequence of SEQ ID NO:175;
the VL comprises the amino acid sequence of SEQ ID NO:101 and the VH comprises the amino acid sequence of SEQ ID NO:176;
the VL comprises the amino acid sequence of SEQ ID NO:101 and the VH comprises the amino acid sequence of SEQ ID NO:177;
the VL comprises the amino acid sequence of SEQ ID NO:115 and the VH comprises the amino acid sequence of SEQ ID NO:178;
the VL comprises the amino acid sequence of SEQ ID NO:98 and the VH comprises the amino acid sequence of SEQ ID NO:179;
the VL comprises the amino acid sequence of SEQ ID NO:116 and the VH comprises the amino acid sequence of SEQ ID NO:125;
the VL comprises the amino acid sequence of SEQ ID NO:102 and the VH comprises the amino acid sequence of SEQ ID NO:180;
the VL comprises the amino acid sequence of SEQ ID NO:97 and the VH comprises the amino acid sequence of SEQ ID NO:181;
the VL comprises the amino acid sequence of SEQ ID NO:97 and the VH comprises the amino acid sequence of SEQ ID NO:182;
the VL comprises the amino acid sequence of SEQ ID NO:97 and the VH comprises the amino acid sequence of SEQ ID NO:183;
the VL comprises the amino acid sequence of SEQ ID NO:97 and the VH comprises the amino acid sequence of SEQ ID NO:184;
the VL comprises the amino acid sequence of SEQ ID NO:97 and the VH comprises the amino acid sequence of SEQ ID NO:185;
the VL comprises the amino acid sequence of SEQ ID NO:97 and the VH comprises the amino acid sequence of SEQ ID NO:186;
the VL comprises the amino acid sequence of SEQ ID NO: 117 and the VH comprises the amino acid sequence of SEQ ID NO:145;
the VL comprises the amino acid sequence of SEQ ID NO:97 and the VH comprises the amino acid sequence of SEQ ID NO:187;
the VL comprises the amino acid sequence of SEQ ID NO:118 and the VH comprises the amino acid sequence of SEQ ID NO:188;
the VL comprises the amino acid sequence of SEQ ID NO:97 and the VH comprises the amino acid sequence of SEQ ID NO:189;
the VL comprises the amino acid sequence of SEQ ID NO:97 and the VH comprises the amino acid sequence of SEQ ID NO:190; or
the VL comprises the amino acid sequence of SEQ ID NO:97 and the VH comprises the amino acid sequence of SEQ ID NO:178.

6. The antibody of any one of claims 1-5, wherein the antibody is a full-length antibody.

7. The antibody of any one of claims 1-6, wherein the antibody is a bispecific antibody, a single chain antibody (scFv), an Fab fragment, an F(ab′)2 fragment, an Fab′ fragment, an Fv fragment, an sc(Fv)₂, or a diabody.

8. The antibody of any one of claims 1-7, which is conjugated to a toxic substance.

9. The antibody of claim 8, wherein the toxic substance is a radioisotope or a cytotoxic agent.

10. The antibody of any one of claims 1-9, which is conjugated to a detectable label.

11. A nucleic acid or a set of nucleic acids, which collectively encodes the antibody of any one of claims 1-10.

12. An expression vector or a set of expression vectors comprising the nucleic acid or the set of nucleic acids of claim 11 operably linked to a promoter.

13. An isolated cell comprising the nucleic acid or the set of nucleic acids of claim 11 or the expression vector or the set of expression vectors of claim 12.

14. A method of making the antibody of any one of claims 1-10, comprising culturing the cell of claim 13 and isolating the antibody.

15. A pharmaceutical composition comprising the antibody of any one of claims 1-10, the nucleic acid or the set of nucleic acids of claim 11, the expression vector or the set of expression vectors of claim 12, or the isolated cell of claim 13, and a pharmaceutically acceptable carrier.

16. A method of treating a cancer in a human subject in need thereof, the method comprising administering to the human subject an effective amount of the antibody of any one of claims 1-10 or the pharmaceutical composition of claim 15.

17. The method of claim 16, wherein the cancer is ovarian cancer, breast cancer, endometrial cancer, bladder cancer, esophageal cancer, oral squamous cell carcinoma, brain cancer, prostate cancer, pancreatic cancer, cervical cancer, skin cancer, lung cancer, gastric cancer, or renal cell carcinoma.

18. The method of claim 16 or claim 17, further comprising administering to the human subject an additional therapy selected from the group consisting of a Janus tyrosine kinase (JAK) inhibitor, a phosphoinositide 3-kinase (PI3K) inhibitor, a standard of care therapy, or a combination thereof.

19. The method of claim 18, wherein the JAK inhibitor is ruxolitinib and itaticinib.

20. The method of claim 18, wherein the PI3K inhibitor is parsaclisib.

21. The method of claim 18, wherein the standard of care therapy is selected from the group consisting of a chemotherapy, a radiation therapy, a surgical therapy, an immunotherapy, or a combination thereof.

22. A method for detecting human B7-H4, the method comprising:
contacting the antibody of any one of claims 1-10 with a sample suspected of containing B7-H4, and
detecting binding of the antibody to B7-H4.

23. The method of claim 22, wherein B7-H4 is expressed on a cell surface.

24. The method of claim 22 or claim 23, wherein the contacting step is performed by administering the antibody to a subject.

25. A kit comprising the antibody of any one of claims 1-10, the nucleic acid or the set of nucleic acids of claim 11, the expression vector or the set of expression vectors of claim 12, or the isolated cell of claim 13, and instructions for use in treating a cancer in a human subject in need thereof, optionally with instructions for use in combination with an additional therapy.
